(19) United States
(12) Patent Application Publication
(10) Pub. No.: US 2019/0021998 A1
(43) Pub. Date: Jan. 24, 2019

(54) TOPICAL NANOEMULSION THERAPY FOR WOUNDS

(71) Applicant: THE REGENTS OF THE UNIVERSITY OF MICHIGAN, Ann Arbor, MI (US)

(72) Inventors: Mark R. HEMMILA, Ann Arbor, MI (US); James J. BAKER, JR., Ann Arbor, MI (US); Vladislav A. DOLGACHEV, Ann Arbor, MI (US); Susan M. CIOTTI, Ann Arbor, MI (US); Suhe WANG, Ann Arbor, MI (US)

(21) Appl. No.: 16/049,371

(22) Filed: Jul. 30, 2018

Related U.S. Application Data
(63) Continuation of application No. 15/127,967, filed on Sep. 21, 2016, now abandoned.
(60) Provisional application No. 61/968,868, filed on Mar. 21, 2014.

(51) Int. Cl. A61K 9/107; A61K 47/34; A61K 47/26; A61K 47/18; A61K 47/10; A61K 9/00; A61K 47/44; A61K 31/14

(52) U.S. Cl. CPC A61K 9/1075; A61K 47/34; A61K 47/26; A61K 47/183; A61K 47/10; A61K 31/14; A61K 47/44; A61K 47/186; A61K 9/0014

(57) ABSTRACT

The present invention relates to therapeutic nanoemulsion compositions and to methods of utilizing the same to treat a burn wound. In particular, nanoemulsion compositions are described herein that find use in reducing and/or preventing progression/conversion of a partial thickness burn wound (e.g., to deep partial thickness wound or a full thickness burn wound (e.g., by accelerating and/or improving burn wound healing)). Compositions and methods of the present invention find use in, among other things, clinical (e.g. therapeutic and preventative medicine), industrial, and research applications.

### CROSS-REFERENCE TO RELATED APPLICATIONS

This application is a continuation of U.S. patent application Ser. No. 15/172,967, filed Sep. 21, 2016, which is a § 371 U.S. National Entry Application of International. Patent Application No. PCT/US2015/021854, filed Mar. 20, 2015, which claims the benefit of U.S. Provisional Application No. 61/968,868, filed Mar. 21, 2014, the disclosure of each of which is incorporated herein by reference in its entirety.

### STATEMENT REGARDING FEDERALLY SPONSORED RESEARCH OR DEVELOPMENT

This invention was made with government support under W81XWH-11-2-0005 awarded by the US Army Medical Research Materiel Command. The Government has certain rights in the invention.

### FIELD OF THE INVENTION

The present invention relates to therapeutic nanoemulsion compositions and to methods of utilizing the same to treat a wound (e.g., a burn wound). In particular, nanoemulsion compositions are described herein that find use in reducing and/or preventing progression/conversion of a partial thickness burn wound (e.g., to deep partial thickness wound or a full thickness burn wound (e.g., by accelerating and/or improving burn wound healing)). Compositions and methods of the present invention find use in, among other things, clinical (e.g. therapeutic and preventative medicine), industrial, and research applications.

### BACKGROUND OF THE INVENTION

Contemporary burn wound management involves early debridement and reconstruction of non-viable skin coupled with provision of supportive care and topical antimicrobial dressing changes to partial thickness burn wounds. The goal of modern burn wound care is to provide an optimal environment for epidermal renewal. Restoration of skin integrity takes place via regrowth of keratinocytes from preserved hair follicles or transfer of split thickness skin grafts harvested from non-burn regions. During the period of epidermal renewal it is important to avoid further injury to the skin, abrogate burn wound progression, and minimize secondary complications such as wound infection.

Early excision of full-thickness burn eschar, immediate skin grafting, and treatment of remaining open or partial thickness areas of burn wound with topical antimicrobial agents has heretofore been the most effective way of minimizing burn wound colonization and invasive wound infection. (See, e.g., Bessey, Wound care. In Herndon DN, ed: Total Burn Care 3^{rd} edition. Philadelphia, Pa.: Elsevier Inc., 2007, pp 127-135.). Popular topical antimicrobial agents include silver sulfadiazine (SILVADENE), mafenide acetate (SULFAMYLON), and colloidal silver impregnated dressings (ACTICOAT, SILVERLON). Each of these agents has potential limitations such as variable ability to penetrate eschar, uneven efficacy against both Gram-negative and Gram-positive bacteria, and potential toxicity to host immune cells (See, e.g., Steinstraesser et al., Antimicrob Agents Chemother 46(6):1837-1844, 2002).

### SUMMARY OF THE INVENTION

The present invention relates to therapeutic nanoemulsion compositions and to methods of utilizing the same to treat a burn wound. In particular, nanoemulsion compositions are described herein that find use in reducing and/or preventing progression/conversion of a partial thickness burn wound (e.g., to deep partial thickness wound or a full thickness burn wound (e.g., by accelerating and/or improving burn wound healing)). Compositions and methods of the present invention find use in, among other things, clinical (e.g. therapeutic and preventative medicine), industrial, and research applications.

Accordingly, in some embodiments, the invention provides compositions and methods for treating burn wounds. For example, in some embodiments, the present invention provides a method of treating a burn wound comprising providing a subject harboring a burn wound; and a composition comprising a nanoemulsion described herein; and administering the composition comprising a nanoemulsion to the burn wound, wherein the administering treats the burn wound (e.g., prevents the progression and/or convervsion of a partial thickness burn wound to a deep partial thickness burn wound or to a full thickness burn wound). A variety of nanoemulsions that find use in the methods of the invention are described herein. The invention is not limited by the amount of nanoemulsion utilized, the frequency of administration and/or the duration of administration. Indeed, therapeutically effective amounts of a nanoemulsion are described herein. In some embodiments, administration of a nanoemulsion to a burn wound inhibits the expression of IL-1β at the burn wound site. In further embodiments, administration of the nanoemulsion inhibits bacterial growth at the burn wound site. In preferred embodiments, administration of the nanoemulsion inhibits ischemic necrosis. In other preferred embodiments, administration of the nanoemulsion inhibits protein denaturation.

The invention also provides a method of increasing skin regeneration within a burn wound (e.g., a superficial burn wound, a partial thickness burn wound, a deep partial thickness burn wound) comprising administering a therapeutically effective amount of a nanoemulsion to the burn wound. In some embodiments, administering the nanoemulsion to the burn wound preserves epithelial cells that line the shaft of each hair follicle within the burn wound. In further embodiments, the epithelial cells within the burn wound participate in re-epithelialization of the wound. Administration of nanoemulsion, in some embodiments, enhances proliferation of undamaged epithelial cells that line the shaft of each hair follicle within the burn wound. In further embodiments, administration of nanoemulsion suppresses neutrophil sequestration and/or activity. The invention is not limited by the amount of nanoemulsion utilized, the frequency of administration and/or the duration of administration. As described herein, administration of nanoemulsion provides therapeutic benefit to a burn wound upon application. In some embodiments, skin regeneration and wound healing takes place within minutes, hours, 1-2 days, 3-5 days, 1 week, 2 weeks, 3 weeks, 4 weeks, 5 weeks, 6 weeks, 7 weeks, or 8 weeks. In other embodiments, compositions and methods of the invention promote and/or induce skin regeneration and/or wound healing not possible (e.g., within any time frame) with conventional treatments heretofore available in the art. In some embodiments, administration of nanoemulsion reduces IL-1β expression within the burn wound. In some embodiments, administering reduces, attenuates and/or prevents bacterial growth at the burn wound site. In some embodiments, administering reduces tissue edema at the burn wound site. In some embodiments, administering reduces intravascular hypovolemia at the burn wound site.

The invention also provides methods of treating a burn wound comprising providing a subject harboring a burn wound; and a composition comprising a nanoemulsion; and administering a therapeutically effective amount of a composition comprising a nanoemulsion to the burn wound to prevent ischemic necrosis and protein denaturation at the burn wound site.

The invention also provides methods of treating a wound (e.g., any type of damage to the skin (e.g., dermis)) comprising administering a therapeutically effective amount of a composition comprising a nanoemulsion of the invention to the wound. In some embodiments, administration to the wound reduces the expression of one or more pro-inflammatory cytokines (e.g., IL-1, TNF-alpha, IL-6, IL-8, interferon gamma, or other pro-inflammatory cytokine) detectable at the wound. In some embodiments, a subject harboring a wound is seen by a physician or other medical care provider for the wound. For example, embodiments of the present disclosure provide methods of determining a treatment course of action and administering a nanoemulsion composition described herein. For example, in some embodiments, subjects with a wound (e.g., burn wound or other type of skin damage) are screened and/or tested (e.g., for inflammation, infection, wound severity, and/or other characteristic) by a physician or medical care provider and the results are used to determine a treatment course of action. For example, in some embodiments, subjects identified as having one or more types of wounds (e.g., a partial thickness burn wound, a cut, an abrasion, an infection (e.g., of the skin or hair follicles)) before beginning treatment are administered a composition comprising a nanoemulsion of the invention. In some embodiments, subjects found to not have one or more types of wounds are not administered a composition comprising a nanoemulsion of the invention. In some embodiments, a subject with a wound is screened for the presence or absence of one or more types of infection of the wound (e.g., bacterial, fungal, etc.). In some embodiments, subjects found to have a wound and/or infection of the wound is administered a composition comprising a nanoemulsion of the invention. In some embodiments, tests and/or assays for the presence or absence of the wound and/or infection of the wound are repeated (e.g., before, during or after treatment with a composition comprising a nanoemulsion of the invention). In some embodiments, tests/assays are repeated daily, weekly, monthly, annually, or less often.

The present invention is not limited by the type of nanoemulsion utilized for administration to a wound (e.g., a burn wound). Indeed, any nanoemulsion formulation described herein may be utilized. For example, the invention provides new nanoemulsion compositions (e.g., useful for the treatment of wounds (See, e.g., Examples 1-3)). In some embodiments, the nanoemulsion comprises a cationic surfactant, a nonionic surfactant, an alcohol (e.g., ethanol or glycerol), a chelating agent (e.g. EDTA), oil, and water, wherein the blend ratio of cationic surfactant to nonionic surfactant is between 6:1 and 1:48 (e.g., between 1:1 and 1:48, between 1:1 and 1:24, between 1:1.2 and 1:24, between 1:1.4 and 1:24, between 1:1.6 and 1:24, between 1:1.8 and 1:24, between 1:2 and 1:24, between 1:2 and 1:10, between 1:3 and 1:6, between 1:1.4 and 1:6), although lower and higher ratios may also be used. In a preferred embodiment, the blend ratio of cationic surfactant to nonionic surfactant is or is between 1:3 and 1:6.

The invention is not limited by the type of cationic surfactant. Indeed, any cationic surfactant with any size and type of cationic head group and varying tail chemistry and carbon chain lengths, as well as single chained and dual chained cationic surfactants and/or lipids, may be used including, but not limited to a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Similarly, the invention is not limited by the type of nonionic surfactant. Indeed, a number of nonionic surfactants may be used including, but not limited to an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxerms, Poloxamers (nonionic triblock copolymers, also known by the trade names Synperonics, Pluronics and Kolliphor, polyoxypropylene-polyoxyethylene copolymer type, P124®, P188®, P236®, P388®, and P407®) Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, a poloxamer, semi-synthetic derivatives thereof, or combinations thereof.

In some embodiments, the cationic surfactant is benzalkonium chloride and the nonionic surfactant is a polysorbate. In further embodiments, the cationic surfactant is benzalkonium chloride and the nonionic surfactant is polysorbate 20. In a preferred embodiment, the surfactant blend ratio of benzalkonium chloride to polysorbate 20 is 1:3 or 1:6. In some embodiments, the cationic surfactant is cetylpyridinium chloride and the nonionic surfactant is poloxamer 407. In a preferred embodiment, the blend ratio of cetylpyridinium chloride to poloxamer 407 is 1:6. The invention is not limited by the particle size of a nanoemulsion of the invention. In some embodiments, nanoemulsion formulations of the invention have an average particle (droplet) size of about 200 nm to about 600 nm. In more preferred embodiments, nanoemulsion formulations of the invention have an average particle (droplet) size of about 300 nm-400 nm, 325 nm-375 nm, 350 nm-370 nm, 360 nm, although smaller (e.g., less than about 300 nm) and larger (e.g., greater than 400 nm) particle sizes also find use in the compositions and methods described herein). In a preferred embodiment, nanoemulsion formulations of the invention undergoes high pressure processing in order to have a particle (droplet) size of about 200 nm-300 nm (e.g, ˜340 nm, 350 nm or 360 nm.

The invention is not limited by the way a nanoemulsion is administered to a burn wound. In some embodiments, a nanoemulsion is applied as a liquid (e.g., via a sprayer). In other embodiments, a nanoemulsion is administered as a cream. In still further embodiments, the nanoemulsion is administered via impregnating a wound dressing with the nanoemulsion and using the impregnated dressing to cover the wound.

In some embodiments, the invention provides compositions comprising nanoemulsion described herein. For example, in some embodiments, the invention provides a wound dressing, bandage and/or other type of wound covering impregnated with a nanoemulsion described herein. The invention is not limited by the amount of nanoemulsion utilized to impregnate a dressing, bandage and/or other type of wound covering. In a preferred embodiment, a dressing, bandage and/or other type of wound covering is impregnated with a therapeutically effective amount of nanoemulsion.

In further embodiments, the invention provides a composition for the treatment of a burn wound comprising a nanoemulsion comprising a cationic surfactant, a nonionic surfactant, an alcohol or humectant (e.g., ethanol or glycerol and/or combination), a chelating agent (e.g. EDTA), oil, and water, wherein the surfactant blend ratio of cationic surfactant to nonionic surfactant is 1:3 to 1:6 and a wound dressing. The invention is not limited to any particular type of wound dressing. Indeed, many types of wound dressings are known in the art and find use in the present invention.

In some embodiments, a nanoemulison composition of the invention comprises between 1-50% nanoemulsion solution, although greater and lesser amounts also find use in the invention. For example, in some embodiments, a nanoemulsion composition may comprise 1.0%-10%, about 10%-20%, about 20%-30%, about 30%-40%, about 40%-50%, about 50%-60% or more nanoemulsion. In some embodiments, the composition is stable (e.g., at room temperature (e.g., for 12 hours, one day, two days, three days, four days, a week, two weeks, three weeks, a month, two months, three months, four months, five months, six months, 9 months, a year or more. In some embodiments, the composition comprises a nanoemulsion comprising droplets the have an average diameter selected from the group comprising less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof.

In some embodiments, a nanoemulison composition of the invention comprises between 1-100% nanoemulsion cream, although greater and lesser amounts also find use in the invention. For example, in some embodiments, a nanoemulsion composition may comprise about 70%-100% or more nanoemulsion, preferably 80% nanoemulsion.

In some embodiments, a composition comprising a nanoemulsion of the invention further comprises an antimicrobial agent and/or anti-inflammatory agent. The present invention is not limited by the type of antimicrobial agent and/or anti-inflammatory agent utilized. Indeed, a variety of antimicrobial agents or an anti-inflammatory agents may be co-administered with the composition comprising a nanoemulsion including but not limited to those described herein. In some embodiments, the antimicrobial agent is an antibiotic. In some embodiments, the anti-inflammatory agent is silver nitrate (AgNO₃), silver sulfadiazine, mafenide acetate, nanocrystalline impregnated silver dressings, a p38 MAPK inhibitor or other anti-inflammatory agent. In some embodiments, the present invention provides a method of treating an infection present on and/or within a burn wound comprising administering the composition to the infection under conditions such that the composition kills, attenuates growth of and/or eliminates bacteria associated with the infection. The present invention is not limited by the type of bacteria associated with infection of a burn wound treated with a nanoemulsion of the invention. In some embodiments, bacteria associated with infection comprise Staphylococcus aureus. In some embodiments, the Staphylococcus aureus are antibiotic resistant. In some embodiments, the bacteria associated with the infection comprise Pseudomonas aeruginosa. The present invention is not limited by the type of burn wound treated. In some embodiments, the burn wound is a superficial burn wound, a partial thickness burn wound, or other type of burn wound. Compositions and methods of the invention find use in the treatment of a burn wounds caused by an event selected from a thermal insult, a chemical insult, an electrical insult, a friction-induced insult, and/or a UV radiation insult.

The present invention is not limited by the type of nanoemulsion utilized. Indeed, a variety of nanoemulsions are contemplated to be useful in the present invention. For example, in some embodiments, nanoemulsion utilized for burn wound treatment comprises an oil-in-water emulsion, the oil-in-water emulsion comprising a discontinuous oil phase distributed in an aqueous phase, a first component comprising a solvent (e.g., an alcohol or glycerol), and a second component comprising a surfactant or a halogen-containing compound. The aqueous phase can comprise any type of aqueous phase including, but not limited to, water (e.g., diH₂O, distilled water, tap water) and solutions (e.g., phosphate buffered saline solution). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In some preferred embodiments, the oil phase comprises 30-90 vol % of the oil-in-water emulsion (i.e., constitutes 30-90% of the total volume of the final emulsion), more preferably 50-80%. While the present invention in not limited by the nature of the alcohol component, in some preferred embodiments, the alcohol is ethanol, methanol or glycerol. Furthermore, while the present invention is not limited by the nature of the surfactant, in some preferred embodiments, the surfactant is a polysorbate surfactant (e.g., TWEEN 20, TWEEN 40, TWEEN 60, and TWEEN 80), a poloxamer (e.g., P407), a pheoxypolyethoxyethanol (e.g., TRITON X-100, X-301, X-165, X-102, and X-200, and TYLOXAPOL) or sodium dodecyl sulfate. Likewise, while the present invention is not limited by the nature of the halogen-containing compound, in some preferred embodiments, the halogen-containing compound comprises a cetylpyridinium halide, cetyltrimethylammonium halide, cetyldimethylethylammonium halide, cetyldimethylbenzylammonium halide, cetyltributylphosphonium halide, dodecyltrimethylammonium halide, tetradecyltrimethylammonium halide, cetylpyridinium chloride, cetyltrimethylammonium chloride, cetylbenzyldimethylammonium chloride, cetylpyridinium bromide, cetyltrimethylammonium bromide, cetyidimethylethylammonium bromide, cetyltributylphosphonium bromide, dodecyltrimethylammonium bromide, or tetrad ecyltrimethylammonium bromide. Nanoemulsions of the present invention may further comprise third, fourth, fifth, etc. components. In some preferred embodiments, an additional component is a surfactant (e.g., a second surfactant), a germination enhancer, a phosphate based solvent (e.g., tributyl phosphate), a neutramingen, L-alanine, ammonium chloride, trypticase soy broth, yeast extract, L-ascorbic acid, lecithin, p-hyroxybenzoic acid methyl ester, sodium thiosulate, sodium citrate, inosine, sodium hyroxide, dextrose, and polyethylene glycol (e.g., PEG 200, PEG 2000, etc.). In some embodiments, the oil-in-water emulsion comprises a quaternary ammonium compound. In some preferred embodiments, the oil-in-water emulsion has no detectable toxicity to plants or animals (e.g., to humans). In other preferred embodiments, the oil-in-water emulsion causes no detectable irritation to plants or animals (e.g., to humans). In some embodiments, the oil-in-water emulsion further comprises any of the components described above. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N, N-dimethyl-, chloride (or) Didecyl dimethyl ammonium chloride; 2-(2-(p-(Diisobuyl)cresosxy)ethoxy)ehyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl) benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C12-18); Di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis (2-hydroxyethyl) octyl hydrogen ammonium chloride; dodecyl dimethyl benzyl ammonium chloride; dodecylcarbamoyl methyl dinethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethyylbenzyl ammonium chloride; and n-octadecyl dimethyl benzyl ammonium chloride. In some embodiments, the emulsion lacks any antimicrobial substances (i.e., the only antimicrobial composition is the emulsion itself). In some embodiments, the nanoemulion comprises a poloxymer.

### BRIEF DESCRIPTION OF THE DRAWINGS

FIGS. 1A-C. (FIG. 1A) shows a bacterial wound infection model utilized during development of embodiments of the invention; (FIG. 1B) shows that topical application of 10% NB-402 (CPC/P407) inhibited Pseudomonas aeruginosa growth in burn wounds; and (FIG. 1C) shows that topical application of 10% NB-201(BAC/TWEEN 20) inhibited Staphylococcus aureus growth in burn wounds.

FIG. 2 shows that 10% NB-402 treatment after partial thickness burn injury and Pseudomonas aeruginosa infection decreased production of dermal proinflammatory cytokines.

FIG. 3 shows that 10% NB-402 treatment after partial thickness burn injury and Pseudomonas aeruginosa infection decreased dermal neutrophils sequestration as evidenced by myeloperoxidase assay.

FIG. 4 depicts quantitative wound culture results for Staphylococcus aureus.

FIG. 5 shows that 10% NB-201 and 10% NB-402 treatment after partial thickness burn injury and Staphylococcus aureus infection inhibited production of dermal proinflammatory cytokines.

FIG. 6 shows that 10% NB-201 and 10% NB-402 treatment after partial thickness burn injury and Staphylococcus aureus infection decreased dermal neutrophil sequestration as evidenced by myeloperoxidase assay.

FIGS. 7A-B show (FIG. 7A) a partial-thickness burn injury model utilized during development of embodiments of the invention; and (FIG. 7B) a photographic (Panels A-H) and cross-sectional histology (Panels I-L) analysis of burn skin after treatment with saline, 10% Placebo Vehicle (P407) or 10% NB-201 or 10% NB-402.

FIGS. 8A-B show (FIG. 8A) that topical application of NB-201 and NB-402 after partial thickness burn injury in the absence of infection decreases production of dermal pro-inflammatory cytokines and myeloperoxidase (MPO); (FIG. 8B) histopathology detailing neutrophil counts per slide. * p<0.05 vs. Saline, one-way ANOVA with Tukey's multiple comparison test. #p<0.05 vs. NE vehicle, one-way ANOVA with Tukey's multiple comparison test;

FIGS. 9A-B show that topical application of NB-201 and NB-402 after partial thickness burn injury in the absence of infection (FIG. 9A) decreased histology scores and (FIG. 9B) lead to maintained mean body mass versus controls (* p<0.05 vs. Saline, one-way ANOVA with Tukey's multiple comparison test. #p<0.05 vs. NE vehicle, one-way ANOVA with Tukey's multiple comparison test).

FIG. 10 shows histopathologic images from partial thickness burned skin after treatment with saline control. Total magnification was 40×, 100×, 200× and 400×.

FIG. 11 shows histopathologic images from partial thickness burned skin after treatment with saline control. Total magnification was 100× and 200×.

FIG. 12 shows histopathologic images from partial thickness burned skin after treatment with 10% NB-402+20 mM EDTA placebo control. Total magnification was 400× and 200×.

FIG. 13 shows histopathologic images from partial thickness burned skin after treatment with 10% NB-201+20 mM EDTA. Total magnification was 400×.

FIG. 14 depicts a graph showing a summary of pathologic scoring data compared using One-way Anova Kruskal-Wallis test (p=0.0941) followed by Dunn's Multiple Comparison test.

FIGS. 15A-B show (FIG. 15A) porcine burn wound progression and healing model utilized during development of embodiments of the invention; and (FIG. 15B) specific treatments utilized.

FIGS. 16A-C. show macroscopic burns healing time course. (FIG. 16A) burn sites created by application of copper bars pre-heated to 80° C. in water bath for 20 seconds. (FIG. 16B) burn sites created by application of copper bars pre-heated to 80° C. in water bath for 30 seconds. (FIG. 16C) pathology cross sectional histology skin samples stained with hematoxylin and eosin (H&E) (day 21).

FIGS. 17A-B show NB-201 suppressed burn induced soluble mediators production (FIG. 17A) within partial thickness wounds created by 80° C. heated blocks and applied to the skin for 20 seconds, and (FIG. 17B) within partial thickness wounds created by 80° C. heated blocks and applied to the skin for 30 seconds. Statistics: one-way ANOVA with Tukey's post-test. Bars: average±SD

FIG. 18 shows NB-201 controlled burn trauma associated infection. Statistics: one-way ANOVA with Tukey's post-test. Bars: average±SD.

FIG. 19 shows pathology/histology skin samples stained with H&E and analyzed by two independent pathologists. Score created by pathologists were averaged and plotted. Statistics: one-way ANOVA with Tukey's multiple comparison test. Bars: average±SD.

FIGS. 20A-B show NB-201 reduced neutrophil sequestration after skin burn. (FIG. 20A) MPO assay and histopathologic neutrophils count. (FIG. 20B) Shows representative histopathologic neutrophils count. Statistics: one-way ANOVA with Tukey's post-test. Bars: average±SD.

FIG. 21 shows NB-201 saved hair follicle cells proliferation. Representative microphotographs are shown of burned and control tissue stained for ki-67 to visualize fast proliferating cells.

FIG. 22 shows NB-201 treatment restored hair follicles on day 21 post burn. Crossectional skin histological samples stained with H&E and viable hair follicles were counted. Statistics: one-way ANOVA with Tukey's post-test. Bars: average±SD.

FIG. 23 shows IL-1β signaling cascade.

FIG. 24 depicts a schematic of burn wound progression/conversion in one embodiment of the invention.

FIG. 25 shows the effect of the surfactant blend ratio in three different CPC/Tween 20 formulations at a 1:6, 1:1, and 6:1 ratio, all containing 20 mM EDTA. Changing the surfactant blend ratio (cationic:nonionic) alters the positive surface charge density. All of the droplets retain an overall positive surface charge: a) illustrates a 1:6 surfactant blend ratio, b) 1:1 surfactant blend ratio, and c) 6:1 surfactant blend ratio.

FIG. 26 shows the effect of surfactant blend ratio (CPC/Tween 20) and bio-load (serum level) on mean particle size.

FIG. 27 shows the effect of surfactant blend ratio (CPC/Tween 20) and bio-load (serum level) on the polydispersity index (PdI).

FIG. 28 shows the effect of surfactant blend ratio (CPC/Tween 20) and bio-load (serum level) on the zeta potential.

FIG. 29 shows the effect of cationic surfactant (CPC or DODAC) and bio-load (serum level) on the mean particle size.

FIG. 30 shows the effect of cationic surfactant (CPC or DODAC) and bio-load (serum level) on the polydispersity index (PdI).

FIG. 31 shows the effect of cationic surfactant (CPC or DODAC) and bio-load (serum level) on the zeta potential.

FIG. 32 shows the effect of nonionic surfactant (Tween 20 or P407) and bio-load (serum level) on Mean particle size.

FIG. 33 shows the effect of nonionic surfactant (Tween 20 or P407) and bio-load (serum level) on polydispersity index (PdI).

FIG. 34 shows the effect of nonionic surfactant (Tween 20 or P407) and bio-load (serum level) on the zeta potential.

FIG. 35 shows the effect of bioloading with a 10% Benzalkonium Chloride/Tween 20 (1:3)+20 mM EDTA with respect to: Panel a) mean particle size (Z-average, nm), Panel b) Polydispersity Index (PdI), Panel c) zeta potential.

### DEFINITIONS

To facilitate an understanding of the present invention, a number of terms and phrases are defined below:

As used herein, the term “microorganism” refers to any species or type of microorganism, including but not limited to, bacteria, viruses, archaea, fungi, protozoans, mycoplasma, prions, and parasitic organisms. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism.

As used herein, the term “pathogen” refers a biological agent that causes a disease state (e.g., infection, sepsis, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are Gram-negative or Gram-positive. “Gram-negative” and “Gram-positive” refer to staining patterns with the Gram-staining process, which is well known in the art. (See e.g., Finegold and Martin, Diagnostic Microbiology, 6th Ed., CV Mosby St. Louis, pp. 13-15 (1982)). “Gram-positive bacteria” are bacteria that retain the primary dye used in the Gram stain, causing the stained cells to generally appear dark blue to purple under the microscope. “Gram-negative bacteria” do not retain the primary dye used in the Gram stain, but are stained by the counterstain. Thus, Gram-negative bacteria generally appear red. In some embodiments, bacteria are continuously cultured. In some embodiments, bacteria are uncultured and existing in their natural environment (e.g., at the site of a wound or infection) or obtained from patient tissues (e.g., via a biopsy). Bacteria may exhibit pathological growth or proliferation. Examples of bacteria include, but are not limited to, bacterial cells of a genus of bacteria selected from the group comprising Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, Thermoactinomycetes, Mycobacterium, Treponema, Borrelia, Leptospira, and Chlamydiae.

As used herein, the terms “microorganism” and “microbe” refer to any species or type of microorganism, including but not limited to, bacteria, archaea, fungi, protozoans, mycoplasma, and parasitic organisms.

As used herein, the term “fungi” is used in reference to eukaryotic organisms such as molds and yeasts, including dimorphic fungi.

As used herein the terms “disease” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms (e.g., diarrhea, nausea, fever, pain, blisters, boils, rash, immune suppression, inflammation, etc.) that are associated with any impairment of the normal state of a subject or of any of its organs or tissues that interrupts or modifies the performance of normal functions. A disease or pathological condition may be caused by or result from contact with a microorganism (e.g., a pathogen or other infective agent (e.g., a virus or bacteria)), may be responsive to environmental factors (e.g., malnutrition, industrial hazards, and/or climate), may be responsive to an inherent defect of the organism (e.g., genetic anomalies) or to combinations of these and other factors.

As used herein, the terms “burn,” “skin burn, “burn wound” and the like refer to a type of injury to flesh or skin caused by a thermal insult, chemical insult, electrical insult, friction-induced insult and/or UV radiation insult. Burns that affect only the superficial skin (epidermis) are known in the art as superficial or first-degree burns that can be characterized by clinical findings of redness, moderate pain and no blistering. When damage penetrates into some of the underlying layers (epidermis and the dermis are damaged), the burn is characterized as a partial-thickness or second-degree burn that can be characterized by clinical findings of blistering, epidermal and dermal damage and severe pain (epidermis and dermis are destroyed and there is subcutaneous tissue damage). In a full-thickness or third-degree burn, the injury extends to all layers of the skin (dermis, deep dermis, underlying tissue and possibly fascia bone or muscle). A fourth-degree burn involves injury to deeper tissues, such as muscle or bone.

“Respiratory” and “respiration” refer to the process by which oxygen is taken into the body and carbon dioxide is discharged, through the bodily system including the nose, throat, larynx, trachea, bronchi and lungs.

“Respiratory infection” and “pulmonary infection” refer to an infection (e.g., bacterial, viral, fungal, etc.) of the respiratory tract. In humans, the respiratory tract comprises the upper respiratory tract (e.g., nose, throat or pharynx, and larynx); the airways (e.g., voice box or larynx, windpipe or trachea, and bronchi); and the lungs (e.g., bronchi, bronchioles, alveolar ducts, alveolar sacs, and alveoli).

“Respiratory disease”, “pulmonary disease,” “respiratory disorder”, “pulmonary disorder,” “respiratory condition”, “pulmonary condition,” “pulmonary syndrome,” and “respiratory syndrome” refer to any one of several ailments that involve inflammation and affect a component of the respiratory system including especially the trachea, bronchi and lungs. Examples of such ailments include acute alveolar disease, obstructive respiratory disease (e.g., asthma; bronchitis; and chronic obstructive pulmonary disease, referred to as COPD), upper airway disease (e.g., such as otitis media, and rhinitis/sinusitis), insterstitial lung disease, allergy, and respiratory infection (e.g., pneumonia, pneyumocystis carinii, and respiratory syncitial virus (RSV)).

Specific examples of acute alveolar disease include acute lung injury (ALI), acute respiratory distress syndrome (ARDS), meconium aspiration syndrome (MAS) and respiratory distress syndrome (RDS). ALI is associated with conditions that either directly or indirectly injure the air sacs of the lung, the alveoli. ALI is a syndrome of inflammation and increased permeability of the lungs with an associated breakdown of the lungs' surfactant layer. The most serious manifestation of ALI is ARDS. Among the causes of ALI are complications typically associated with certain major surgeries, mechanical ventilator induced lung injury (often referred to as VILI), smoke inhalation, pneumonia, and sepsis.

The term “subject” as used herein refers to organisms to be treated by the compositions of the present invention. Such organisms include animals (domesticated animal species, wild animals), and humans.

As used herein, the terms “inactivating,” “inactivation” and grammatical equivalents, when used in reference to a microorganism refer to the killing, elimination, neutralization and/or reducing the capacity of the microorganism to infect and/or cause a pathological response and/or disease in a host.

As used herein, the term “fusigenic” is intended to refer to an emulsion that is capable of fusing with the membrane of a microbial agent (e.g., a bacterium or bacterial spore). Specific examples of fusigenic emulsions are described herein.

As used herein, the term “lysogenic” refers to an emulsion (e.g., a nanoemulsion) that is capable of disrupting the membrane of a microbial agent (e.g., a virus (e.g., viral envelope) or a bacterium, bacterial spore, or bacterial biofilm). In preferred embodiments of the present invention, the presence of a lysogenic and a fusigenic agent in the same composition produces an enhanced inactivating effect compared to either agent alone. Methods and compositions using this improved antimicrobial composition are described in detail herein.

The terms “nanoemulsion,” “emulsion,” and “water in oil emulsion” are used interchangeably herein to refer to dispersions or droplets, as well as other lipid structures that can form as a result of hydrophobic forces that drive apolar residues (e.g., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases.

As used herein, the terms “contact,” “contacted,” “expose,” and “exposed,” when used in reference to a nanoemulsion and a live microorganism, refer to bringing one or more nanoemulsions into contact with a microorganism (e.g., a pathogen) such that the nanoemulsion kill and/or attenuate growth of the microorganism or pathogenic agent, if present. The present invention is not limited by the amount or type of nanoemulsion used for microorganism killing and/or growth attenuation. The terms “contact,” “contacted,” “expose,” and “exposed,” when used in reference to burn wound refer to bringing one or more nanoemulsions into contact with a burn wound (e.g., a superficial burn wound, a partial thickness burn wound, a deep partial thickness burn wound or a full thickness burn wound). Ratios and amounts of nanoemulsion are contemplated in the present invention including, but not limited to, those described herein (e.g., in Example 1).

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “surfactant” refers to any molecule having both a polar head group, which energetically prefers solvation by water, and a hydrophobic tail which is not well solvated by water. The term “cationic surfactant” refers to a surfactant with a cationic head group. The term “anionic surfactant” refers to a surfactant with an anionic head group.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described by Meyers, (Meyers, Surfactant Science and Technology, VCH Publishers Inc., New York, pp. 231-245 [1992]), incorporated herein by reference. As used herein, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water which are good solubilizers of water in oils are at the low end of the scale.

As used herein the term “interaction enhancers” refers to compounds that act to enhance the interaction of an emulsion with a microorganism (e.g., with a cell wall of a bacteria (e.g., a Gram negative bacteria) or with a viral envelope. Contemplated interaction enhancers include, but are not limited to, chelating agents (e.g., ethylenediaminetetraacetic acid (EDTA), ethylenebis(oxyethylenenitrilo)tetraacetic acid (EGTA), and the like) and certain biological agents (e.g., bovine serum abulmin (BSA) and the like).

The terms “buffer” or “buffering agents” refer to materials which when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the term “effective amount” refers to the amount of a composition (e.g., a composition comprising a nanoemulsion) sufficient to effect a beneficial or desired result (e.g., to treat and/or prevent infection (e.g., through bacterial cell killing and/or prevention of bacterial cell growth). An effective amount can be administered in one or more administrations, applications or dosages and is not intended to be limited to a particular formulation or administration route.

As used herein, the term “adjuvant” refers to any substance that can stimulate an immune response (e.g., a mucosal immune response). Some adjuvants cause activation of a cell of the immune system (e.g., an adjuvant can cause an immune cell to produce and secrete a cytokine). Examples of adjuvants that can cause activation of a cell of the immune system include, but are not limited to, the nanoemulsion formulations described herein, saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); cholera toxin (CT), and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, immunogenic compositions described herein are administered with one or more adjuvants (e.g., to skew the immune response towards a Th1 and/or Th2 type response).

As used herein, the term “an amount effective to induce an immune response” (e.g., of a composition for inducing an immune response), refers to the dosage level required (e.g., when administered to a subject) to stimulate, generate and/or elicit an immune response in the subject. An effective amount can be administered in one or more administrations (e.g., via the same or different route), applications or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

As used herein, the term “immune response” refers to any detectable response by the immune system of a subject. For example, immune responses include, but are not limited to, an alteration (e.g., increase) in Toll receptor activation, lymphokine (e.g., cytokine (e.g., Th1 or Th2 type cytokines) or chemokine) expression and/or secretion, macrophage activation, dendritic cell activation, T cell (e.g., CD4+ or CD8+ T cell) activation, NK cell activation, and/or B cell activation (e.g., antibody generation and/or secretion). Additional examples of immune responses include binding of an immunogen (e.g., antigen (e.g., immunogenic polypeptide)) to an MHC molecule and induction of a cytotoxic T lymphocyte (“CTL”) response, induction of a B cell response (e.g., antibody production), and/or T-helper lymphocyte response, and/or a delayed type hypersensitivity (DTH) response (e.g., against the antigen from which an immunogenic polypeptide is derived), expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to an antigen and/or immunogen (e.g., both the initial response to an immunogen (e.g., a pathogen) as well as acquired (e.g., memory) responses that are a result of an adaptive immune response).

As used herein, the terms “purified” or “to purify” refer to the removal of contaminants or undesired compounds from a sample or composition. As used herein, the term “substantially purified” refers to the removal of from about 70 to 90%, up to 100%, of the contaminants or undesired compounds from a sample or composition.

As used herein, the terms “administration” and “administering” refer to the act of giving a drug, prodrug, or other agent, or therapeutic treatment (e.g., a composition of the present invention) to a physiological system (e.g., a subject or in vivo, in vitro, or ex vivo cells, tissues, and organs).

As used herein, the terms “co-administration” and “co-administering” refer to the administration of at least two agent(s) (e.g., a nanoemulsion and one or more other pharmaceutically acceptable substances (e.g., a second nanoemulsion)) or therapies to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent. In other embodiments, co-administration is preferable to treat and/or prevent infection by more than one type of infectious agent (e.g., bacteria and/or viruses).

As used herein, the term “topically” refers to application of a compositions of the present invention (e.g., a composition comprising a nanoemulsion) to the surface of the skin and/or mucosal cells and tissues (e.g., alveolar, buccal, lingual, masticatory, vaginal or nasal mucosa, and other tissues and cells which line hollow organs or body cavities). Compositions described herein can be applied using any pharmaceutically acceptable method, such as for example, intranasal, buccal, sublingual, oral, rectal, ocular, parenteral (intravenously, intradermally, intramuscularly, subcutaneously, intracisternally, intraperitoneally), pulmonary, intravaginal, locally administered, topically administered, mucosally administered, via an aerosol, or via a buccal or nasal spray formulation. Further, the nanoemulsion vaccines described herein can be formulated into any pharmaceutically acceptable dosage form, such as a liquid dispersion, gel, aerosol, pulmonary aerosol, nasal aerosol, ointment, cream, semi-solid dosage form, and a suspension. Further, the composition may be a controlled release formulation, sustained release formulation, immediate release formulation, or any combination thereof.

The terms “pharmaceutically acceptable” or “pharmacologically acceptable,” as used herein, refer to compositions that do not substantially produce adverse allergic or immunological reactions when administered to a host (e.g., an animal or a human). Such formulations include dips, sprays, seed dressings, stem injections, sprays, and mists. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like. Examples of carriers, stabilizers and adjuvants have been described and are known in the art (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a composition of the present invention that is physiologically tolerated in the target subject. “Salts” of the compositions of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compositions of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compositions of the present invention are contemplated as being pharmaceutically acceptable. However, salts of acids and bases that are non-pharmaceutically acceptable may also find use, for example, in the preparation or purification of a pharmaceutically acceptable composition.

“Pulmonary application” and “pulmonary administration” refers to any means of applying a composition of the present invention to the pulmonary system of a subj et. The present invention is not limited to any particular means of administration. Indeed, a variety of means are contemplated to be useful for pulmonary administration including those described herein.

As used herein, the term “kit” refers to any delivery system for delivering materials. In the context of the nanoemulsion compositions of the present invention, such delivery systems include systems that allow for the storage, transport, or delivery of the compositions and/or supporting materials (e.g., written instructions for using the materials, etc.) from one location to another. For example, kits include one or more enclosures (e.g., boxes) containing the relevant nanoemulsions and/or supporting materials. As used herein, the term “fragmented kit” refers to delivery systems comprising two or more separate containers that each contain a subportion of the total kit components. The containers may be delivered to the intended recipient together or separately. For example, a first container may contain a composition comprising a nanoemulsion for a particular use, while a second container contains a second agent (e.g., an antibiotic or spray applicator). Indeed, any delivery system comprising two or more separate containers that each contains a subportion of the total kit components are included in the term “fragmented kit.” In contrast, a “combined kit” refers to a delivery system containing all of the components of a composition needed for a particular use in a single container (e.g., in a single box housing each of the desired components). The term “kit” includes both fragmented and combined kits.

### DETAILED DESCRIPTION OF THE INVENTION

The present invention relates to therapeutic nanoemulsion compositions and to methods of utilizing the same to treat a burn wound. In particular, nanoemulsion compositions are described herein that find use in reducing and/or preventing progression/conversion of a partial thickness burn wound (e.g., to deep partial thickness wound or a full thickness burn wound (e.g., by accelerating and/or improving burn wound healing)). Compositions and methods of the present invention find use in, among other things, clinical (e.g. therapeutic and preventative medicine), industrial, and research applications.

Contemporary burn wound management involves early debridement and reconstruction of non-viable skin coupled with provision of supportive care and topical antimicrobial dressing changes to partial thickness burn wounds. The goal of modern burn wound care is to provide an optimal environment for epidermal renewal. Restoration of skin integrity takes place via regrowth of keratinocytes from preserved hair follicles or transfer of split thickness skin grafts harvested from non-burn regions. During the period of epidermal renewal it is important to avoid further injury to the skin, abrogate burn wound progression, and minimize secondary complications such as wound infection. Early excision of full-thickness burn eschar, immediate skin grafting, and treatment of remaining open or partial thickness areas of burn wound with topical antimicrobial agents has heretofore been the most effective way of minimizing burn wound colonization and invasive wound infection. (See, e.g., Bessey, Wound care. In Herndon DN, ed: Total Burn Care 3^{rd} edition. Philadelphia, Pa.: Elsevier Inc., 2007, pp 127-135.). Popular topical antimicrobial agents include silver sulfadiazine (SILVADENE), mafenide acetate (SULFAMYLON), and colloidal silver impregnated dressings (ACTICOAT, SILVERLON). Each of these agents has potential limitations such as variable ability to penetrate eschar, uneven efficacy against both Gram-negative and Gram-positive bacteria, and potential toxicity to host immune cells (See, e.g., Steinstraesser et al., Antimicrob Agents Chemother 46(6):1837-1844, 2002).

The invention is not limited by the type of burn wound that can be treated using the compositions and methods described herein. Indeed, any burn wound of the flesh or skin caused by a thermal insult, chemical insult, electrical insult, friction-induced insult and/or UV radiation insult can be treated. Burn wounds are classified based upon a number of criteria. Burns that affect only the superficial skin (epidermis) are known as superficial or first-degree burns that can be characterized by clinical findings of redness, moderate pain and no blistering. When damage penetrates into some of the underlying layers (epidermis and the dermis are damaged), the burn is characterized as a partial-thickness or second-degree burn that can be characterized by clinical findings of blistering, epidermal and dermal damage and severe pain (epidermis and dermis are destroyed and there is subcutaneous tissue damage). In a full-thickness or third-degree burn, the injury extends to all layers of the skin (dermis, deep dermis, underlying tissue and possibly fascia bone or muscle). A fourth-degree burn involves injury to deeper tissues, such as muscle or bone.

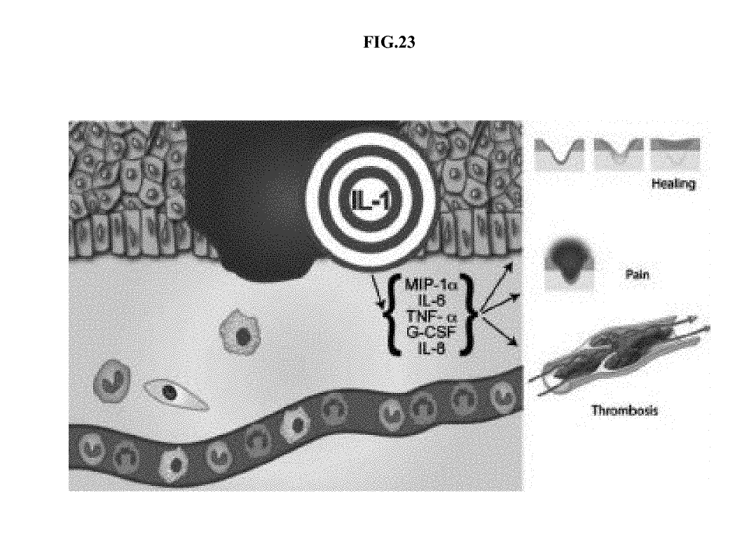

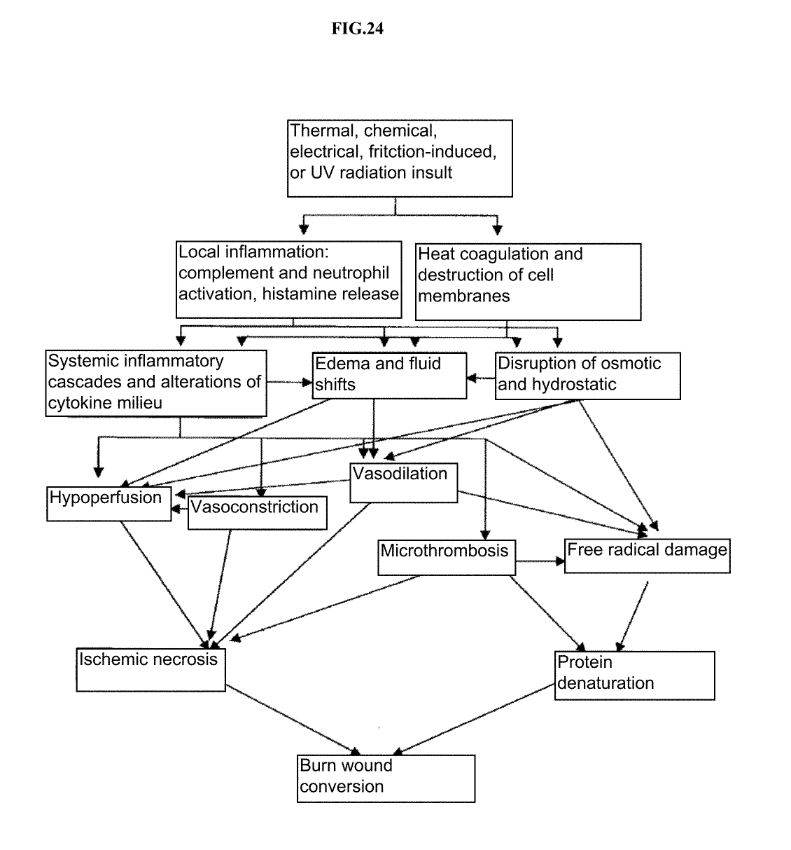

Burn wound progression (also known as conversion) is a process in which certain superficial partial-thickness burns spontaneously advance into deep partial-thickness or full-thickness wounds. Progression of an injury into deeper tissue is an important phenomenon in the treatment of thermal injury due to the fact that burn wound depth may be a significant determinant of morbidity and treatment. The depth of burn wounds is not entirely static, and multiple factors, each acting via a variety of pathophysiologic mechanisms, can promote the deepening of a burn wound. In a subacute time frame of 3-5 days, burns originally assessed to be superficial partial thickness have been observed to progress into deep partial-thickness or full-thickness burns (See, e.g., Kao and Garner, Plast Reconstr Surg. 2000; 105:2482-2492). This process of progressive damage to initially unburned tissue surrounding a burn wound is referred to as burn wound progression/conversion. A schematic of burn wound conversion/progression is shown in FIG. 23 (See, e.g., Singh et al., Annals of Plastic Surgery, 59(1): 109-115 (2007)).

The present invention provides nanoemulsion compositions and methods of using the same for the treatment of burn wounds. For example, as shown in the Examples, the present invention provides nanoemulsion compositions and methods of using the same to reduce, attenuate and/or prevent burn wound conversion/progression (e.g., from a partial thickness burn wound to a deep partial thickness burn wound or a full thickness burn wound).

For example, in a preferred embodiment, the invention provides novel nanoemulsion formulations (e.g. described in Examples 1-5) that significantly reduce, limit and/or ameliorate tissue injury in burn wounds (e.g., partial thickness burn wounds (thereby preventing the progression/conversion of a partial thickness burn wound into a deep partial thickness burn wound and/or a full thickness burn wound)) while concurrently and substantially reducing bacterial growth (e.g., of Pseudomonas aeruginosa and/or Staphylococcus aureus in a partial thickness burn (See Examples 4 and 5)). Reduction in the level of wound infection was associated with an attenuation of the local dermal inflammatory response (IL-1β and IL-6) and diminished neutrophil sequestration. Cross sectional histology of burned skin demonstrated a reduction in infiltration of inflammatory cells into the burned skin treated with nanoemulsion (e.g., NB-201 or NB-402) compared to saline treated controls. The burned skin of saline and placebo treated rats and pigs demonstrated accentuated fibrosis and granulation tissue formation, while rats and pigs treated with nanoemulsion (e.g., NB-201 or NB-402) had minimal gross evidence of burn wound progression. Histological analysis revealed a loss of epidermis in the saline and placebo treated groups, with intact epidermis in nanoemulsion (e.g., NB-201 and NB-402) treated groups.

Thus, the invention provides nanoemulsion compositions and methods utilizing nanoemulsion formulations described herein (e.g., for nanoemulsion therapy) to reduce, limit and/or ameliorate tissue injury in partial thickness burn wounds (e.g., thereby preventing the progression/conversion of a partial thickness burn wound into a deep partial thickness burn wound and/or a full thickness burn wound). In other embodiments, the present invention provides compositions and methods utilizing nanoemulsion formulations described herein (e.g., for nanoemulsion therapy) to reduce or prevent bacterial counts and inflammation associated with tissue injury in partial thickness burn wounds. As described herein, compositions and methods of the invention promote and/or induce skin regeneration and/or wound healing not possible (e.g., within any time frame) with conventional treatments heretofore available in the art. For example, in one embodiment, compositions and methods of the invention reduce and/or inhibit scarring associated with a wound (e.g., injury of the skin (e.g., burn wound (e.g., that is not made possible using conventional treatments (e.g., SILVADENE))) (See Examples 4 and 5). In another embodiment, compositions and methods of the invention reduce and/or inhibit scarring associated with wounds not caused by burn injury. For example, in some embodiments, the invention provides compositions and methods that reduce scarring from cuts, abrasions, acne, or other disturbance to the skin (e.g., dermis).

Thus, in some embodiments, the invention provides a prophylactic and/or therapeutice treatment that specifically limits burn wound progression while also acting as an antimicrobial agent. In some embodiments, the compositions and methods described herein are used to treat and/or inhibit growth of antibiotic resistant bacteria. Accordingly, in some embodiments, the present invention provides compositions and methods utilizing nanoemulsion formulations described herein (e.g., for nanoemulsion therapy) as a wound treatment to limit the conversion of partial-thickness burns to full-thickness injury while controlling bacterial super-infection.

While an understanding of a mechanism of action is not needed to practice the present invention and while the present invention is not limited to any particular mechanism, in some embodiments, compositions and methods of the invention inhibit burn wound progression/conversion via promoting skin regeneration (See, e.g., Example 5). While an understanding of a mechanism of action is not needed to practice the present invention and while the present invention is not limited to any particular mechanism, in some embodiments, compositions and methods of the invention inhibit burn wound progression/conversion via altering cytokine profile changes (e.g., local (e.g., at the site of the burn wound) and/or systemic cytokine profile changes) in the subject (See, e.g., Examples 4 and 5). Examples of cytokine profiles that are altered utilizing compositions and methods of the invention include, but are not limited to, IL-1β, IL-6, TNFα, CXCL1 and CXCL2. Use of the compositions and methods of the invention to alter cytokine profiles can be used to inhibit the inflammatory cascade (e.g., local and/or systemic inflammatory signaling) in a subject (See Examples 4 and 5). In addition, compositions and methods of the invention can be utilized to prevent, inhibit and/or reduce local inflammation, complement and neutrophil activation as well as histamine release (See, e.g., Examples 4 and 5).

While an understanding of a mechanism of action is not needed to practice the present invention and while the present invention is not limited to any particular mechanism, in some embodiments, compositions and methods of the invention inhibit burn wound progression/conversion via promoting hair follicle growth (See, e.g., Example 5). Use of the compositions and methods of the invention to promote hair follicle growth may may also promote skin growth and/or regeneration and/or maintenance post burn injury (See, e.g., Examples 4 and 5).

In some embodiments, compositions and methods of the invention are used to reduce and/or prevent pain associated with burn wounds. While an understanding of a mechanism of action is not needed to practice the present invention and while the present invention is not limited to any particular mechanism, in some embodiments, compositions and methods of the invention inhibit, reduce, and/or prevent pain associated with burn wounds via inhibition of IL-1β expression.

Compositions and methods of the invention prevent, inhibit and/or reduce heat coagulation and destruction of cell membranes (See Examples 4 and 5). Compositions and methods of the invention reduced and/or inhibited edema and/or fluid shifts that occur in response to burn injury. Compositions and methods of the invention also reduced and/or inhibited the disruption of osmotic hydrostatic gradients that occur in response to burn injury. Thus, compositions and methods of the invention can be utilized to maintain normal osmotic and hydrostatic gradients at the site of a burn wound. Compositions and methods of the invention also inhibited and/or prevented ischemic necrosis and protein denaturation associated with burn wounds (See Examples 4 and 5). Thus, compositions and methods of the invention can be utilized to prevent ischemic necrosis and protein denaturation associated with burn wounds (e.g., thereby inhibiting progression/conversion of a partial thickness burn wound to a full thickness burn wound).

Experiments conducted during development of embodiments of the invention identified and characterized new nanoemulsion formulations effective at treating burn wounds (e.g., the inhibition of and/or reduction of burn wound conversion/progression (See, e.g., Examples 1-5)).

Although an understanding of a mechanism of action is not needed to practice the present invention, and the present invention is not limited to any particular mechanism of action, in some embodiments, a nanoemulsion composition of the invention that is applied to a wound following burn injury is able to penetrate more deeply and uniformly into a burn wound (e.g., thereby keeping the epidermis intact, reducing necrotic inflammation and/or reducing dermal necrosis (e.g., in a dose-dependent manner)). In a preferred embodiment, compositions and methods of the invention are used to accelerate the proliferation of undamaged epithelial cells that line the shaft of each hair follicle, thereby increasing skin regeneration after a burn wound (e.g., See Example 5).

As shown in Examples 3,4 and 5, the present invention also provides methods of reducing, inhibiting and/or eliminating bacterial growth in a burn wound comprising providing a burn wound and a nanoemulsion and administering the nanoemulsion to the burn wound under conditions that bacterial growth is reduced, inhibited and/or eliminated. Compositions and methods of the invention find great utility in the prevention and treatment of microbial infection of burn wounds due to the fact that the antimicrobial mechanism of action of the compositions described herein are unlikely to lead to the development of microbial resistance. In some embodiments, a nanoemulsion composition described herein is combined with one or more antimicrobial drugs for administration to a burn wound to minimize bacterial growth at the burn wound site. The present invention is not limited to any particular antimicrobial drug. Indeed, any antimicrobial drug that inhibits bacterial growth known to those in the art can be utilized in combination with a nanoemulsion composition described herein.

In addition to local effects, severe dermal burns are known to induce the systemic inflammatory response syndrome (SIRS), which results in a high-risk of end-organ dysfunction (See, e.g., Barton et al., J Burn Care Rehabil 18(1):1-9, 1997). Increased vascular permeability and systemic capillary leak as a consequence of SIRS following burn injury creates seepage of plasma into interstitial tissue throughout the body. This tissue edema and intravascular hypovolemia is responsible for a host of undesired clinical problems such as shock, pulmonary dysfunction, abdominal or extremity compartment syndrome, and cardiac failure.

Accordingly, in some embodiments, and as shown in Examples 4 and 5, nanoemulsion compositions described herein can be administered to a burn wound to treat (e.g., reduce, attenuate and/or prevent) inflammation, tissue edema and/or intravascular hypovolemia at the site of a burn wound. Thus, in another preferred embodiment, compositions and methods of the invention are used to inhibit edema and/or fluid shifts that occur in response to burn injury. In some embodiments, reducing inflammation, tissue edema and/or intravascular hypovolemia at the site of a burn wound reduces the occurrence of shock, pulmonary dysfunction, abdominal or extremity compartment syndrome, and/or cardiac failure. In some embodiments, a nanoemulsion composition described herein is used in combination with (e.g., is co-administered with) one or more anti-inflammatory drugs to minimize early burn wound inflammation and tissue edema. The present invention is not limited to any particular anti-inflammatory drug. Indeed, any anti-inflammatory drug that minimizes early burn wound inflammation and tissue edema can be utilized in combination with a nanoemulsion composition described herein.

In some embodiments, nanoemulsion formulations described herein (e.g., NB-201 and/or NB-402) is administered to a burn wound to prevent, attenuate and/or eradicate bacterial growth (e.g., Staphylococcus aureus, P. aeruginosa, or other bacteria) within a burn wound (e.g, a partial thickness burn wound). Examples 1-5 show that reduction in microbial infection was coupled with generation of lower levels of local dermal pro-inflammatory cytokines and evidence of reduced neutrophil sequestration into the burn wound. This decrease in burn wound bacterial growth and inflammation also produced less capillary leak in the early post-thermal injury time-period. Having the ability to clinically reduce capillary leak and tissue edema in the immediate post-burn time-period provides a lesser need for large volume crystalloid fluid resuscitation and a reduction in the associated sequela of physiologic volume overload, pulmonary dysfunction, and abdominal compartment syndrome.

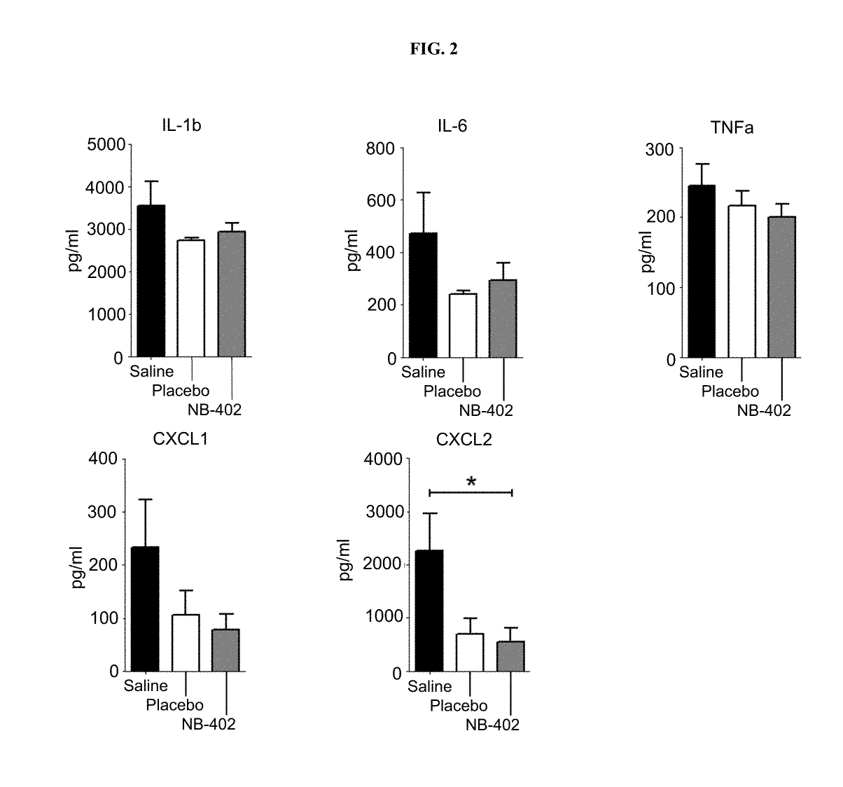

Skin that is damaged by thermal injury loses its ability to protect the host against infection from both the loss of physical barrier function and the secondary immunosuppression caused by the thermal injury. Moreover, increased production of TGF-β and IL-10 during the post-burn period can result in immunosuppression. (See, e.g., Lyons et al., Arch Surg 134(12):1317-1323, 1999; Varedi et al., Shock 16(5):380-382, 2001). It has been established that treatment of burn injured animals with anti-TGF-β can improve local and systemic clearance ofP. aeruginosa (See, e.g., Huang et al., J Burn Care Res 27(5):682-687, 2006). Inhibition of TGF-β also results in increased survival following bacterial challenge. As shown in FIGS. 2, 5, 8 and 25, nanoemulsion formulations of the invention can be used to alter cytokine expression in the context of a burn wound.

Onset of a bacterial infection within a burn wound can delay or even reverse the tissue healing process (See, e.g., Steinstraesser et al., Crit Care Med 29(7):1431-1437, 2001). Topical antimicrobial therapy is used to reduce the microbial load in the burn wound and reduce this risk of infection. Conventional topical agents have heretofore included silver nitrate (AgNO₃), silver sulfadiazine, mafenide acetate, and nanocrystalline impregnated silver dressings. Silver nitrate is limited in its use because of the problem it creates from contact staining and its limited antifungal activity. Silver sulfadiazine is the mainstay of topical burn antimicrobial treatment. It is bactericidal against P. aeruginosa and other Gram-negative enteric bacteria. Resistance to Silvadene by some of these organisms has emerged (See, e.g., Silver et al., J Ind Microbiol Biotechnol 33(7):627-634, 2006). The agent has limited antifungal activity, but can be used in conjunction with nystatin. Silvadene has no real ability to penetrate burn eschar and sometimes leads to leukopenia which requires conversion to another topical agent. The use of mafenide acetate is narrowed by the fact that it is bacteriostatic against select organisms, it has limited activity against Gram-positive bacteria such as Staphylococcus aureus, and that its use over a large surface area can lead to a metabolic acidosis because of its metabolism into a carbonic anhydrase inhibitor. The nanocrystalline silver dressings have the broadest activity against burn wound pathogens of the current agents available. They have a modest ability to penetrate eschar and can be left in place for many days (See, e.g., Church et al., Clin Microbiol Rev 19(2):403-434, 2006). SB 202190, an inhibitor of activated p38 MAPK, can substantially reduce the dermal inflammation generated in burn wounds (See, e.g., Arbabi et al., Shock. 26(2):201-209, 2006). Thermal injury initiates dermal inflammatory and pro-apoptotic cell signaling.

As shown in Examples 1-5, topical application of nanoemulsion formulations of the invention resulted in reduced hair follicle cell apoptosis within the dermis of burned skin. Thus, in some embodiments, the present invention provides nanoemulsion compositions that are utilized to reduce, when administered to a burn wound, conversion of the partial thickness burn wound within the “zone of stasis” to regions of full thickness burn.

In patients without evidence of inhalational injury, the burn wound itself is the primary source triggering the systemic inflammatory response via generation of pro-inflammatory cytokines and sequestration of neutrophils into the burn wound (See, e.g., Hansbrough et al., J Surg Res 61(1):17-22, 1996; Piccolo et al., Inflammation 23(4):371-385, 1999; Till et al., J Clin Invest 69(5):1126-1135, 1982). Topical application of a p38 MAPK inhibitor can control the source of inflammation at the level of the dermis, resulting in lower levels of pro-inflammatory mediators, reduced neutrophil sequestration and microvascular damage, and less epithelial apoptosis in burn wound hair follicle cells (See, e.g., Ipaktchi et al., Shock. 26(2):201-209, 2006). Dermal source control of inflammation also reduces bacterial growth and attenuates the systemic inflammatory response resulting in less acute lung injury and cardiac dysfunction following partial thickness burn injury in a rodent model. Accordingly, in some embodiments, a nanoemulsion of the invention is utilized (e.g., administered) alone or in combination with an anti-inflammatory and/or antimicrobial agent to reduce local dermal inflammation and risk of infection within burn wounds (e.g., partial thickness wounds, full thickness wounds or other burn wounds). The present invention is not limited by the type of anti-inflammatory agent and/or antimicrobial utilized for co-administration with a nanoemulsion described herein. Indeed, a variety of anti-inflammatory agents and/or antimicrobial agents can be used including, but not limited to, silver nitrate (AgNO₃), silver sulfadiazine, mafenide acetate, nanocrystalline impregnated silver dressings, p38 MAPK inhibitor (e.g., SB 202190), or another anti-inflammatory or antimicrobial agent described herein.

In some embodiments, when a nanoemulsion of the invention is administered to a burn wound, the nanoemulsion can be administered (e.g., to a subject (e.g., to a burn or wound surface)) by multiple methods, including, but not limited to, direct use or being suspended in a solution (e.g., colloidal solution) and applied to a surface (e.g., a surface comprising bacteria (e.g., pathogenic bacteria) or susceptible to bacterial invasion); being sprayed onto a surface using a spray applicator; being mixed with fibrin glue and applied (e.g., sprayed) onto a surface (e.g., skin burn or wound); being impregnated onto a wound dressing or bandage and applying the bandage to a surface (e.g., an infection or burn wound); being applied by a controlled-release mechanism; or being impregnated on one or both sides of an acellular biological matrix that is then placed on a surface (e.g., skin burn or wound) thereby protecting at both the wound and graft interfaces. In some embodiments, the invention provides a pharmaceutical composition containing (a) a composition comprising a nanoemulsion formulation described herein and (b) one or more other agents (e.g., an antibiotic). Examples of antibiotics include, but are not limited to, almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, Pep5, RP 59500, and TD-6424. In some embodiments, two or more combined agents (e.g., a composition comprising a nanoemulsion and an antibiotic) may be used together or sequentially. In some embodiments, an antibiotic may comprise bacteriocins, type A lantibiotics, type B lantibiotics, liposidomycins, mureidomycins, alanoylcholines, quinolines, eveminomycins, glycylcyclines, carbapenems, cephalosporins, streptogramins, oxazolidonones, tetracyclines, cyclothialidines, bioxalomycins, cationic peptides, and/or protegrins. In some embodiments, the composition comprises lysostaphin.

The present invention is not limited by the type of nanoemulsion utilized. Indeed, a variety of nanoemulsion formulations described herein are useful as or in the compositions and methods of the present invention.

For example, in some embodiments, a nanoemulsion comprises (i) an aqueous phase; (ii) an oil phase; and at least one additional compound. In some embodiments of the present invention, these additional compounds are admixed into either the aqueous or oil phases of the composition. In other embodiments, these additional compounds are admixed into a composition of previously emulsified oil and aqueous phases. In certain of these embodiments, one or more additional compounds are admixed into an existing emulsion composition immediately prior to its use. In other embodiments, one or more additional compounds are admixed into an existing emulsion composition prior to the compositions immediate use.

Additional compounds suitable for use in a nanoemulsion of the present invention include, but are not limited to, one or more organic, and more particularly, organic phosphate based solvents, surfactants and detergents, cationic halogen containing compounds, germination enhancers, interaction enhancers, food additives (e.g., flavorings, sweeteners, bulking agents, and the like) and pharmaceutically acceptable compounds. Certain exemplary embodiments of the various compounds contemplated for use in the compositions of the present invention are presented below. Unless described otherwise, nanoemulsions are described in undiluted form.

Stability on storage and after application of nanoemulsion formulations of the invention. Nanoemulsion formulations described herein are stable at about 40° C. and about 75% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment of the invention, the nanoemulsions of the invention can be stable at about 25° C. and about 60% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

Further, the nanoemulsions of the invention can be stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

The nanoemulsion formulations of the invention are stable upon application, as surprisingly the nanoemulsions do not lose their physical structure upon application. For example, as shown in Example 2, components of the nanoemulsion formulations expected to react with materials used to apply the nanoemulsions (e.g., bandages and dressings) in fact did not react/bind. In fact, there was no binding of BAK, CPC or EDTA to the TELFA pad (See Example 2). Microscopic examination of skin surface following application of a nanoemulsion according to the invention demonstrated the physical integrity of the nanoemulsions of the invention. While an understading of a mechanism is not needed to practice the present invention, and while the present invention is not limited to any particular mechanism, in some embodiments, physical integrity results in the desired absorption observed with the nanoemulsions of the invention.

Nanoemulsions

The term “nanoemulsion”, as defined herein, refers to a dispersion or droplet or any other lipid structure. Typical lipid structures contemplated in the invention include, but are not limited to, unilamellar, paucilamellar and multilamellar lipid vesicles, micelles and lamellar phases.

The nanoemulsion of the present invention comprises droplets having an average diameter size of less than about 1,000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 100 nm and less than or equal to about 400 nm. In a different embodiment, the droplets have an average diameter size greater than about 200 nm or greater than about 300 nm, and less than or equal to about 400 nm. In other embodiments of the invention, the nanoemulsion droplets have an average diameter of from about 300 nm to about 400 nm; or the nanoemulsion droplets have an average diameter of from about 350 nm to about 370 nm.

In one embodiment of the invention, the nanoemulsion has a narrow range of MIC (minimum inhibitory concentration) and MBC (minimum bactericidal concentrations) values. In another embodiment, the MIC and MBC for the nanoemulsion differ by less than or equal to four-fold, meaning that the nanoemulsion is bactericidal. In addition, the MIC and MBC for the nanoemulsion may differ by greater than four-fold, meaning that the nanoemulsion is bacteriostatic.

In one embodiment of the invention, the nanoemulsion comprises: (a) an aqueous phase; (b) about 1% oil to about 80% oil; (c) about 0.1% to about 50% organic solvent; (d) about 0.001% to about 10% surfactant or detergent; (e) about 0.0005% to about 1.0% of a chelating agent; or (e) any combination thereof. In another embodiment of the invention, the nanoemulsion comprises: (a) about 10% to about 80% oil; (b) about 1% to about 50% organic solvent; (c) at least one non-ionic surfactant present in an amount of about 0.1% to about 10%; (d) at least one cationic agent present in an amount of about 0.01% to about 3%; or any combination thereof.

In another embodiment, the nanoemulsion comprises a cationic surfactant which is either cetylpyridinium chloride (CPC) or benzalkonium chloride, or alkyl dimethyl benzyl ammonium chloride (BTC 824), or combination thereof. The cationic surfactant may have a concentration in the nanoemulsion of less than about 5.0% and greater than about 0.001%, or further, may have a concentration of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%.

In a further embodiment, the nanoemulsion comprises a non-ionic surfactant, and may have a concentration of about 0.01% to about 10.0%, or about 0.1% to about 3% of a non-ionic surfactant, such as a polysorbate.

In yet other embodiments of the invention, the nanoemulsion: (a) comprises at least one cationic surfactant; (b) comprises a cationic surfactant which is either cetylpyridinium chloride or benzalkonium chloride, or alkyl dimethyl benzyl ammonium chloride (BTC 824), or combination thereof; (c) comprises a cationic surfactant, and wherein the concentration of the cationic surfactant is less than about 5.0% and greater than about 0.001%; (d) comprises a cationic surfactant, and wherein the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%; or (e) any combination thereof. In yet other embodiments, (a) the nanoemulsion comprises at least one cationic surfactant and at least one non-cationic surfactant; (b) the nanoemulsion comprises at least one cationic surfactant and at least one non-cationic surfactant, wherein the non-cationic surfactant is a nonionic surfactant; (c) the nanoemulsion comprises at least one cationic surfactant and at least one non-cationic surfactant, wherein the non-cationic surfactant is a polysorbate nonionic surfactant; (d) the nanoemulsion comprises at least one cationic surfactant and at least one non-cationic surfactant, wherein the non-cationic surfactant is a nonionic surfactant, and the non-ionic surfactant is present in a concentration of about 0.05% to about 10%, about 0.05% to about 7.0%, about 0.1% to about 7%, or about 0.5% to about 5%; (e) the nanoemulsion comprises at least one cationic surfactant and at least one a nonionic surfactant, wherein the cationic surfactant is present in a concentration of about 0.05% to about 2% or about 0.01% to about 2%; or (0 any combination thereof.

In other embodiments, the nanoemulsion comprises: (a) water; (b) ethanol or glycerol (glycerine), or a combination thereof; (c) either cetylpyridinium chloride (CPC), or benzalkonium chloride, or alkyl dimethyl benzyl ammonium chloride (BTC 824), or a combination thereof (c) soybean oil; and (e) Poloxamer 407, Tween 80, or Tween 20. The nanoemulsion can further comprise EDTA.

In preferred embodiments, the invention provides a nanoemulsion described in Examples 1-5. In other preferred embodiments, the invention provides a nanoemulsion composition identified utilizing compositions and methods of identifying and characterizing nanoemulsions useful for the treatment of burn wounds described herein (See, e.g., Examples 1-5).

Quantities of each component present in the nanoemulsion refer to a therapeutic nanoemulsion, and not to a nanoemulsion to be tested in vitro. This is significant, as nanoemulsions tested in vitro generally have lower concentrations of oil, organic solvent, surfactant or detergent, and (if present) chelating agent than that present in a nanoemulsion intended for therapeutic use, e.g., topical use. This is because in vitro studies do not require the nanoemulsion droplets to traverse the skin. For topical, aerosol, intradermal etc. use, the concentrations of the components must be higher to result in a therapeutic nanoemulsion. However, the relative quantities of each component used in a nanoemulsion tested in vitro are applicable to a nanoemulsion to be used therapeutically and, therefore, in vitro quantities can be scaled up to prepare a therapeutic composition, and in vitro data is predictive of topical application success.

1. Aqueous Phase

The aqueous phase can comprise any type of aqueous phase including, but not limited to, water (e.g., H₂O, distilled water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate buffered saline (PBS). The aqueous phase may further be sterile and pyrogen free.

2. Organic Solvents

Organic solvents in the nanoemulsions of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

3. Oil Phase

The oil in the nanoemulsion of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

4. Surfactants/Detergents

The surfactant or detergent in the nanoemulsion of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a nonpolar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N, N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl) benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis (2-hydroxyethyl) octyl hydrogen ammonium chloride, Dodecyl dimethyl benzyl ammonium chloride, Dodecylcarbamoyl methyl dinethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with a particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the nanoemulsion comprises a cationic surfactant, which can be cetylpyridinium chloride. In other embodiments of the invention, the nanoemulsion comprises a cationic surfactant, and the concentration of the cationic surfactant is less than about 5.0% and greater than about 0.001%. In yet another embodiment of the invention, the nanoemulsion comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion is less than about 5.0% and greater than about 0.001%.

In another embodiment of the invention, the nanoemulsion comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.05% to about 7.0%, or the non-ionic surfactant is present in a concentration of about 0.5% to about 4%. In yet another embodiment of the invention, the nanoemulsion comprises a cationic surfactant present in a concentration of about 0.01% to about 2%, in combination with a nonionic surfactant.

5. Additional Ingredients

Additional compounds suitable for use in the nanoemulsions of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion, or the additional compounds can be added to the original mixture to be emulsified. In certain of these embodiments, one or more additional compounds are admixed into an existing nanoemulsion composition immediately prior to its use.

Suitable preservatives in the nanoemulsions of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion may further comprise at least one pH adjuster. Suitable pH adjusters in the nanoemulsion of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion can comprise a chelating agent. In one embodiment of the invention, the chelating agent is present in an amount of about 0.0005% to about 1.0%. Examples of chelating agents include, but are not limited to, phytic acid, polyphosphoric acid, citric acid, gluconic acid, acetic acid, lactic acid, ethylenediamine, ethylenediaminetetraacetic acid (EDTA), and dimercaprol, and a preferred chelating agent is ethylenediaminetetraacetic acid.

The nanoemulsion can comprise a buffering agent, such as a pharmaceutically acceptable buffering agent. Examples of buffering agents include, but are not limited to, 2-Amino-2-methyl-1,3-propanediol, ≥99.5% (NT), 2-Amino-2-methyl-1-propanol, ≥99.0% (GC), L-(+)-Tartaric acid, ≥99.5% (T), ACES, ≥99.5% (T), ADA, ≥99.0% (T), Acetic acid, ≥99.5% (GC/T), Acetic acid, for luminescence, ≥99.5% (GC/T), Ammonium acetate solution, for molecular biology, ˜5 M in H₂O, Ammonium acetate, for luminescence, ≥99.0% (calc. on dry substance, T), Ammonium bicarbonate, ≥99.5% (T), Ammonium citrate dibasic, ≥99.0% (T), Ammonium formate solution, 10 M in H₂O, Ammonium formate, ≥99.0% (calc. based on dry substance, NT), Ammonium oxalate monohydrate, ≥99.5% (RT), Ammonium phosphate dibasic solution, 2.5 M in H₂O, Ammonium phosphate dibasic, ≥99.0% (T), Ammonium phosphate monobasic solution, 2.5 M in H₂O, Ammonium phosphate monobasic, ≥99.5% (T), Ammonium sodium phosphate dibasic tetrahydrate, ≥99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≥99.5% (T), BES buffered saline, for molecular biology, 2× concentrate, BES, ≥99.5% (T), BES, for molecular biology, ≥99.5% (T), BICINE buffer Solution, for molecular biology, 1 M in H₂O, BICINE, ≥99.5% (T), BIS-TRIS, ≥99.0% (NT), Bicarbonate buffer solution, ≥0.1 M Na₂CO₃, ≥0.2 M NaHCO₃, Boric acid, ≥99.5% (T), Boric acid, for molecular biology, ≥99.5% (T), CAPS, ≥99.0% (TLC), CHES, ≥99.5% (T), Calcium acetate hydrate, ≥99.0% (calc. on dried material, KT), Calcium carbonate, precipitated, ≥99.0% (KT), Calcium citrate tribasic tetrahydrate, ≥98.0% (calc. on dry substance, KT), Citrate Concentrated Solution, for molecular biology, 1 M in H₂O, Citric acid, anhydrous, ≥99.5% (T), Citric acid, for luminescence, anhydrous, ≥99.5% (T), Diethanolamine, ≥99.5% (GC), EPPS, ≥99.0% (T), Ethylenediaminetetraacetic acid disodium salt dihydrate, for molecular biology, ≥99.0% (T), Formic acid solution, 1.0 M in H₂O, Gly-Gly-Gly, ≥99.0% (NT), Gly-Gly, ≥99.5% (NT), Glycine, ≥99.0% (NT), Glycine, for luminescence, ≥99.0% (NT), Glycine, for molecular biology, ≥99.0% (NT), HEPES buffered saline, for molecular biology, 2× concentrate, HEPES, ≥99.5% (T), HEPES, for molecular biology, ≥99.5% (T), Imidazole buffer Solution, 1 M in H₂O, Imidazole, ≥99.5% (GC), Imidazole, for luminescence, ≥99.5% (GC), Imidazole, for molecular biology, ≥99.5% (GC), Lipoprotein Refolding Buffer, Lithium acetate dihydrate, ≥99.0% (NT), Lithium citrate tribasic tetrahydrate, ≥99.5% (NT), MES hydrate, ≥99.5% (T), MES monohydrate, for luminescence, ≥99.5% (T), MES solution, for molecular biology, 0.5 M in H₂O, MOPS, ≥99.5% (T), MOPS, for luminescence, ≥99.5% (T), MOPS, for molecular biology, ≥99.5% (T), Magnesium acetate solution, for molecular biology, ˜1 M in H₂O, Magnesium acetate tetrahydrate, ≥99.0% (KT), Magnesium citrate tribasic nonahydrate, ≥98.0% (calc. based on dry substance, KT), Magnesium formate solution, 0.5 M in H₂O, Magnesium phosphate dibasic trihydrate, ≥98.0% (KT), Neutralization solution for the in-situ hybridization for in-situ hybridization, for molecular biology, Oxalic acid dihydrate, ≥99.5% (RT), PIPES, ≥99.5% (T), PIPES, for molecular biology, ≥99.5% (T), Phosphate buffered saline, solution (autoclaved), Phosphate buffered saline, washing buffer for peroxidase conjugates in Western Blotting, 10× concentrate, Piperazine, anhydrous, ≥99.0% (T), Potassium D-tartrate monobasic, ≥99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≥99.0% (NT), Potassium acetate, for luminescence, ≥99.0% (NT), Potassium acetate, for molecular biology, ≥99.0% (NT), Potassium bicarbonate, ≥99.5% (T), Potassium carbonate, anhydrous, ≥99.0% (T), Potassium chloride, ≥99.5% (AT), Potassium citrate monobasic, ≥99.0% (dried material, NT), Potassium citrate tribasic solution, 1 M in H₂O, Potassium formate solution, 14 M in H₂O, Potassium formate, ≥99.5% (NT), Potassium oxalate monohydrate, ≥99.0% (RT), Potassium phosphate dibasic, anhydrous, ≥99.0% (T), Potassium phosphate dibasic, for luminescence, anhydrous, ≥99.0% (T), Potassium phosphate dibasic, for molecular biology, anhydrous, ≥99.0% (T), Potassium phosphate monobasic, anhydrous, ≥99.5% (T), Potassium phosphate monobasic, for molecular biology, anhydrous, ≥99.5% (T), Potassium phosphate tribasic monohydrate, ≥95% (T), Potassium phthalate monobasic, ≥99.5% (T), Potassium sodium tartrate solution, 1.5 M in H₂O, Potassium sodium tartrate tetrahydrate, ≥99.5% (NT), Potassium tetraborate tetrahydrate, ≥99.0% (T), Potassium tetraoxalate dihydrate, ≥99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≥99.5% (NT), Sodium acetate solution, for molecular biology, ˜3 M in H₂O, Sodium acetate trihydrate, ≥99.5% (NT), Sodium acetate, anhydrous, ≥99.0% (NT), Sodium acetate, for luminescence, anhydrous, ≥99.0% (NT), Sodium acetate, for molecular biology, anhydrous, ≥99.0% (NT), Sodium bicarbonate, ≥99.5% (T), Sodium bitartrate monohydrate, ≥99.0% (T), Sodium carbonate decahydrate, ≥99.5% (T), Sodium carbonate, anhydrous, ≥99.5% (calc. on dry substance, T), Sodium citrate monobasic, anhydrous, ≥99.5% (T), Sodium citrate tribasic dihydrate, ≥99.0% (NT), Sodium citrate tribasic dihydrate, for luminescence, ≥99.0% (NT), Sodium citrate tribasic dihydrate, for molecular biology, ≥99.5% (NT), Sodium formate solution, 8 M in H₂O, Sodium oxalate, ≥99.5% (RT), Sodium phosphate dibasic dihydrate, ≥99.0% (T), Sodium phosphate dibasic dihydrate, for luminescence, ≥99.0% (T), Sodium phosphate dibasic dihydrate, for molecular biology, ≥99.0% (T), Sodium phosphate dibasic dodecahydrate, ≥99.0% (T), Sodium phosphate dibasic solution, 0.5 M in H₂O, Sodium phosphate dibasic, anhydrous, ≥99.5% (T), Sodium phosphate dibasic, for molecular biology, ≥99.5% (T), Sodium phosphate monobasic dihydrate, ≥99.0% (T), Sodium phosphate monobasic dihydrate, for molecular biology, ≥99.0% (T), Sodium phosphate monobasic monohydrate, for molecular biology, ≥99.5% (T), Sodium phosphate monobasic solution, 5 M in H₂O, Sodium pyrophosphate dibasic, ≥99.0% (T), Sodium pyrophosphate tetrabasic decahydrate, ≥99.5% (T), Sodium tartrate dibasic dihydrate, ≥99.0% (NT), Sodium tartrate dibasic solution, 1.5 M in H₂O (colorless solution at 20° C.), Sodium tetraborate decahydrate, ≥99.5% (T), TAPS, ≥99.5% (T), TES, ≥99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate-EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≥99.5% (NT), Triethanolamine, ≥99.5% (GC), Triethylamine, ≥99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≥99.0% (NT), Trizma® base, ≥99.8% (T), Trizma® base, ≥99.8% (T), Trizma® base, for luminescence, ≥99.8% (T), Trizma® base, for molecular biology, ≥99.8% (T), Trizma® carbonate, ≥98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≥99.0% (AT), Trizma® hydrochloride, for luminescence, ≥99.0% (AT), Trizma® hydrochloride, for molecular biology, ≥99.0% (AT), and Trizma® maleate, ≥99.5% (NT).

The nanoemulsion can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsions that may readily be diluted with water to a desired concentration without impairing their antiviral properties.

6. Active Agents Incorporated into a Nanoemulsion of the Invention

In a further embodiment of the invention, a nanoemulsion comprises an additional active agent, such as an antibiotic or a palliative agent (such as for burn wound treatment). Addition of another agent may enhance the therapeutic effectiveness of the nanoemulsion. The nanoemulsion in and of itself has anti-bacterial activity and does not need to be combined with another active agent to obtain therapeutic effectiveness. Any antibacterial (or antibiotic) agent suitable for treating a bacterial infection can be incorporated into the topical nanoemulsions of the invention.

Examples of such antibiotic agents include, but are not limited to, aminoglycosides, Ansamycins, Carbacephems, Carbapenems, Cephalosporins, Glycopeptides, Macrolides, Monobactams, Penicillins, Polypeptides, Polymyxin, Quinolones, Sulfonamides, Tetracyclines, and others (e.g., Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in US), Thiamphenicol, Tinidazole, Dapsone, and lofazimine).

Examples of these classes of antibiotics include, but are not limited to, Amikacin, Gentamicin, Kanamycin, Neomycin, Netilmicin, Streptomycin, Tobramycin, Paromomycin, Geldanamycin, Herbimycin, Loracarbef, Ertapenem, Doripenem, Imipenem/Cilastatin, Meropenem, Cefadroxil, Cefazolin, Cefalotin or Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftobiprole, Teicoplanin, Vancomycin, Azithromycin, Clarithromycin, Dirithromycin, Erythromycin, Roxithromycin, Troleandomycin, Telithromycin, Spectinomycin, Aztreonam, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Bacitracin, Colistin, Polymyxin B, Ciprofloxacin, Enoxacin, Gatifloxacin, Levofloxacin, Lomefloxacin, Moxifloxacin, Norfloxacin, Ofloxacin, Trovafloxacin, Grepafloxacin, Sparfloxacin, Temafloxacin, Mafenide, Sulfonamidochrysoidine (archaic), Sulfacetamide, Sulfadiazine, Sulfamethizole, Sulfanilimide (archaic), Sulfasalazine, Sulfisoxazole, Trimethoprim, rimethoprim-Sulfamethoxazole (Co-trimoxazole) (TMP-SMX), Demeclocycline, Doxycycline, Minocycline, Oxytetracycline, and Tetracycline.

Examples of palliative agents which may be incorporated into the nanoemulsions of the invention include, but are not limited to, menthol, camphor, phenol, allantoin, benzocaine, corticosteroids, phenol, zinc oxide, camphor, pramoxine, dimethicone, meradimate, octinoxate, octisalate, oxybenzone, dyclonine, alcohols (e.g., benzyl alcohol), mineral oil, propylene glycol, titanium dioxide, silver nitrate (AgNO₃), silver sulfadiazine, mafenide acetate, nanocrystalline impregnated silver dressings, a p38 MAPK inhibitor, and magnesium stearate.

Pharmaceutical Compositions

The nanoemulsion formulations the invention may be formulated into pharmaceutical compositions that comprise the nanoemulsion in a therapeutically effective amount and suitable, pharmaceutically-acceptable excipients for administration to a human subject in need thereof using any conventional pharmaceutical method of administration. Such excipients are well known in the art.

By the phrase “therapeutically effective amount” it is meant any amount of the nanoemulsion that is effective in treating a burn wound (e.g., to prevent the progression of a partial thickness burn wound to a deep partial thickness burn wound and/or a full thickness burn wound). In some embodiments, a therapeutically effective amount is a dilution of a concentrated nanoemulsion formulation (e.g., dilutions of a concentrated composition may be administered to a subject such that the subject receives any one or more of the specific dosages provided herein). In some embodiments, dilution of a concentrated composition may be made such that a subject is administered (e.g., to a burn wound in a single or multiple doses) a composition comprising 0.5-50% of the nanoemulsion present in the concentrated composition.

Exemplary dosage forms may include, but are not limited to, patches, ointments, creams, emulsions, liquids, lotions, gels, bioadhesive gels, aerosols, pastes, foams, sunscreens, capsules, microcapsules, or in the form of an article or carrier, such as a bandage, insert, syringe-like applicator, pessary, powder, and talc or other solid.

The pharmaceutical compositions may be formulated for immediate release, sustained release, controlled release, delayed release, or any combinations thereof. In some embodiments, the formulations may comprise a penetration-enhancing agent for enhancing penetration of the nanoemulsion through the stratum corneum and into the epidermis or dermis (e.g., for methods of treating burn wounds). Suitable penetration-enhancing agents include, but are not limited to, alcohols such as ethanol, triglycerides and aloe compositions. The amount of the penetration-enhancing agent may comprise from about 0.5% to about 40% by weight of the formulation.

When appropriate, for example when treating burn wounds, the nanoemulsions of the invention can be applied and/or delivered utilizing electrophoretic delivery/electrophoresis. Such transdermal methods, which comprise applying an electrical current, are well known in the art.

In another embodiment of the invention, minimal systemic absorption of the nanoemulsion occurs upon topical administration. Such minimal systemic exposure can be determined by the detection of less than 10 ng/mL, less than 8 ng/mL, less than 5 ng/mL, less than 4 ng/mL, less than 3 ng/mL, or less than 2 ng/mL of the one or more surfactants present in the nanoemulsion in the plasma of the subject. Lack of systemic absorption may be monitored, for example, by measuring the amount of the surfactant, such as the cationic surfactant, in the plasma of the human subject undergoing treatment. Amounts of surfactant of equal to or less than about 10 ng/ml in the plasma confirms minimal systemic absorption.

The pharmaceutical compositions may be applied in a single administration or in multiple administrations. The pharmaceutical compositions can be applied for at least one day, at least two days at least three days at least four days at least 5 days, once a week, at least twice a week, at least once a day, at least twice a day, multiple times daily, multiple times weekly, biweekly, at least once a month, or any combination thereof.

Following administration, the nanoemulsion may be occluded or semi-occluded. Occlusion or semi-occlusion may be performed by overlaying a bandage, polyoleofin film, article of clothing, impermeabile barrier, or semi-impermeable barrier to the topical preparation.

Several exemplary nanoemulsions are described in Examples 1-5, although the compositions and methods of the invention are not limited to these specific nanoemulsions. For example, the invention provides a nanoemulsion composition identified utilizing compositions and described herein (e.g., compositions and methods for identifying and characterizing nanoemulsions useful for the treatment of burn wounds (See, e.g., Examples 1-5)). The components and quantity of each can be varied as described herein in the preparation of other nanoemulsions.

Exemplary emulsions of the invention are provided in the Examples (e.g., in Example 1, Tables 10 and 11, below).

In some embodiments, nanoemulsion formulations of the invention have an average particle (droplet) size of about 200 nm to about 600 nm. In more preferred embodiments, nanoemulsion formulations of the invention have an average particle (droplet) size of about 300 nm-400 nm, 325 nm-375 nm, 350 nm-370 nm, 360 nm, although smaller (e.g., less than about 300 nm) and larger (e.g., greater than 400 nm) particle sizes also find use in the compositions and methods described herein). In a preferred embodiments, nanoemulsion formulations of the invention undergoes high pressure processing in order to have a particle (droplet) size of about 360 nm.

Methods of Manufacture

Nanoemulsion formulations of the invention can be formed using classic emulsion forming techniques (See, e.g., U.S. 2004/0043041. See also U.S. Pat. Nos. 6,015,832, 6,506,803, 6,559,189, 6,635,676, and US Patent Publication No. 20040043041, all of which are incorporated by reference). In addition, methods of making emulsions are described in U.S. Pat. Nos. 5,103,497 and 4,895,452 (herein incorporated by reference). In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water. The nanoemulsions of the invention are stable, and do not decompose even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanoemulsion can have textures ranging from that of a semi-solid cream to that of a thin lotion, and can be applied topically by hand, and can be sprayed onto a surface or nebulized.

As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsions of the invention should kill or disable microorganisms in vitro. To determine the suitability of a particular candidate nanoemulsion against a desired microorganism, the nanoemulsion is exposed to the microorganism for one or more time periods in a side-by-side experiment with an appropriate control sample (e.g., a negative control such as water) and determining if, and to what degree, the nanoemulsion kills or disables the microorganism.

The nanoemulsion of the invention can be provided in many different types of containers and delivery systems. For example, in some embodiments of the invention, the nanoemulsions are provided as a liquid, lotion, cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles (e.g., pressurized spray bottles, nebulizers).

Exemplary Methods of Use

As described in more detail throughout this application, the present invention is directed to methods of treating burn wounds. In general, the method comprises administering a nanoemulsion to a burn wound harbored by a subject, wherein the nanoemulsion comprises: (i) water; (ii) at least one organic solvent; (iii) at least one surfactant; and (iv) at least one oil; and wherein the nanoemulsion comprises droplets having an average diameter of less than about 1000 nm. The nanoemulsion can be delivered using any pharmaceutically acceptable means.

In yet another embodiment, the invention is directed to a method of treating a burn wound and/or preventing burn wound progression/conversion in a subject having a burn wound, wherein: (a) the method comprises administering a nanoemulsion to the subject; and (b) the nanoemulsion comprises: (i) water; (ii) at least one organic solvent; (iii) at least one surfactant; and (iv) at least one oil; and wherein the nanoemulsion comprises droplets having an average diameter of less than about 1000 nm. In one embodiment of the invention, the subject is susceptible to or has an infection by one or more gram-negative or gram-positive bacterial species. In another embodiment, the bacterial species are selected from the group consisting of Staphylococcus spp., Haemophilus spp., Pseudomonas spp., Burkholderia spp., Acinetobacter spp, Stenotrophomonas spp., Escherichia spp., Klebsiella spp., and Proteus spp. The nanoemulsion can be delivered using any pharmaceutically acceptable means, with inhalation, nebulization, and topical application to mucosal surfaces being examples of useful administration methods.

In yet another embodiment, the invention is directed to a method of treating or preventing an Haemophilus influenzae infection in a subject wherein: (a) the method comprises administering a nanoemulsion to the subject having or at risk of having a Haemophilus influenzae infection; (b) the nanoemulsion comprises: (i) water; (ii) at least one organic solvent; (iii) at least one surfactant; and (iv) at least one oil; and (c) wherein the nanoemulsion comprises droplets having an average diameter of less than about 1000 nm. The nanoemulsion can be delivered using any pharmaceutically acceptable means.

In one embodiment of the invention, the nanoemulsion exhibits minimal or no toxicity or side effects. Preferably, the nanoemulsion does not exhibit resistance to bacteria.

Methods described herein may further comprise administering one or more antibiotics either before, during, or after administration of the nanoemulsion. In yet another embodiment, one or more antibiotics may be incorporated into a nanoemulsion. In yet another embodiment of the invention, the nanoemulsion does not exhibit any antagonism with the antibiotic.

In one embodiment of the invention, administration of a nanoemulsion and at least one antibiotic is synergistic as defined by a fractional inhibitory concentration (FIC) index, a fractional bactericidal concentration (FBC) index, or a combination thereof. This embodiment applies to all methods described herein. Examples of such antibiotics include, but are not limited to polymyxins (colistin) and aminoglycosides (tobramycin).

In yet another embodiment, the methods of the invention may be used to treat or prevent infection by one or more bacterial species selected from the group consisting of Pseudomonas aeruginosa, B. cenocepacia, A. baumannii, Stenotrophomonas maltophilia, Staphylococcus aureus, H influenzae, E. coli, K pneumoniae, and Proteus mirabilis. All other gram positive or gram negative bacteria are also encompassed by the methods of the invention.

In one embodiment, the minimum inhibitory concentration (MIC), the minimum bactericidal concentration (MBC), or a combination thereof for the nanoemulsion demonstrate bacteriostatic or bactericidal activity for the nanoemulsion. This embodiment applies to all methods described herein.

In another embodiment of the invention, one or more bacterial species may exhibit resistance against one or more antibiotics. For example, the bacterial species can be methicillin-resistant Staphylococcus aureus (MRSA). This embodiment applies to all methods described herein.

The present invention is not limited by the type of subject administered a composition of the present invention. Each subject (e.g., harboring a burn wound) described herein may be administered a composition of the present invention.

The present invention is not limited by the particular formulation of a composition comprising a nanoemulsion of the present invention. Indeed, a composition comprising a nanoemulsion of the present invention may comprise one or more different agents in addition to the nanoemulsion. These agents or cofactors include, but are not limited to, adjuvants, surfactants, additives, buffers, solubilizers, chelators, oils, salts, therapeutic agents, drugs, bioactive agents, antibacterials, and antimicrobial agents (e.g., antibiotics, antivirals, etc.). In some embodiments, a composition comprising a nanoemulsion of the present invention comprises an agent and/or co-factor that enhance the ability of the nanoemulsion to prevent progression/conversion of a burn wound, inhibit pain and/or to kill a microbe. In some preferred embodiments, the presence of one or more co-factors or agents reduces the amount of nanoemulsion required for a desired effect. The present invention is not limited by the type of co-factor or agent used in a therapeutic agent of the present invention.

In some embodiments, a co-factor or agent used in a nanoemulsion composition is a bioactive agent. For example, in some embodiments, the bioactive agent may be a bioactive agent useful in a cell (e.g., a cell expressing a CFTR). Bioactive agents, as used herein, include diagnostic agents such as radioactive labels and fluorescent labels. Bioactive agents also include molecules affecting the metabolism of a cell (e.g., a cell expressing a CFTR), including peptides, nucleic acids, and other natural and synthetic drug molecules. Bioactive agents include, but are not limited to, adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunct to; anesthetic; anorectic; antagonist; anterior pituitary suppressant; anthelmintic; anti-acne agent (e.g., tetramethylhexadecenyl succinyl cysteine, Adapalene, Adapalene/benzoyl peroxide, Azelaic acid, Benzamycin, Benzoyl peroxide, Benzoyl peroxide/clindamycin, clindamycin, clindamycin/tretinoin, dapsone, doxycycline, epristeride, erythromycin/isotretinoin, glycolic acid, isotretinoin, lymecycline, mesulfen, metogest, minocycline, motretinide, salicylic acid, MT D002, STRIDEX, Sulfacetamide, sulfacetamide/sulfur, sulfur, tazarotene, tetracycline, tioxolone, tretinoin); anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; antidote; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; inhibitor; keratolytic; LHRH agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; pulmonary surface; radioactive agent; regulator; relaxant; repartitioning agent; scabicide; sclerosing agent; sedative; sedative-hypnotic; selective adenosine A1 antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; amyotrophic lateral sclerosis agent; cerebral ischemia agent; Paget's disease agent; unstable angina agent; uricosuric; vasoconstrictor; vasodilator; vulnerary; wound healing agent; xanthine oxidase inhibitor.

Molecules useful as antimicrobials can be delivered by the methods and compositions of the invention. Antibiotics that may find use in co-administration with a composition comprising a nanoemulsion of the present invention include, but are not limited to, agents or drugs that are bactericidal and/or bacteriostatic (e.g., inhibiting replication of bacteria or inhibiting synthesis of bacterial components required for survival of the infecting organism), including, but not limited to, almecillin, amdinocillin, amikacin, amoxicillin, amphomycin, amphotericin B, ampicillin, azacitidine, azaserine, azithromycin, azlocillin, aztreonam, bacampicillin, bacitracin, benzyl penicilloyl-polylysine, bleomycin, candicidin, capreomycin, carbenicillin, cefaclor, cefadroxil, cefamandole, cefazoline, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezolid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, Pep5, RP 59500, and TD-6424.

In some embodiments, a composition comprising a nanoemulsion of the present invention comprises one or more mucoadhesives (See, e.g., U.S. Pat. App. No. 20050281843, hereby incorporated by reference in its entirety). The present invention is not limited by the type of mucoadhesive utilized. Indeed, a variety of mucoadhesives are contemplated to be useful in the present invention including, but not limited to, cross-linked derivatives of poly(acrylic acid) (e.g., carbopol and polycarbophil), polyvinyl alcohol, polyvinyl pyrollidone, polysaccharides (e.g., alginate and chitosan), hydroxypropyl methylcellulose, lectins, fimbrial proteins, and carboxymethylcellulose.

In some embodiments, a composition of the present invention may comprise sterile aqueous preparations. Acceptable vehicles and solvents include, but are not limited to, water, Ringer's solution, phosphate buffered saline and isotonic sodium chloride solution. In addition, sterile, fixed oils are conventionally employed as a solvent or suspending medium. For this purpose any bland fixed mineral or non-mineral oil may be employed including synthetic mono-ordi-glycerides. In addition, fatty acids such as oleic acid find use in the preparation of injectables. Carrier formulations suitable for mucosal, pulmonary, subcutaneous, intramuscular, intraperitoneal, intravenous, or administration via other routes may be found in Remington's Pharmaceutical Sciences, Mack Publishing Company, Easton, Pa.

A composition comprising a nanoemulsion of the present invention can be used therapeutically or as a prophylactic. A composition comprising a nanoemulsion of the present invention can be administered to a subject via a number of different delivery routes and methods.

For example, the compositions of the present invention can be administered to a subject by multiple methods, including, but not limited to: being suspended in a solution and applied to a surface; being suspended in a solution and sprayed onto a surface using a spray applicator; being mixed with a mucoadhesive and applied (e.g., sprayed or wiped) onto a surface (e.g., mucosal or pulmonary surface); being placed on or impregnated onto a nasal and/or pulmonary applicator and applied; being applied by a controlled-release mechanism; applied using a nebulizer, aerosolized, being applied as a liposome; or being applied on a polymer. The present invention is not limited by the route of administration.

Topical formulations may be presented as, for instance, ointments, creams or lotions, foams, and aerosols, and may contain appropriate conventional additives such as preservatives, solvents (e.g., to assist penetration), and emollients in ointments and creams.

Topical formulations may also include agents that enhance penetration of the active ingredients through the skin. Exemplary agents include a binary combination of N-(hydroxyethyl) pyrrolidone and a cell-envelope disordering compound, a sugar ester in combination with a sulfoxide or phosphine oxide, and sucrose monooleate, decyl methyl sulfoxide, and alcohol.

Other exemplary materials that increase skin penetration include surfactants or wetting agents including, but not limited to, polyoxyethylene sorbitan mono-oleoate (Polysorbate 80); sorbitan mono-oleate (Span 80); p-isooctyl polyoxyethylene-phenol polymer (Triton WR-1330); polyoxyethylene sorbitan tri-oleate (Tween 85); dioctyl sodium sulfosuccinate; and sodium sarcosinate (Sarcosyl NL-97); and other pharmaceutically acceptable surfactants.

In certain embodiments of the invention, compositions may further comprise one or more alcohols, zinc-containing compounds, emollients, humectants, thickening and/or gelling agents, neutralizing agents, and surfactants. Water used in the formulations is preferably deionized water having a neutral pH. Additional additives in the topical formulations include, but are not limited to, silicone fluids, dyes, fragrances, pH adjusters, and vitamins.

Topical formulations may also contain compatible conventional carriers, such as cream or ointment bases and ethanol or oleyl alcohol for lotions. Such carriers may be present as from about 1% up to about 98% of the formulation. The ointment base can comprise one or more of petrolatum, mineral oil, ceresin, lanolin alcohol, panthenol, glycerin, bisabolol, cocoa butter and the like.

Methods of intranasal and pulmonary administration are well known in the art, including the administration of a droplet or spray form of the nanoemulsion into the nasopharynx of a subject to be treated. In some embodiments, a nebulized or aerosolized composition comprising a nanoemulsion is provided. Enteric formulations such as gastro resistant capsules for oral administration, suppositories for rectal or vaginal administration may also form part of this invention. Compositions of the present invention may also be administered via the oral route. Under these circumstances, a composition comprising a nanoemulsion may comprise a pharmaceutically acceptable excipient and/or include alkaline buffers, or enteric capsules. Formulations for nasal delivery may include those with dextran or cyclodextran and saponin as an adjuvant.

In some embodiments, an aqueous solution containing the nanoemulsion is gently and thoroughly mixed to form a solution. The solution is sterile filtered (e.g., through a 0.2 micron filter) into a sterile, enclosed vessel. Under sterile conditions, the solution is passed through an appropriately small orifice to make droplets (e.g., between 0.1 and 10 microns).

The particles may be administered using any of a number of different applicators. Suitable methods for manufacture and administration are described in the following U.S. Pat. Nos. 6,592,904; 6,518,239; 6,423,344; 6,294,204; 6,051,256 and 5,997,848 to INHALE (now NEKTAR); and U.S. Pat. No. 5,985,309; RE37,053; U.S. Pat. Nos. 6,436,443; 6,447,753; 6,503,480; and U.S. Pat. No. 6,635,283, to Edwards, et al. (MIT, AIR), each of which is hereby incorporated

Thus, in some embodiments, compositions of the present invention are administered by pulmonary delivery. For example, a composition of the present invention can be delivered to the lungs of a subject (e.g., a human) via inhalation (See, e.g., Adjei, et al. Pharmaceutical Research 1990; 7:565-569; Adjei, et al. Int. J. Pharmaceutics 1990; 63:135-144; Braquet, et al. J. Cardiovascular Pharmacology 1989 143-146; Hubbard, et al. (1989) Annals of Internal Medicine, Vol. III, pp. 206-212; Smith, et al. J. Clin. Invest. 1989; 84:1145-1146; Oswein, et al. “Aerosolization of Proteins”, 1990; Proceedings of Symposium on Respiratory Drug Delivery II Keystone, Colo.; Debs, et al. J. Immunol. 1988; 140:3482-3488; and U.S. Pat. No. 5,284,656 to Platz, et al, each of which are hereby incorporated by reference in its entirety). A method and composition for pulmonary delivery of drugs for systemic effect is described in U.S. Pat. No. 5,451,569 to Wong, et al., hereby incorporated by reference; See also U.S. Pat. No. 6,651,655 to Licalsi et al., hereby incorporated by reference in its entirety)). In some embodiments, a composition comprising a nanoemulsion is administered to a subject by more than one route or means (e.g., administered via pulmonary route as well as a mucosal route).

Further contemplated for use in the practice of this invention are a wide range of mechanical devices designed for pulmonary and/or nasal mucosal delivery of pharmaceutical agents including, but not limited to, nebulizers, metered dose inhalers, and powder inhalers, all of which are familiar to those skilled in the art. Some specific examples of commercially available devices suitable for the practice of this invention are the ULTRAVENT nebulizer (Mallinckrodt Inc., St. Louis, Mo.); the ACORN II nebulizer (Marquest Medical Products, Englewood, Colo.); the VENTOLIN metered dose inhaler (Glaxo Inc., Research Triangle Park, N.C.); and the SPINHALER powder inhaler (Fisons Corp., Bedford, Mass.). All such devices require the use of formulations suitable for dispensing of the therapeutic agent. Typically, each formulation is specific to the type of device employed and may involve the use of an appropriate propellant material, in addition to the usual diluents, adjuvants, surfactants, carriers and/or other agents useful in therapy. Also, the use of liposomes, microcapsules or microspheres, inclusion complexes, or other types of carriers is contemplated.

As described above, the present invention is not limited by the type of subject administered a composition of the present invention. Indeed, a wide variety of subjects are contemplated to be benefited from administration of a composition of the present invention. In preferred embodiments, the subject is a human. In some embodiments, human subjects are of any age (e.g., adults, children, infants, etc.) that have a burn wound. In some embodiments, the human subjects are subjects that are more likely to receive a direct exposure to pathogenic microorganisms or that are more likely to display signs and symptoms of disease after exposure to a pathogen (e.g., subjects in the armed forces, government employees, frequent travelers, persons attending or working in a school or daycare, health care workers, an elderly person, an immunocompromised person, and emergency service employees (e.g., police, fire, EMT employees)). In some embodiments, any one or all members of the general public can be administered a composition of the present invention.

A composition comprising a nanoemulsion of the present invention can be administered (e.g., to a subject or to microbes (e.g., bacteria (e.g., opportunistic and/or pathogenic bacteria (e.g., residing on or within a burn wound)))) as a therapeutic or as a prophylactic to prevent microbial infection. Thus, in some embodiments, the present invention provides a method of altering microbial (e.g., bacterial (e.g., opportunistic and/or pathogenic bacterial) growth comprising administering a composition comprising a nanoemulsion to the microbes (e.g., bacteria (e.g., opportunistic and/or pathogenic bacteria). In some embodiments, administration of a composition comprising a nanoemulsion to the microbes (e.g., bacteria (e.g., opportunistic and/or pathogenic bacteria) kills the microbes. In some embodiments, administration of a composition comprising nanoemulsion to the microbes (e.g., bacteria (e.g., opportunistic and/or pathogenic bacteria) inhibits growth of the microbes. It is contemplated that a composition comprising a nanoemulsion can be administered to microbes (e.g., bacteria (e.g., opportunistic and/or pathogenic bacteria (e.g., residing within the respiratory tract))) via a number of delivery routes and/or mechanisms.

Compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the components of the compositions of the present invention. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the nanoemulsion. In some embodiments, nanoemulsion compositions of the present invention are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

In some embodiments, a composition comprising a nanoemulsion is co-administered with one or more antibiotics. For example, one or more antibiotics may be administered with, before and/or after administration of a composition comprising a nanoemulsion. The present invention is not limited by the type of antibiotic co-administered. Indeed, a variety of antibiotics may be co-administered including, but not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, doxycycline, quinolones (e.g., ciprofloxacin), sulfonamides, trimethoprim, and quinolines.

A wide variety of antimicrobial agents are currently available for use in treating bacterial, fungal and viral infections. For a comprehensive treatise on the general classes of such drugs and their mechanisms of action, the skilled artisan is referred to Goodman & Gilman's “The Pharmacological Basis of Therapeutics” Eds. Hardman et al., 9th Edition, Pub. McGraw Hill, chapters 43 through 50, 1996, (herein incorporated by reference in its entirety). Generally, these agents include agents that inhibit cell wall synthesis (e.g., penicillins, cephalosporins, cycloserine, vancomycin, bacitracin); and the imidazole antifungal agents (e.g., miconazole, ketoconazole and clotrimazole); agents that act directly to disrupt the cell membrane of the microorganism (e.g., detergents such as polmyxin and colistimethate and the antifungals nystatin and amphotericin B); agents that affect the ribosomal subunits to inhibit protein synthesis (e.g., chloramphenicol, the tetracyclines, erthromycin and clindamycin); agents that alter protein synthesis and lead to cell death (e.g., aminoglycosides); agents that affect nucleic acid metabolism (e.g., the rifamycins and the quinolones); the antimetabolites (e.g., trimethoprim and sulfonamides); and the nucleic acid analogues such as zidovudine, gangcyclovir, vidarabine, and acyclovir which act to inhibit viral enzymes essential for DNA synthesis. Various combinations of antimicrobials may be employed.

The present invention also includes methods involving co-administration of a composition comprising a nanoemulsion with one or more additional active and/or anti-infective agents. In co-administration procedures, the agents may be administered concurrently or sequentially. In one embodiment, the compositions described herein are administered prior to the other active agent(s). The pharmaceutical formulations and modes of administration may be any of those described herein. In addition, the two or more co-administered agents may each be administered using different modes (e.g., routes) or different formulations. The additional agents to be co-administered (e.g., antibiotics, a second type of nanoemulsion, etc.) can be any of the well-known agents in the art, including, but not limited to, those that are currently in clinical use.

In some embodiments, a composition comprising a nanoemulsion is administered to a subject via more than one route. For example, a subject may benefit from receiving mucosal administration (e.g., nasal administration or other mucosal routes described herein) and, additionally, receiving one or more other routes of administration (e.g., pulmonary administration (e.g., via a nebulizer, inhaler, or other methods described herein.

Other delivery systems can include time-release, delayed release or sustained release delivery systems. Such systems can avoid repeated administrations of the compositions, increasing convenience to the subject and a physician. Many types of release delivery systems are available and known to those of ordinary skill in the art. They include polymer based systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, hereby incorporated by reference. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, each of which is hereby incorporated by reference and (b) diffusional systems in which an active component permeates at a controlled rate from a polymer such as described in U.S. Pat. Nos. 3,854,480, 5,133,974 and 5,407,686, each of which is hereby incorporated by reference. In addition, pump-based hardware delivery systems can be used, some of which are adapted for implantation.

The present invention is not limited by the amount of nanoemulsion used. In some preferred embodiments, the amount of nanoemulsion in a composition comprising a nanoemulsion is selected as that amount which treats a burn wound (e.g., prevents conversion of a partial thickness burn wound to a deep partial thickness burn wound and/or a full thickness burn wound) without significant, adverse side effects. The amount will vary depending upon which specific nanoemulsion(s) is/are employed, and can vary from subject to subject, depending on a number of factors including, but not limited to, the species, age and general condition (e.g., health) of the subject, and the mode of administration. Procedures for determining the appropriate amount of nanoemulsion administered to a subject can be readily determined using known means by one of ordinary skill in the art.

In some embodiments, it is expected that each dose (e.g., of a composition comprising a nanoemulsion (e.g., administered to a subject) comprises 1-100% nanoemulsion, in some embodiments, 20% nanoemulsion, in some embodiments less than 20% (e.g., 15%, 10%, 8%, 5% or less nanoemulsion), and in some embodiments greater than 20% nanoemulsion (e.g., 25%, 30%, 35%, 40%, 50%, 60%, or more nanoemulsion).

In some embodiments, it is expected that each dose (e.g., of a composition comprising a nanoemulsion (e.g., administered to a subject) is from 0.001 to 40% or more (e.g., 0.001-10%, 0.5-5%, 1-3%, 2%, 6%, 10%, 15%, 20%, 30%, 40% or more) by weight nanoemulsion.

Similarly, the present invention is not limited by the duration of time a nanoemulsion is administered to a subject. In some embodiments, a nanoemulsion is administered one or more times (e.g. twice, three times, four times or more) daily. In some embodiments, a composition comprising a nanoemulsion is administered one or more times a day. In some embodiments, a composition comprising a nanoemulsion of the present invention is formulated in a concentrated dose that is diluted prior to administration to a subject. For example, dilutions of a concentrated composition may be administered to a subject such that the subject receives any one or more of the specific dosages provided herein. In some embodiments, dilution of a concentrated composition may be made such that a subject is administered (e.g., in a single dose) a composition comprising 0.5-50% of the nanoemulsion present in the concentrated composition. Concentrated compositions are contemplated to be useful in a setting in which large numbers of subjects may be administered a composition of the present invention (e.g., a hospital). In some embodiments, a composition comprising a nanoemulsion of the present invention (e.g., a concentrated composition) is stable at room temperature for more than 1 week, in some embodiments for more than 2 weeks, in some embodiments for more than 3 weeks, in some embodiments for more than 4 weeks, in some embodiments for more than 5 weeks, and in some embodiments for more than 6 weeks.

Dosage units may be proportionately increased or decreased based on several factors including, but not limited to, the weight, age, and health status of the subject. In addition, dosage units may be increased or decreased for subsequent administrations.

In some embodiments, a composition comprising a nanoemulsion is administered to a subject under conditions such that microbes (e.g., bacteria (e.g., opportunistic and/or pathogenic bacteria)) are killed. In some embodiments, a composition comprising a nanoemulsion is administered to a subject under conditions such that microbial (e.g., bacterial (e.g., opportunistic and/or pathogenic bacterial) growth is prohibited and/or attenuated. In some embodiments, greater than 90% (e.g., greater than 95%, 98%, 99%, all detectable) of microbes (e.g., bacteria (e.g., opportunistic and/or pathogenic bacteria) are killed. In some embodiments, there is greater than 2 log (e.g., greater than 3 log, 4 log, 5 log, or more) reduction in microbe (e.g., bacteria (e.g., opportunistic and/or pathogenic bacteria) presence. In some embodiments, reduction and/or killing is observed in one hour or less (e.g., 45 minutes, 30 minutes, 15 minutes, or less). In some embodiments, reduction and/or killing is observed in 6 hours or less (e.g., 5 hours, 4, hours, 3 hours, two hours or less than one hour). In some embodiments, reduction and/or killing is observed in two days or less following initial treatment (e.g., less than 24 hours, less than 20 hours, 18 hours or less). In some embodiments, the reduction and/or killing is observed in three days or less, four days or less, or five days or less.

A composition comprising a nanoemulsion of the present invention finds use where the nature of the infectious and/or disease causing agent (e.g., causing signs, symptoms or indications of respiratory infection) is known, as well as where the nature of the infectious and/or disease causing agent is unknown (e.g., in emerging disease (e.g., of pandemic proportion (e.g., influenza or other outbreaks of disease))). For example, the present invention contemplates use of the compositions of the present invention in treatment of or prevention of infections associated with an emergent infectious and/or disease causing agent yet to be identified (e.g., isolated and/or cultured from a diseased person but without genetic, biochemical or other characterization of the infectious and/or disease causing agent).

It is contemplated that the compositions and methods of the present invention will find use in various settings, including research settings. For example, compositions and methods of the present invention also find use in studies of the immune system (e.g., characterization of adaptive immune responses (e.g., protective immune responses (e.g., mucosal or systemic immunity))). Uses of the compositions and methods provided by the present invention encompass human and non-human subjects and samples from those subjects, and also encompass research applications using these subjects. Compositions and methods of the present invention are also useful in studying and optimizing nanoemulsions and other components and for screening for new components. Thus, it is not intended that the present invention be limited to any particular subject and/or application setting.

The formulations can be tested in vivo in a number of animal models developed for the study of topical routes of delivery.

In some embodiments, the present invention provides a kit comprising a composition comprising a nanoemulsion. In some embodiments, the kit further provides a device or material for administering the composition. The present invention is not limited by the type of device or material included in the kit. In some embodiments, a kit comprises a composition comprising a nanoemulsion in a concentrated form (e.g., that can be diluted prior to administration to a subject).

In some embodiments, all kit components are present within a single container (e.g., vial or tube). In some embodiments, each kit component is located in a single container (e.g., vial or tube). In some embodiments, one or more kit components are located in a single container (e.g., vial or tube) with other components of the same kit being located in a separate container (e.g., vial or tube). In some embodiments, a kit comprises a buffer. In some embodiments, the kit further comprises instructions for use.

Nanoemulsion formulations and compositions comprising the same described herein may additionally contain other adjunct components conventionally found in pharmaceutical compositions. Thus, for example, the compositions may contain additional, compatible, pharmaceutically-active materials such as, for example, antipuritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, preferably do not unduly interfere with the biological activities of the immunogenic compositions described herein. The formulations can be sterilized and, if desired, mixed with auxiliary agents (e.g., lubricants, preservatives, stabilizers, wetting agents, emulsifiers, salts for influencing osmotic pressure, buffers, colorings, flavorings and/or aromatic substances and the like) that do not deleteriously interact with the NE and immunogen of the formulation. In some embodiments, immunogenic compositions described herein are administered in the form of a pharmaceutically acceptable salt. When used the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include, but are not limited to, acetic acid and a salt (1-2% w/v); citric acid and a salt (1-3% w/v); boric acid and a salt (0.5-2.5% w/v); and phosphoric acid and a salt (0.8-2% w/v). Suitable preservatives may include benzalkonium chloride (0.003-0.03% w/v); chlorobutanol (0.3-0.9% w/v); parabens (0.01-0.25% w/v) and thimerosal (0.004-0.02% w/v).

Generally, the emulsion compositions of the invention will comprise at least 0.001% to 100%, preferably 0.01 to 90%, of emulsion per ml of liquid composition. It is envisioned that the formulations may comprise about 0.001%, about 0.0025%, about 0.005%, about 0.0075%, about 0.01%, about 0.025%, about 0.05%, about 0.075%, about 0.1%, about 0.25%, about 0.5%, about 1.0%, about 2.5%, about 5%, about 7.5%, about 10%, about 12.5%, about 15%, about 20%, about 25%, about 30%, about 35%, about 40%, about 50%, about 55%, about 60%, about 65%, about 70%, about 75%, about 80%, about 85%, about 90%, about 95% or about 98% of emulsion per ml of liquid composition. It should be understood that a range between any two figures listed above is specifically contemplated to be encompassed within the metes and bounds of the present invention. Some variation in dosage will necessarily occur depending on the condition of the specific pathogen and the subject being immunized.

### EXPERIMENTAL

The following examples are provided in order to demonstrate and further illustrate certain preferred embodiments and aspects of the present invention and are not to be construed as limiting the scope thereof.

### Example 1

### Novel Nanoemulsion Formulations and Stability

Experiments were conducted in order to generate and characterize novel nanoemulsions. A total of 50 formulations were produced by varying 5 different cationic surfactants and/or by varying the ratio of cationic to non-ionic surfactants (surfactant blend) in the nanoemulsion (NE) formulation within a particular surfactant family. Table 1 provides a summary of the number of nanoemulsion that passed or failed. Table 2 describes the cationic surfactant used in these studies. Table 3 shows two of the nonionic surfactant used in these studies. Table 4 shows the qualitative formula for the various nanoemulsions when the surfactant blend ratio of the cationic surfactant is altered from 6:1 to 1:1 to 1:6, etc.

The oil-in-water nanoemulsion were manufactured at a 500 gram scale by combining the excipients using simple mixing followed by high shear homogenization. This mixture was homogenized for 10 minutes using a Silverson L4RT Batch Homogenizer with Fine Emulsion Screen at 10,000 rpm. All the ingredients in the nanoemulsion meet USP/NF Pharmacopoeia compendial requirements and are included in the CDER List of Inactive Ingredients for Approved Drug Products database. The concentrated product (100% NE) was diluted by simple mixing to achieve desired concentration for use (e.g., 10%, 20%, 30% or 40% NE).

The formulations were then placed on stability for 2 weeks at 22° C. and 40° C. The physical characteristics of the nanoemulsion were measured by particle size analysis and zeta potential. Dynamic light scattering using the Malvern Zetasizer was used to determine particle size, particle size distribution profiles and the polydispersity index after completion of the manufacture by diluting the 100% nanoemulsion to 1% in deionize distilled water pre-filtered through a 0.22 μm filter. The acceptance criteria for particle size was that there was an absence of change in the mean particle size greater than 30% from the original mean particle size. A change greater than 30% was considered a failure. The appearance was also monitored. The passing criterion for appearance was no phase separation. If there was phase separation of the formulations, the formulation failed.

Tables 4-8 provide details of each manufactured formulation with respect to composition and stability results. Table 1 is a summary of the total number of formulations that passed or failed stability assessment based on physical appearance (no evidence of phase separation) and uniform particle size (uni-modal distribution and change in PS within 30% of original median size) at time zero and after storage for 2 weeks under accelerated conditions of 22° C. and 40° C.

TABLE 1
Overall summary of the number of nanoemulsion formulations
manufactured and placed upon stability.

| Type of Cationic Nanoemulsions | Pass | Fail |
|---|---|---|
| Cetylpyridium chloride (CPC) | 16 | 0 |
| Benzalkonium chloride (BCL) | 5 | 4 |
| Benzethonium chloride (BEC) | 3 | 0 |
| Stearalkonium chloride (SAC) | 4 | 0 |
| Cocamidopropyl betaine (CAB) | 2 | 2 |
| Dioctadecyl dimethyl ammonium chloride (DODAC) | 11 | 3 |
| Total Manufactured | 41 | 9 |

Several of the cationic surfactants used during development of embodiments of the invention are listed in Table 2. The structures of two nonionic surfactants that were combined with the cationic surfactants during development of some of the embodiments of the invention are shown in Table 3.

TABLE 2
Summary of cationic surfactants used in stability testing.

| Name of | | | | | |
|---|---|---|---|---|---|
| Cationic | | | CMC | Chain | |
| Surfactant | MW | HLB | (mM) | Length | Structure |
| Cetylpyridiniu m Chloride (CPC) | 339 | 26 | 0.1 | 16 | 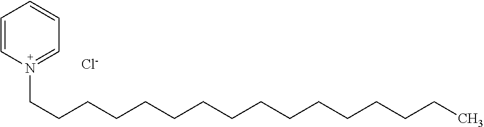 |
| Benzalkonium Chloride (BAC) | 354 | 24 | 0.47 | 6-10 | 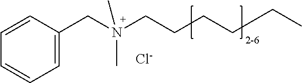 |
| Benzethonium chloride (BEC) | 448 | 15 | 0.84 | 4 | 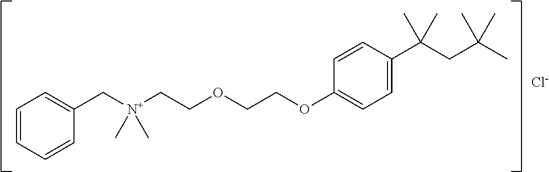 |
| Steralkonium chloride (SAC) | 424 | 11 | — | 18 | 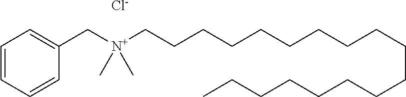 [SMILES: [Cl-] ; CCCCCCCCCCCCCCCCCC[N+](C)(C)Cc1ccccc1] |
| Cocamidopropy 1 betaine (CAB) | 342 | 11 | 0.105 | 11 | 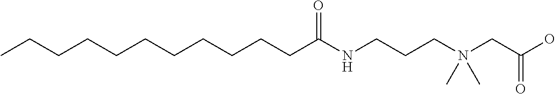 |
| Dioctadecyl dimethyl ammonium chloride (DODAC) | 586 | NA | 10* | 18 | 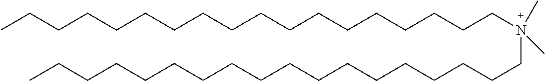 |

*Critical concentration for unilamellar vesicles

TABLE 4A
Examples of Quantitative Composition of 100% Nanoemulsion Formulations.

| | | Varying Surfactant Blend Ratios (cationic:nonionic) of 100% Nanoemulsion | | | | | |
|---|---|---|---|---|---|---|---|
| | | 6:1 | 1:1 | 1:6 | 1:10 | 1:20 | |
| Ingredients | Function: | (% w/w) | (% w/w) | (% w/w) | (% w/w) | (% w/w) | 1:40 |
| Sterile Water (USP) | Aqueous | 23.44 | 23.50 | 23.46 | 23.48 | 23.48 | 23.48 |
| | Diluent | | | | | | |
| Soybean Oil (USP) | Hydrophobic | 62.79 | 62.79 | 62.79 | 62.79 | 62.79 | 62.79 |
| (Super-refined) | oil | | | | | | |
| Dehydrated Alcohol (USP) | Organic | 6.73 | 6.73 | 6.73 | 6.73 | 6.73 | 6.73 |
| | solvent | | | | | | |
| Nonionic Surfactant | Emulsifying | 1.068 | 3.49 | 5.92 | 6.30 | 6.65 | 6.825 |
| | agent | | | | | | |
| Cationic Surfactant | Emulsifying | 6.000 | 3.49 | 1.098 | 0.7 | 0.35 | 0.175 |
| | agent, | | | | | | |
| | preservative | | | | | | |
| | and active | | | | | | |

| | | | | | | |
|---|---|---|---|---|---|---|
| Total | 100.00% | 100.00% | 100.00% | 100.00% | 100.00% | 100.00% |

Stability of Novel Nanoemulsion Formulations

Particle size is an additional property of nanoemulsions which may impact antimicrobial activity. The library of different nanoemulsions shown in Tables 4-8 provides multiple families of compounds with varying particle sizes ranging from 211-585 nm. The majority of these nanoemulsion formulations were manufactured by homogenization.

Based on the overall stability results, formulations containing CPC in combination with various nonionic surfactants at varying ratios are stable as shown in Table 4B. However, other formulations containing benzalkonium chloride (BAC) were unstable when the surfactant blend ratio (cationic:nonionic) of BAC to non-ionic surfactant changed from 1:6 to 1:1 or 6:1 (Table 5). Cationic surfactants in addition to CPC and BAC were investigated and their structure, molecular weight, HLB and CMC are summarized in Table 2 (above). Accordingly, experiments were conducted in order to investigate how the polar head unit of the cationic surfactant affects the stability of the emulsion.

TABLE 4B
Stability Results of the Cetypyridinum Chloride Nanoemulsion Formulations

| | % Cationic | % Nonionic | | | Appearance | Particle Size |
|---|---|---|---|---|---|---|
| | Surfactant | Surfactant | Surfactant | Ave Mean | (Pass/Fail at | (Pass/Fail at |
| | in Neat | in Neat | Blend Ratio | Particle | 2 wks at 22° | 2 wks at 22° |
| Series | (Positive) | (Neutral) | (Cat/Non) | Size (nm) | and 40° C.) | and 40° C.) |
| AX | CPC | Tween 80 | | | | |
| AX1e-132-78 | (1.068%) | (5.92%) | 1:6 | 407 | Pass | Pass |
| AX1e-132-82 | (0.350%) | (6.65%) | 1:20 | 537 | Pass | Pass |
| AX1e-132-83 | (0.230%) | (6.77%) | 1:30 | 466 | Pass | Pass |
| AX1e-132-84 | (0.175%) | (6.825%) | 1:40 | 549 | Pass | Pass |
| AX1e-132-91 | (0.350%) | (6.65%) | 1:20 | 531 | Pass | Pass |
| AX1e-132-92 | (0.230%) | (6.77%) | 1:30 | 433 | Pass | Pass |
| AX1e-132-93 | (0.175%) | (6.825%) | 1:40 | 543 | Pass | Pass |
| BX | CPC | Tween 20 | | | | |
| BX1e-132-79 | (0.350%) | (6.650%) | 1:20 | 456 | Pass | Pass |
| BX1e-132-80 | (0.230%) | (6.770%) | 1:30 | 466 | Pass | Pass |
| BX1e-132-81 | (0.175%) | (6.825%) | 1:40 | 473 | Pass | Pass |
| CX | CPC | P407 | | | | |
| CX1e-132-85 | (0.70%) | (6.30%) | 1:10 | 251 | Pass | Pass |
| CX1e-132-86 | (0.350%) | (6.65%) | 1:20 | 308 | Pass | Pass |
| CX1e-132-87 | (0.175%) | (6.825%) | 1:40 | 302 | Pass | Pass |

TABLE 5
Stability Results of Benzalkonium Chloride (BAC) Nanoemulsion Formulations

| | % Cationic | % Nonionic | | | Appearance | Particle Size |
|---|---|---|---|---|---|---|
| | Surfactant | Surfactant | Surfactant | Ave Mean | (Pass/Fail at | (Pass/Fail at |
| | in Neat | in Neat | Blend Ratio | Particle | 2 wks at 22° | 2 wks at 22° |
| Series | (Positive) | (Neutral) | (Cat/Non) | Size (nm) | and 40° C.) | and 40° C.) |
| AX | BC1 | Tween 80 | | | | |
| AX2e-130-40 | (1.0%) | (5.92%) | 1:6 | 448 | Pass | Pass |
| AX2e-130-41 | (3.49%) | (3.49%) | 1:1 | 321 | Pass | Pass |
| AX2e-130-42 | (6.0%) | (1.068%) | 6:1 | — | Fail | Fail |
| BX | BC1 | Tween 20 | | | | |
| BX2e-130-43 | (1.0%) | (5.92%) | 1:6 | 391 | Pass | Pass |
| BX2e-130-44 | (3.49%) | (3.49%) | 1:1 | 385 | Pass | Pass |
| BX2e-130-45 | (6.0%) | (1.068%) | 6:1 | — | Fail | Fail |
| CX | BC1 | P407 | | | | |
| CX2e-130-46 | (1.0%) | (5.92%) | 1:6 | 280 | Pass | Pass |
| CX2e-130-47 | (3.49%) | (3.49%) | 1:1 | — | Fail | Fail |
| CX2e-130-48 | (6.0%) | (1.126%) | 6:1 | — | Fail | Fail |

*NF = nanoemulsion did not form

In order to investigate the effect of a larger cationic surfactant polar head group, cocamidopropyl betaine (CAB) was selected. Cocamidopropyl betaine (CAB) has an HLB around 11, which indicates hydrophobicity balance between the polar head group and nonpolar tail. CAB formulated with Tween 80 and P407 formed stable emulsions, however CAB did not form a stable emulsion with Tween 20 or P188 as reported in Table 6. Cocamidopropyl betaine has a 12 carbon chain length combined with a more linear or larger polar head group. The optimization of the polar head and hydrophobic tail regions of the cationic surfactants appeared to be important for stability.

TABLE 6
Stability results of formulations with cocamidopropyl betaine (CAB)

| | % Cationic | % Nonionic | Surfactant | | Appearance | Particle Size |
|---|---|---|---|---|---|---|
| | Surfactant | Surfactant | Blend | Mean | (Pass/Fail at | (Pass/Fail at |
| Series | in Neat | Neat | (CPC/Nonionic | Particle | 2 wks at 22° | 2 wks at 22° |
| Lot # | (Positive) | (Nonionic) | Surfactant) | Size (nm) | and 40° C.) | and 40° C.) |
| | CAB | Tween 80 | | | | |
| AX9e-131-69 | (1.0%) | (5.92%) | 1:6 | 451.5 | Pass | Pass |
| | CAB | Tween 20 | | | | |
| BX9e-131-70 | (1.0%) | (5.92%) | 1:6 | NF* | Fail | Fail |
| | CAB | P407 | | | | |
| CX9e-131-71 | (1.0%) | (5.92%) | 1:6 | 329.9 | Pass | Pass |
| | CAB | P188 | | | | |
| OX9e-131-72 | (1.0%) | (5.92%) | 1:6 | NF* | Fail | Fail |

*NF = nanoemulsion did not form

To investigate the effect of a longer hydrophobic chain tail group, stearalkonium chloride (SAC) was selected. SAC's longer 18 carbon hydrophobic tail resulted in a lowering of the HLB for SAC as compared to BCL. Hence, SAC is less prone to migration into aqueous phase as compared to BCL (better residence at the oil/water interface). SAC has a polar head group with similar size to CPC or BCL. Both surfactants have the same structure of cationic polar head group. Stability assessments for stearalkonium chloride (SAC) are listed in Table 7.

TABLE 7
Stability results of stearalkonium chloride (SAC) nanoemulsion formulations.

| | % Cationic | % Nonionic | | | Appearance | Particle Size |
|---|---|---|---|---|---|---|
| | Surfactant | Surfactant #1 | Surfactant | Mean | (Pass/Fail at | (Pass/Fail at |
| | in Neat | in Neat | Blend Ratio | Particle | 2 wks at 22° | 2 wks at 22° |
| Series | (Positive) | (Neutral) | (Cat/Non) | Size (nm) | and 40° C.) | and 40° C.) |
| BX | SAC | Tween 20 | | | | |
| BX4e-132-68 | (1.068%) | (5.92%) | 1:6 | 499 | Pass | Pass |
| BX4e-132-69 | (3.49%) | (3.49%) | 1:1 | 480 | Pass | Pass |
| CX | SAC | P407 | | | | |
| CX4e-132-70 | (1.068%) | (5.92%) | 1:6 | 280 | Pass | Pass |
| CX4e-132-71 | (3.49%) | (3.49%) | 1:1 | 325 | Pass | Pass |

To investigate the effect of a dual hydrophobic chain tail group verses a single chain group, dioctadecyl dimethyl ammonium chloride (DODAC) was selected. Dioctadecyl dimethyl ammonium chloride (DODAC) has two C18 carbon chain tails. DODAC also have a smaller polar head group when compared to CPC. These surfactants were more difficult to formulate with various nonionic surfactants, especially the poloxamers, and various manufacturing alternations were made (e.g. elevation of temperature, addition of water in the neat phase, extended homogenization times). The mean particle size was larger for the DODAC formulation than for some other cationic surfactant formulations manufactured. Additionally, some of these formulations exhibited bi-modal particle size distributions as reported in Tables 8. However, some DODAC were deemed stable. The particle size distributions remained unimodal or bimodal throughout the stability evaluation.

TABLE 8
Stability results of formulations with DODAC.

| | % Cationic | % Nonionic | Surfactant | | Appearance | Particle Size |
|---|---|---|---|---|---|---|
| | Surfactant | Surfactant | Blend | Mean | (Pass/Fail at | (Pass/Fail at |
| Series | in Neat | in Neat | (CPC/Nonionic | Particle | 2 wks at 22° | 2 wks at 22° |
| Lot # | (Positive) | (Nonionic) | Surfactant) | Size (nm) | and 40° C.) | and 40° C.) |
| | DODAC | Tween 80 | | | | |
| AX7e-131-84 | (1.0%) | (5.92%) | 1:6 | 518.6 | Pass | Pass |
| AX7e-132-23 | (3.49%) | (3.49%) | 1:1 | 529.8 | Pass | Pass |
| AX7e-132-24 | (5.92%) | (1.0%) | 6:1 | 638.3 | Pass | Pass |
| | DODAC | Tween 20 | | | | |
| BX7e-131-85 | (1.0%) | (5.92%) | 1:6 | 516.3 | Pass | Pass |
| BX7e-132-25 | (3.49%) | (3.49%) | 1:1 | 519.0 | Pass | Pass |
| BX7e-132-26 | (5.92%) | (1.0%) | 6:1 | 604.0 | Pass | Pass |
| | DODAC | P407 | | | | |
| CX7e-131-86 | (1.0%) | (5.92%) | 1:6 | NF | Fail | Fail |
| CX7e-132-29* | (1.0%) | (5.92%) | 1:6 | 501.7 | Pass | Pass |
| CX7e-132-27* | (3.49%) | (3.49%) | 1:1 | 425.0 | Pass | Pass |
| CX7e-132-28 | (5.92%) | (1.0%) | 6:1 | 303.3 | Pass | Pass |
| | DODAC | Tyloxapol | | | | |
| DX7e-131-87 | (1.0%) | (5.92%) | 1:6 | 567.3 | Pass | Pass |
| | DODAC | Span 20 | | | | |
| HX7e-131-88 | (1.0%) | (5.92%) | 1:6 | NF | Fail | Fail |
| | DODAC | Span 80 | | | | |
| LX7e-131-89 | (1.0%) | (5.92%) | 1:6 | 549.8 | Pass | Pass |
| | DODAC | P188 | | | | |
| OX7e-131-90 | (1.0%) | (5.92%) | 1:6 | NF** | Fail | Fail |

*Alternative manufacturing process.
**NF = nanoemulsion did not form

Bio-loading screening protocol for the panel of novel nanoemulsion formulations.

Experiments were conducted in order to evaluate the potential impact of wound exudates or “bio-burden” on the stability and efficacy of antimicrobial NEs for application in multiple types of wounds. Wound exudates typically contain of fibrin, platelets, serum components, white blood cells and/or other types of mediators and debris associated with tissue injury, inflammation and repair. The presence of wound exudates or “bio-burden” at the site of topical application may impact the stability and antimicrobial efficacy of NEs of varying compositions depending on the type of wound. Therefore, experiments were conducted to evaluate the potential impact of human serum as a model to mimic the effects of bio-loading on NE stability and antimicrobial activity.

One focus was to develop a series of nanoemulsion formulations that could be used in a bioloading screening study to look at the effect of the bioloading of human serum proteins on the physical chemical properties of the nanoemulsions. These studies looked at the effect of human serum concentration on the physical integrity of the nanoemulsion droplets. The ratio of nanoemulsion and serum protein (or bio-load) was at a 1:1 ratio of nanoemulsion and to a range of serum concentrations in broth. It was also determined that concentration of EDTA in the nanoemulsion compositions is an important factor for combating bio-load effects and improved anti-microbial activity. The dilution factor of that material for high quality zeta potential measuring was determined. The percentage of serum in the broth solution was evaluated at 1.5%, 3.13%, 6.25% 12.5%, 25% and 50%. The dilution of those samples for zeta potential measure was optimized at 0.015% in 10 mM EDTA. The 10 mM concentration of EDTA was equivalent to that the external phase in the 1:1 nanoemulsion:serum broth mixture. The final bio-load protocol for testing nanoemulsion with serum protein was as follows:

Protocol for Bio-load Formulation Screening:
1. 30% nanoemulsion formulation containing 20 mM EDTA
2. Four serum percentages in broth: 6.25%, 12%, 25% and 50%
3. 1:1 ratio of 30% nanomeulsion to the serum/broth mixture
4. 0.015% Nanoemulsion/serum mixture in 10 mM EDTA for particle size analysis and zeta analysis.

TABLE 9
Nanoemulsion formulations screened in bio-load study.

| | Cationic | Nonionic | Surfactant Blend | | | | |
|---|---|---|---|---|---|---|---|
| Groupings | Surfactant | Surfactant | (CPC/Nonionic | | EDTA | Particle Size | |
| Purpose | Type | Type | Surfactant) | | (mM) | (PdI) | |
| 1 | Cetylpyridium | Tween 20 | 1:6 | (MF) | 20 | 172 | (0.074) |
| Effect of | chloride (CPC) | | 1:1 | (H) | 20 | 557 | (0.20) |
| Surfactant | | | 6:1 | (H) | 20 | 698 | (0.34) |
| Blend | | | | | | | |
| Ratio | | | | | | | |
| 2 | CPC | Tween 20 | 1:6 | (MF) | 20 | 172 | (0.074) |
| Effect of | Dioctadecyl | | 1:6 | (H) | 20 | 592 | (0.26) |
| Cationic | dimethyl | | | | | | |
| Surfactant | ammonium | | | | | | |
| | chloride | | | | | | |
| | (DODAC) | | | | | | |
| | BAC | | | | | | |
| 3 | CPC | Tween 20 | 1:6 | (MF) | 20 | 172 | (0.074) |
| Effect of | | P407 | 1:6 | (H) | 20 | 210 | (0.06) |
| Nonionic | | | | | | | |
| Surfactant | | | | | | | |

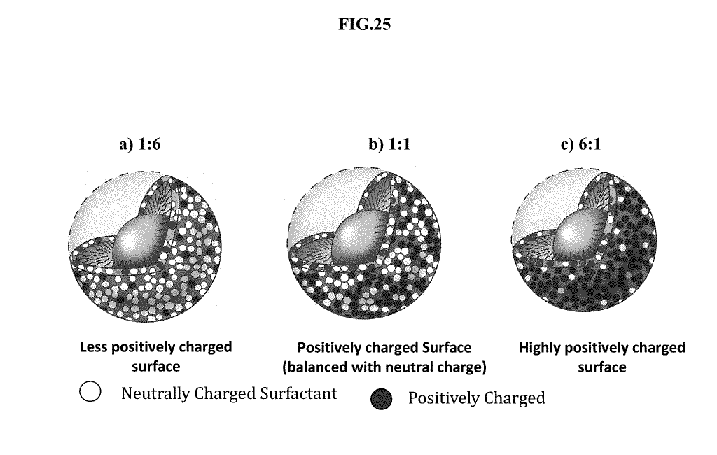

The effect of the surfactant blend ratio in three different CPC/Tween 20 formulations at a 1:6, 1:1, and 6:1 ratio, all containing 20 mM EDTA is shown in FIG. 25. The surfactant blend ratio affects the physical stability of the nanoemulsion droplets when in the presence of serum proteins. The larger proportion of cationic surfactant in the surfactant blend leads to less stable emulsion droplets when in the presence of the serum proteins. The size of the droplets increase with the increasing cationic surfactant in the surfactant blend. This indicated that, in some embodiments, there may be an optimal size and/or concentration of cationic surfactant needed in the blend to create stable droplets in the presence of serum proteins. The PdI follows the same trend; as the cationic surfactant is increased, the PdI becomes larger. When the PdI is larger than 0.3, bimodal and trimodel particle size distributions are present.

The effect of the surfactant blend ratio (e.g. 1:6, 1:1) in SAC/P407 compositions were also investigated in the bioload screening assay. The SAC/P407 composition with a 1:6 ratio was stable in all serum levels. The 1:1 composition was stable up to 50% serum level. This was the first time a 1:1 surfactant blend ratio was stable at higher serum levels. While an understanding of a mechanism is not necessary to practice the present invention, and while the invention is not limited to any particular mechanism, in some embodiments, increased stability is attributed to the longer carbon tail stabilizing the interface of the nanoemulsion droplets in combination with P407.

The SAC/P407 formulations are unimodal in particle size distribution. However, the SAC/Tween 20 compositions have bimodal distribution upon manufacturing. In particular, studies performed during development of embodiments of the invention showed that the SAC/Tween 20 compositions may be more stable than indicated by the change in mean size since the mean particle size accounts for both populations. The main peak in the size profile was around (550 nm) with a small peak around 5000 nm. Therefore, when a bimodal distributions of a nanoemulson of the invention is observed, identifying a corresponding PdI profile may be more informative (e.g., than other measured nanoemulsion characteristics) with regard to nanoemulsion biophysical properties (e.g., stability in serum). The SAC/Tween 20 at 1:1 ratio appeared to be less stable at the 50% serum level. The SAC/Tween 20 remained relatively unimodal and constant, indicating stability in serum.

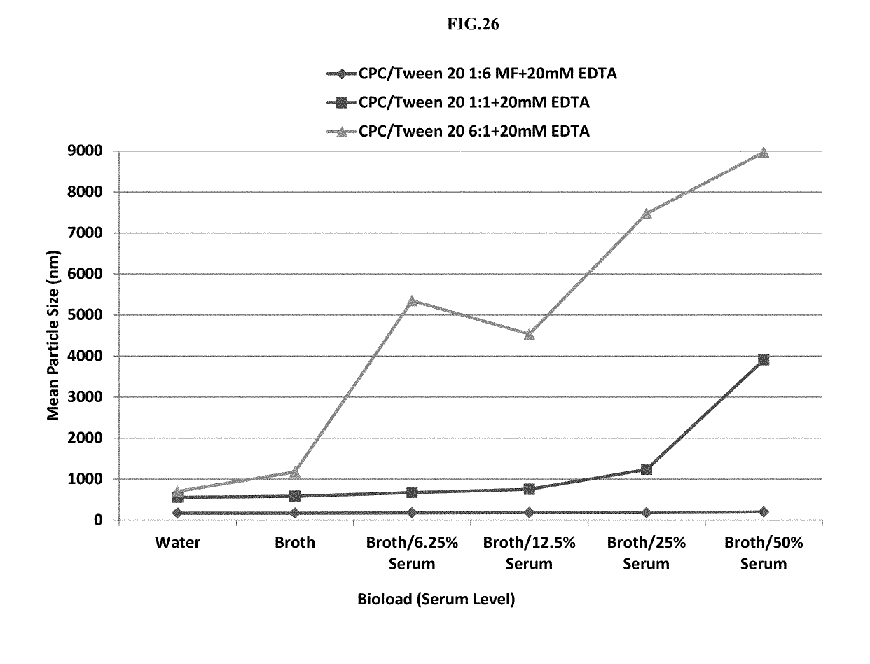

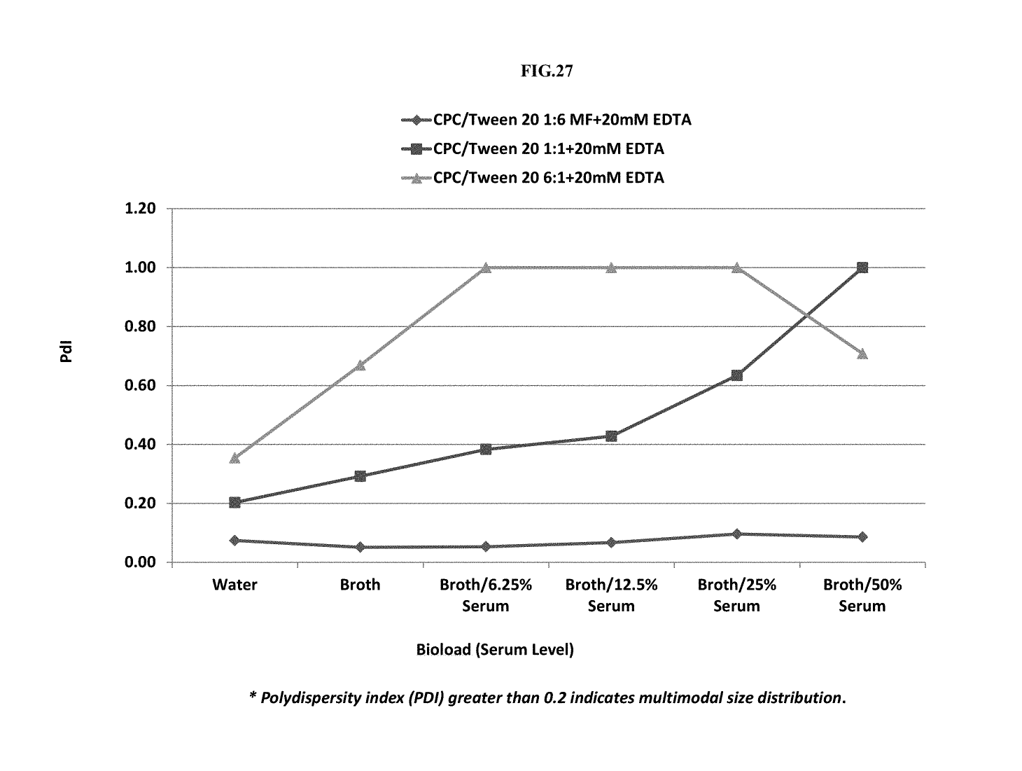

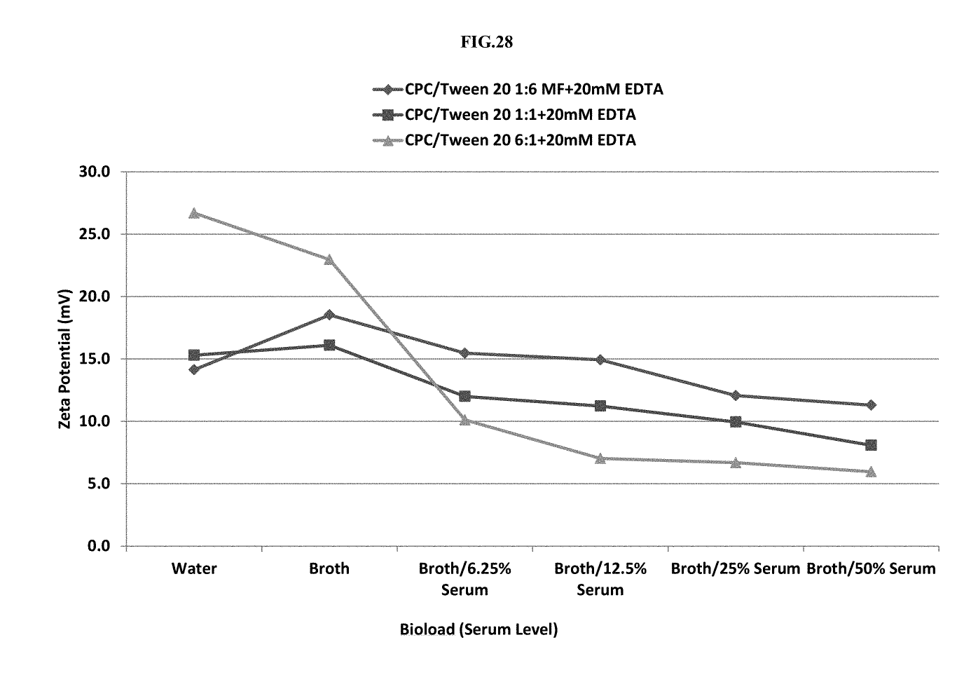

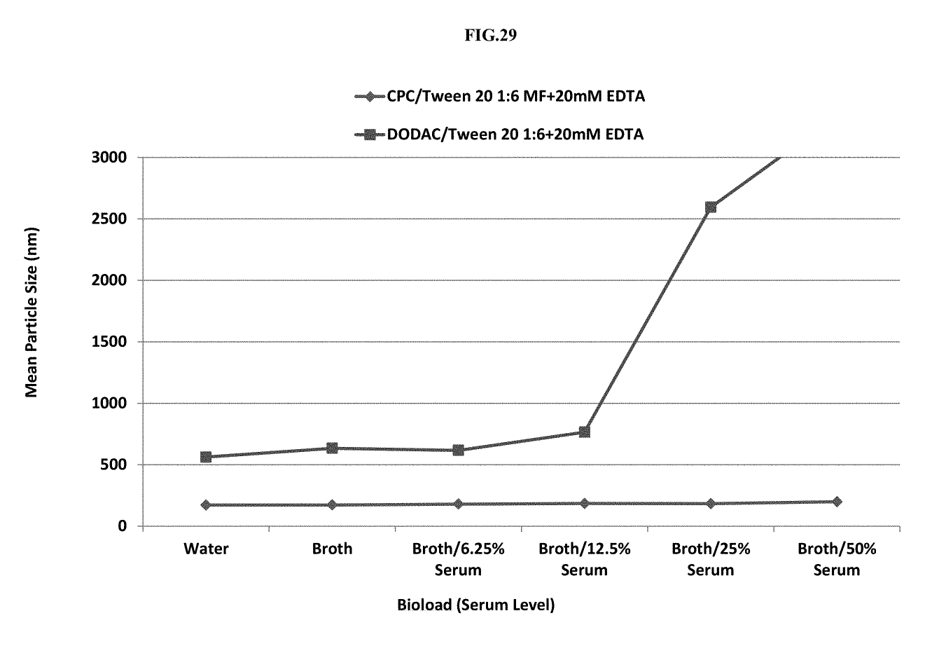

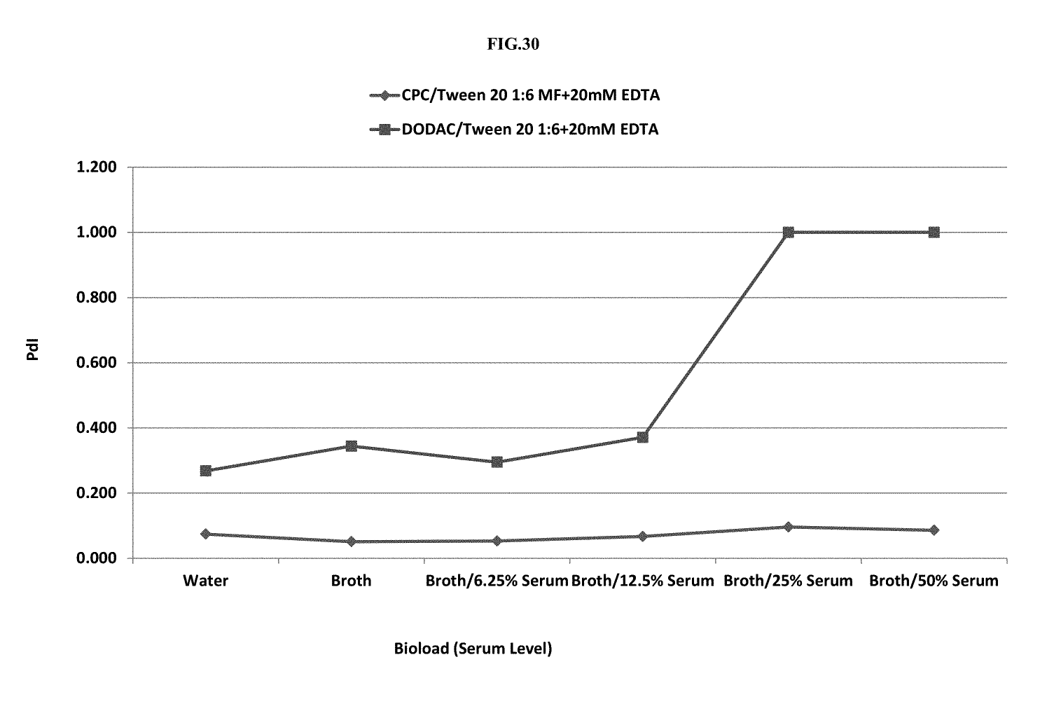

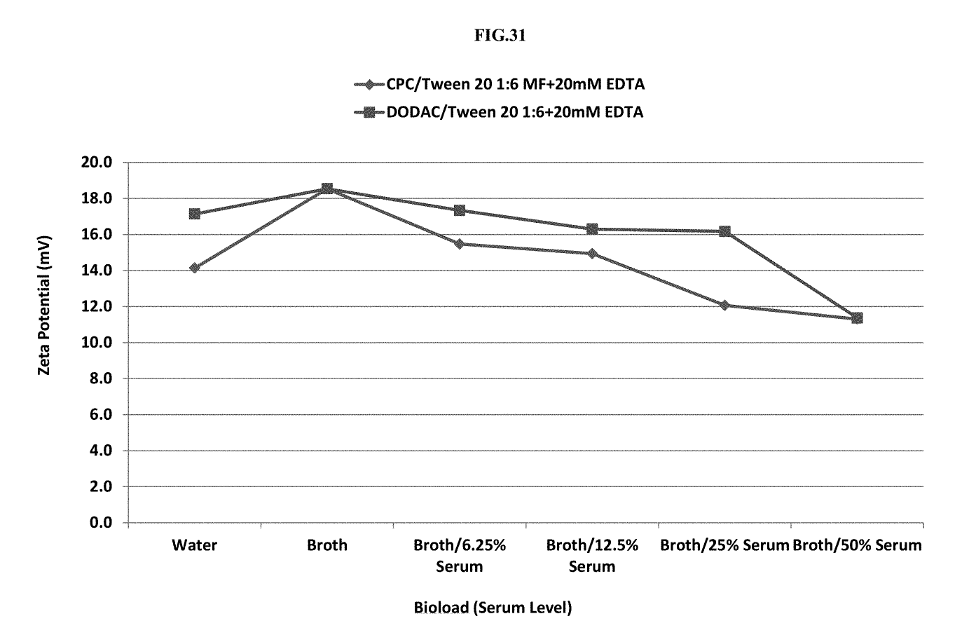

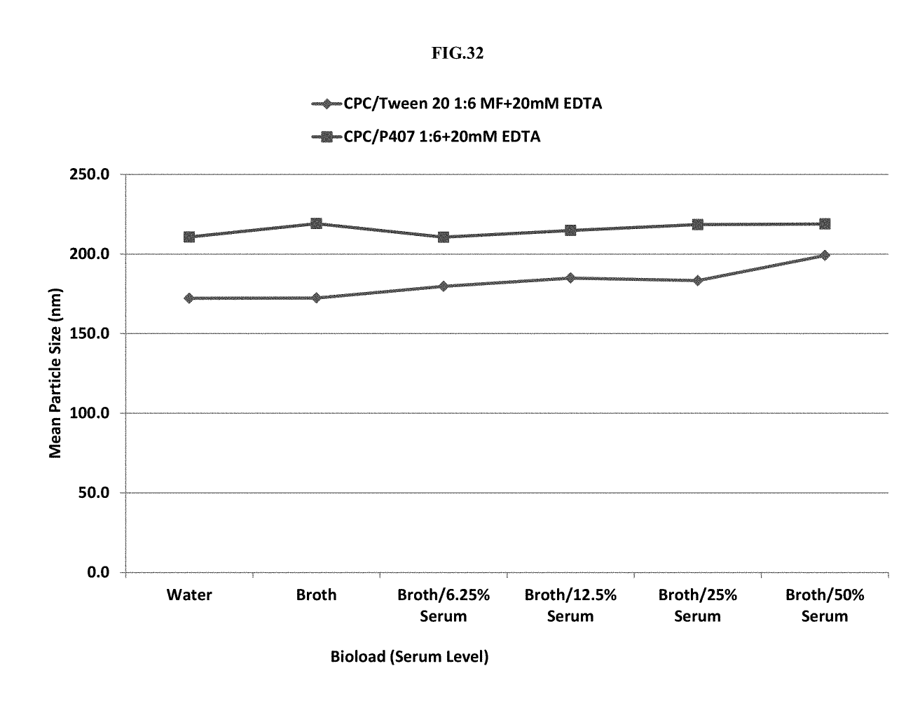

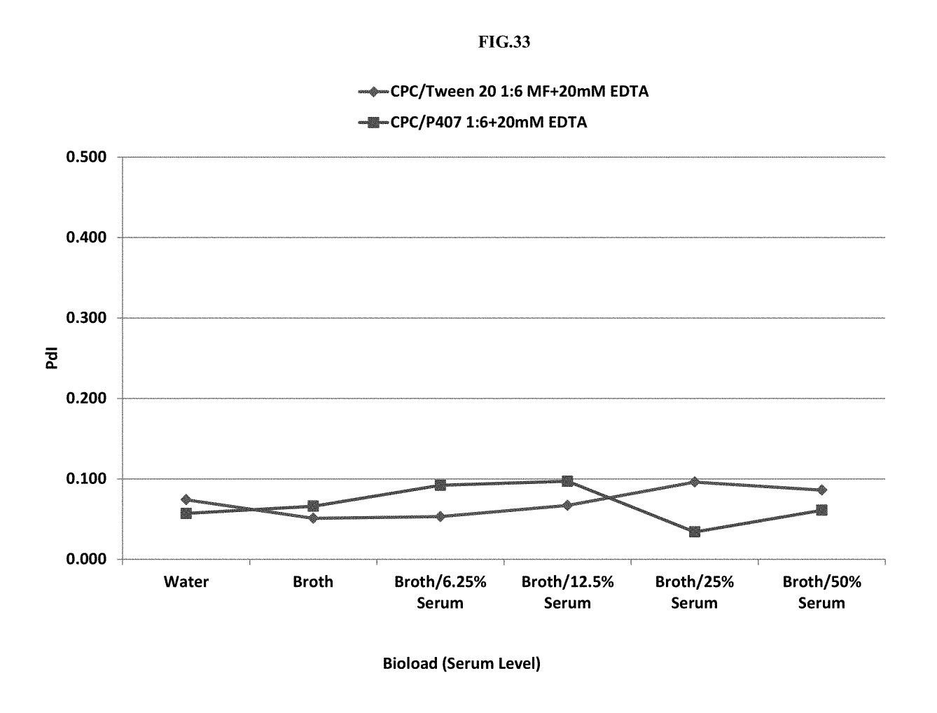

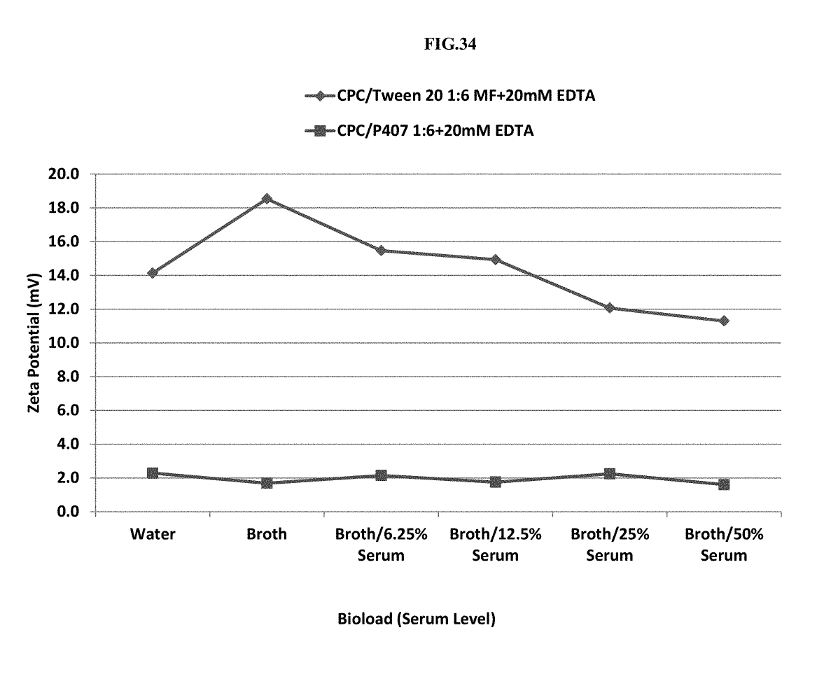

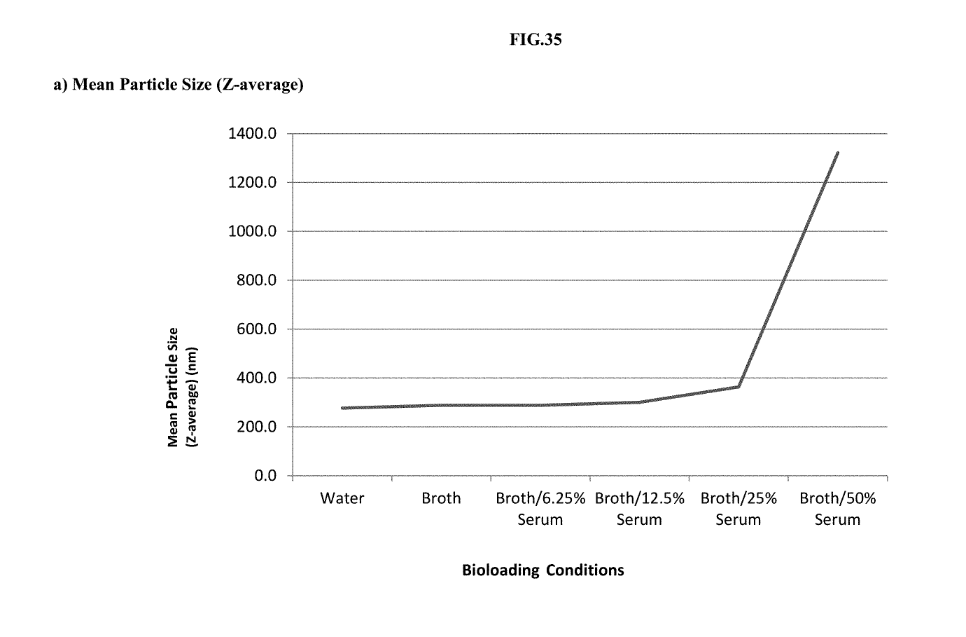

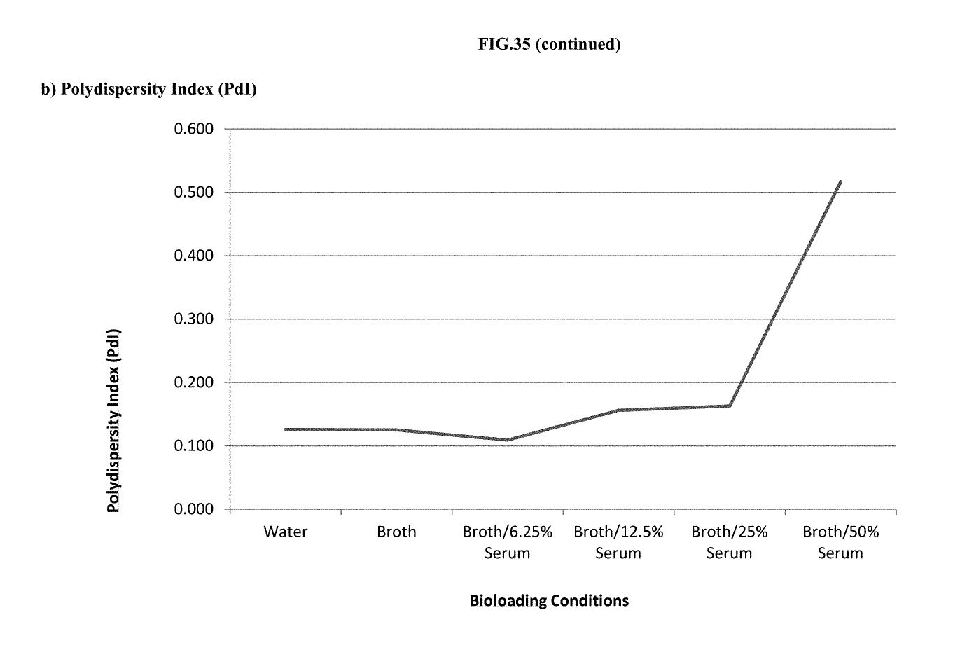

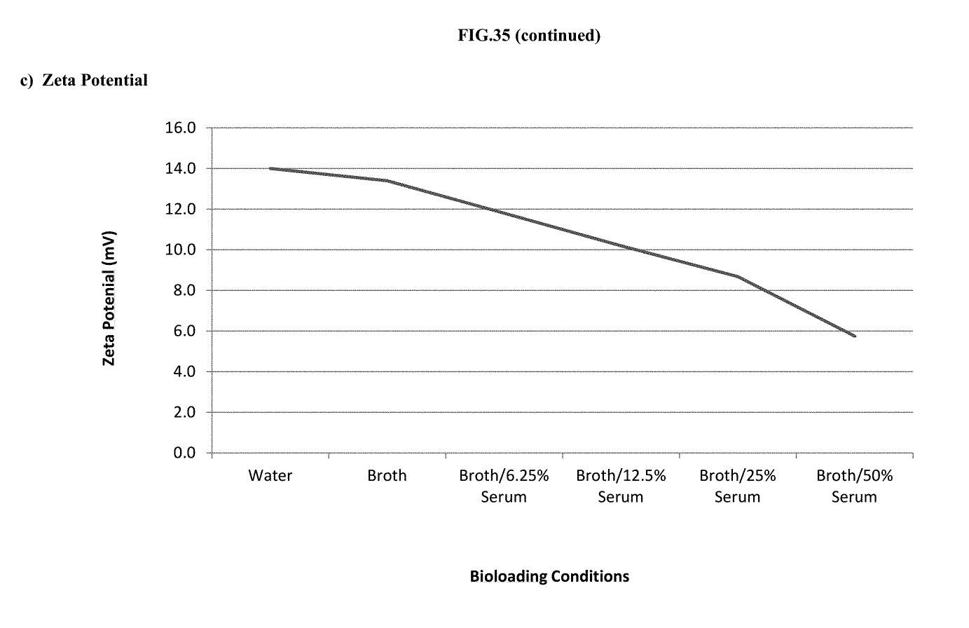

Zeta potential data obtained during development of embodiments of the invention was surprising. The formulation with the highest amount of cationic surfactant, 6:1, resulted in the highest zeta potential before the addition of the serum proteins. The 1:1 surfactant blend has less positive surface charge than the 6:1 blend. The 1:6 surfactant blend ratio resulted in positivity charged droplets but with the lowest zeta potential. The surface charge density is illustrated in FIG. 26. With the addition of serum proteins, all the formulations decreased in zeta potential, presumably due to complexion of the proteins with the positively charged nanoemulsion droplets. However, the 1:6 had the lowest decrease from 14.1 in water to 11.3 at the highest serum protein loading, while, the 6:1 resulted in the largest drop in zeta potential, 26.7 in water to 6 with the highest serum protein level. This indicated either an aggregation or destruction of nanoemulsion droplets when mixed with serum protein to form larger droplets with less overall positive surface charge. This event would lead to a reduction in the overall zeta potential.

The effect of the cationic surfactant was investigated. CPC or DODAC were formulated with Tween 20 at a surfactant blend ratio (cationic/nonionic) of 1:6 with 20 mM EDTA. The initial sizes of the nanoemulsion droplets were different. The particle size of CPC/Tween 20 was ˜180 nm, while DODAC/Tween 20 was ˜550 nm. It appeared that the CPC/Tween 20 formulation was more stable with higher serum protein concentration as compared to DODAC/Tween 20 with respect to mean particle size and PdI. The decreases in zeta potential were similar.

The effect of the nonionic surfactant was investigated. CPC was formulated at a 1:6 surfactant blend ratio (cationic:nonionic) with Tween 20 or P407. Table 3 shows a comparison between the chemistry of Tween 20 and P407. The mean particle size of the droplets of the Tween 20 and P407 were similar. Both formulations were stable at the serum protein levels tested. The zeta potential showed that the Tween 20 formulation had a higher overall zeta potential as compared to the P407 formulation. Also, the decrease was relatively stable for both formulations (Tween 20 14 to 11, P407 2 to 2). Since an overall positive surface charge of the nanoemulsion is highly desirable for killing, it was apparent that the P407 nonionic surfactant shielded the charge of CPC. As shown in Table 3, P407 has two very large polar head groups that would shield the charge. In some embodiments, low surface coverage of hydrophilic chains leads to a configuration where most chains are located closer to the particle surface (more protein bind at the surface). In the high surface coverage, the lack of mobility of the hydrophilic chains leads to a configuration where most of the chains are extended away from the surface (restrict binding of proteins to the surface) and also shielding the positive charge of CPC.

Results from benzalkonium chloride (BAC) formulation bioloading study are shown in FIG. 37. The benzalkonium chloride/tween 20 surfactant blend ratio was 1/3 with 20 mM EDTA in the external aqueous phase. The BAC formulation was stable with respect to mean particle size and PdI in 25% human serum. The zeta potential of the formulation decreased as the percent of human serum increased and retained its positive charge.

### Example 2

### Nanoemulsion and Telfa Absorption and Compatibility Study

Several of the nanoemulsions generated and initially characterized in Example 1 were utilized for further analysis.

Stability and compatibility of nanoemulsion with TELFA pad wound dressing was assessed. Experiments were conducted to determine stability and compatibility with TELFA for in vivo animal wound and burn models. TELFA (Kendall Co., Mansfield, Mass.) and TEGADERM HP (3M Health Care, St Paul, Minn.) were applied to prevent wound contamination and were used as a dressing in the in vivo experiments. Nanoemulsions tested are shown in Tables 10 and 11.

The burn wound was then redressed with TELFA and a TEGADERM occlusive dressing. The treatment and dressing change was repeated once, at 22 hours after burn injury.

Experiments were designed to determine the maximum absorption of the nanoemulsions, shown in Table 11, in a TELFA pad and the stability of the nanoemulsion formulations over time in contact with the TELFA pad. Briefly, TELFA was cut into 6 cm×6 cm areas and the weights recorded. The nanoemulsions (Table 11) were applied separately in excess and allowed to achieve maximum absorption in the TELFA and the weights of the saturated TELFA pad were recorded.

The stability conditions were 10 minutes at room temperature (˜25° C.), 2 hours at 35° C., 4 hours at 35° C., and 12 hours at 35° C. The nanoemulsion was extracted by squeezing the TELFA pad, followed by transfer of the nanoemulsion to a glass vial. The weight of each vial was measured and recorded. The sample analysis included: observation of physical appearance; measuring pH: particle size analysis (mean Z-average, PdI); % CPC or BAK (label claim); and % EDTA (label claim).

The specific experimental procedure for TELFA stability study was as follows:
1. Place petri dish and petri dish cover on balance and record weight.
2. Cut a Telfa in 6 cm×6 cm square and place into petri dish; cover and record weight. Subtract weight of petri dish from the weight of petri dish plus Telfa to obtain the weight of the Telfa alone.
3. Place 10 mL of nanoemulsion into petri dish. Record the total weight.
4. Incubate the Telfa with the 10 mL of nanoemulsion for the following duration and temperatures:
   10 minutes at 25° C.
   4 hours at 37° C.*
   12 hours at 37° C.* *Parafilm the edges of the petri dish when incubation time is longer than 10 minutes.
5. Remove Telfa from the petri dish and carefully remove excess nanoemulsion with a Kim wipe.
6. Weigh a new, clean petri dish and place the soaked Telfa in the dish and record the weight.
   To determine the maximum amount absorbed, subtract the petri dish weight from the total weight (with soaked Telfa).
7. Remove the non-absorbed nanoemulsion retained in the petri dish and place in 20 mL glass vial.
8. Weigh a 20 mL glass scintillation vial
9. Carefully squeeze at the nanoemulsion out of the Telfa using tongs, into scintillation vial. Record the weight of the nanoemulsion extracted.
10. Determine the following from the extracted nanoemulsion, non-absorbed nanoemulsion, and control nanoemulsion for the following: pH, particle size profile (mean Z-average, PdI, Dv 10, Dv 50, Dv 90), % CPC, % EDTA or % BAC.

As shown in Tables 12, 13 and 14, the nanoemulsion formulations tested were stable with respect to particle size, pH and % label claim for BAK or CPC and EDTA up to 12 hours at 35° C. (typical surface temperature of the skin). Surprisingly, there was no binding of BAK, CPC or EDTA to the TELFA pad. In Tables 12, 13 and 14, “*” indicated a recorded observation that the nanomeulsion appeared both a white and homogenous matter.

TABLE 10
Quantitative Composition of NB-201, NB-
401, and NB-401 Vehicle Formulations.

| | | NB-401 | NB-401 | NB-201 |
|---|---|---|---|---|
| | | (CPC/P407) | Vehicle (P407) | (BAC/Tween 20) |
| | | 1:6 | 0:6 | 1:6 |
| | | 10% NB-401 | 10% NB- | 10% NB-201 |
| | | 0.1% CPC | 401Veh | 0.2% BAC |
| Ingredients | Function: | (% w/w) | (% w/w) | (% w/w) |
| Sterile | Aqueous Diluent | 91.60 | 91.71 | 91.37 |
| Water (USP) | | | | |
| Soybean Oil | Hydrophobic oil | 6.279 | 6.279 | 6.279 |
| (USP) | | | | |
| (super-refined) | | | | |
| Dehydrated | Organic solvent | 0.673 | 0.673 | |
| Alcohol (USP) | | | | |
| (anhydrous | | | | |
| ethanol) | | | | |
| Glycerol | | | | 0.800 |
| Poloxamer 407 | Emulsifying agent | 0.592 | 0.592 | — |
| (NF) | | | | |
| Tween 20 | Emulsifying agent | | — | 0.592 |
| Cetylpyridinium | Emulsifying | 0.1068* | — | — |
| Chloride | agent, | | | |
| (USP) | preservative and | | | |
| | active | | | |
| Benzalkonium | Emulsifying | — | — | 0.2136 |
| Chloride | agent, | | | |
| (USP) | preservative and | | | |
| | active | | | |
| EDTA | Preservative | 0.744 | 0.744 | 0.744 |

| | | | |
|---|---|---|---|
| Total | 100.00% | 100% | 100.00% |

* = CPC potency adjusted for water content in the monohydrate

TABLE 11
Composition, mean particle size and polydipersity index
of exemplary nanoemulsion formulations (e.g., evaluated
in TELFA pad stability and compatibility study).

| | Type of Cationic/ | | |
|---|---|---|---|
| | Nonionic | Mean Particle | Polydispersity |
| Formulations | Surfactant | Size (nm) | (PdI) Index |
| NB-201 | BAC/Tween 20 | 257.0 ± 1.7 | 0.081 ± 0.031 |
| NB-402 | CPC/P407 | 212.1 ± 2.7 | 0.103 ± 0.025 |
| NE Vehicle | None/P407 | 336.2 ± 12.8 | 0.135 ± 0.034 |

TABLE 12
Stability and compatibility of 10% NB-402(CPC/P407) + 20 mM EDTA with TELFA pad.

| | | | | CPC (% | EDTA (% | Mean | | Amount | | |
|---|---|---|---|---|---|---|---|---|---|---|
| | Appear- | | | Label | Label | Particle Size | | Absorbed | % | % |
| Time | ance | Temp | pH | Claim) | Claim) | (Z-ave; nm) | PdI | (g) | Released | Retained |
| Initial | Pass* | RT | 4.82 | 103.6 | 104.2 | 212.1 ± 2.7 | 0.103 ± 0.025 | — | — | — |
| (Control) | | (~25° C.) | | | | | | | | |
| 10 minutes | Pass* | RT | 4.82 | 100.6 ± 0.8 | 105.6 ± 0.8 | 211.9 ± 3.1 | 0.089 ± 0.019 | 5.89 ± 0.12 | 48.7 ± 2.0 | 51.3 ± 2.0 |
| | | (~25° C.) | | | | | | | | |
| 2 hours | Pass* | 35° C./75% | 4.85 | 101.3 ± 0.2 | 107.1 ± 0.3 | 210.1 ± 2.5 | 0.084 ± 0.014 | 6.81 ± 0.22 | 43.7 ± 1.1 | 56.3 ± 1.1 |
| | | RH | | | | | | | | |
| 12 hours | Pass* | 35° C./75% | 4.89 | 105.5 ± 0.3 | 111.3 ± 0.1 | 211.2 ± 0.1 | 0.084 ± 0.015 | 5.68 ± 0.28 | 44.6 ± 0.07 | 55.3 ± 0.1 |
| | | RH | | | | | | | | |

TABLE 13
Stability and compatibility of 10% NB-201(BAK/Tween 20) + 20 mM EDTA with TELFA pad.

| | | | | BAK (% | EDTA (% | Mean | | Amount | | |
|---|---|---|---|---|---|---|---|---|---|---|
| | Appear- | | | Label | Label | Particle Size | | Absorbed | % | % |
| Time | ance | Temp | pH | Claim) | Claim) | (Z-ave; nm) | PdI | (g) | Released | Retained |
| Initial | Pass* | RT | 4.76 | 95.7 | 103.6 | 257.0 ± 1.7 | 0.081 ± 0.031 | — | — | — |
| (Control) | | (~25° C.) | | | | | | | | |
| 0 minutes | Pass* | RT | 4.77 | 97.4 ± 07 | 105.9 ± 0.5 | 256.9 ± 2.5 | 0.082 ± 0.016 | 6.38 ± 0.30 | 60.0 ± 4.9 | 40.0 ± 4.9 |
| | | (~25° C.) | | | | | | | | |
| 2 hours | Pass* | 35° C./75% | 4.77 | 101.1 | 107.6 | 258.7 ± 0.64 | 0.086 ± 0.014 | 6.14 | 50.0 | 50.0 |
| (n = 1) | | RH | | | | | | | | |
| 4 hours | Pass* | 35° C./75% | 4.77 | 96.8 | 107.5 | 255.3 ± 0.2 | 0.081 ± 0.010 | 6.25 | 55.5 | 44.5 |
| (n = 1) | | RH | | | | | | | | |
| 12 hours | Pass* | 35° C./75% | 4.74 | 102.3 ± 2.1 | 109.8 ± 0.2 | 259.5 ± 4.2 | 0.084 ± 0.012 | 5.76 ± 0.20 | 49.8 ± 2.1 | 50.1 ± 2.1 |
| | | RH | | | | | | | | |

TABLE 14
Stability and compatibility of vehicle

| | | | | CPC (% | EDTA (% | Mean | | Amount | | |
|---|---|---|---|---|---|---|---|---|---|---|
| | Appear- | | | Label | Label | Particle Size | | Absorbed | % | % |
| Time | ance | Temp | pH | Claim) | Claim) | (Z-ave; nm) | PdI | (g) | Released | Retained |
| Initial | Pass* | RT | 4.93 | 0 | 104.2 | 336.2 ± 12.8 | 0.135 ± 0.034 | — | — | — |
| (Control) | | (~25° C.) | | | | | | | | |
| 10 | Pass* | RT | 4.89 | 0 | 107.6 ± 1.0 | 332.4 ± 2.8 | 0.132 ± 0.021 | 6.61 ± 0.021 | 8.2 ± 0.1.7 | 41.8 ± 0.1.7 |
| minutes | | (~25° C.) | | | | | | | | |
| 4 horns | Pass* | 35° C./75% | 4.94 | 0 | 108.9 ± 1.3 | 333.7 ± 4.7 | 0.140 ± 0.030 | 6.26 ± 0.13 | 49.7 ± 0.3 | 50.3 ± 0.3 |
| | | RH | | | | | | | | |
| 12 hours | Pass* | 35° C./75% | 4.93 | 0 | 113.8 ± 1.1 | 336.2 ± 4.8 | 0.148 ± 0.019 | 6.61 ± 0.44 | 45.2 ± 3.3 | 54.7 ± 3.3 |
| | | RH | | | | | | | | |

To achieve a target dosing volume per surface area of skin of 1004/cm2, 3.6 mL (36004) was sprayed over the skin surface area using a template to achieve a 100₁11/cm2 to the wound area. The maximum amount absorbed by the TELFA pad was approximately 6 mL, and squeezing the TELFA pad released about 50%, leaving 3 mL trapped inside the TELFA pad. This data indicated that at least around 3 mL of formulation should be applied to the 6 cm×6 cm TELFA pad to prevent wicking away of the sprayed nanoemulsion from the wound area.

New Cream Nanoemulsion Formulations

The liquid formulation was amenable to application with a sprayer (e.g., for immediate treatment after injury). Further experiments were performed in an effort to determine if additional formulations (e.g., cream formulations) could be generated for use in covering a wound (e.g., prior to application of a dressing, bandage or other covering).

One formulation strategy tested for cream nanoemulsion formulations was to assess the effect that different cationic surfactants, non-ionic surfactants and the surfactant blend ratio have on stability. In previous experiments, the surfactant blend ratio had been determined to participate in cytotoxicity properties of the nanoemulsions. In vitro findings also indicated that the concentration of the cationic surfactant was also a factor of cytotoxicity. Thus, experiments were conducted to determine if the surfactant blend ratio could be altered and utilized to generate effective cream nanoemulsion formulations.

A 10% nanoemulsion concentration was compared to 80% nanoemulsion at varying surfactant blend ratios as described in Table 15, below.

As shown in Table 15, a 10% nanoemulsion formulation with a surfactant blend ratio of 1/3 was compared to an 80% NE formulation with a surfactant blend ratio of 1/24. The 10% nanoemulsion formulation with a surfactant blend ratio of 1/3 has a cationic surfactant concentration similar to the 80% nanoemulsion with a surfactant blend ratio of 1/24, roughly 0.2% BAK. These formulations displayed different mean particle sizes and number of nanoemulsion droplets (See Table 16). It was also determined that nanoemulsions with larger mean droplet sizes contain fewer numbers of droplets as compared to nanoemulsions with smaller mean droplet sizes at the same % BAK (See Table 16). However, when the % nanoemulsion was increased the total surface area of droplets increased by almost 9 fold. While and understanding of a mechanism of action is not needed to practice the present invention, and while the present invention is not limited to any particular mechanism of action, in some embodiments, an increase in the total surface area of nanoemulsion droplets impacts the biological/biophysical properties of the nanoemulsion (e.g., an increase in the total surface area of the nanoemulsion droplet increases the ability of the nanoemulsion to inhibit the progression of a partial thickness burn wound to a full thickness burn wound and/or or increases the anti-microbial properties of the nanoemulsion when applied to a surface (e.g., a burn wound surface)).

TABLE 15
Comparison of the composition of the liquid and cream formulations of NB-201.

| | | 10% NB-201 | 80% NB-201 | |
|---|---|---|---|---|
| | | Lotion | Cream | 10% NB-201 |
| | | (BAC/Tween20) | (BAC/P407) | Lotion |
| | | 1:3 | 1:24 | Placebo |
| Excipients | Function | (% w/w) | (% w/w) | (Tween 20) |
| Purified Water | Aqueous Diluent | 90.027 | 34.924 | 90.227 |
| (USP) | | | | |
| Soybean Oil (USP) | Hydrophobic oil | 6.279 | 50.232 | 6.279 |
| Edetate Disodium | Preservative | 1.894* | 1.894* | 1.894* |
| Dihydrate (USP) | | | | |
| Glycerol (NF) | Organic solvent | 1.008 | — | 1.008 |
| Ethanol (USP) | Organic solvent | — | 8.00 | — |
| Tween 20 (NF) | Emulsifying | 0.592 | — | 0.592 |
| | agent | | | |
| Poloxamer 407 | Emulsifying | — | 4.736 | — |
| (NF) | agent | | | |
| Benzalkonium | Emulsifying | 0.200 | 0.2136 | — |
| Chloride (USP) | agent, | | | |
| | preservative | | | |

| | | | |
|---|---|---|---|
| Total | 100.00% | 100.00% | 100% |

*50 mM EDTA

TABLE 16
Comparison of liquid and cream Formulations ofNB-201:Surfactant blend ratio, concentration
of cationic surfactant, particle size and number of droplets/mL.

| | Cationic | | | | | | # of Droplets | Total |
|---|---|---|---|---|---|---|---|---|
| | Surfactant/ | | | | Conc. Cat | Mean | (Based on Mean | Surface |
| Dosage | Non-ionic | | | % Cationic | Surfantant | Particle | Particle size & | area in |
| Form | surfactant | Blend Ratio | % NE | Surfactant | (μg/mL) | Size (nm) | % NE) | 1 ml |
| NB-201 | BAK/Tween 20 | 1/3 | 10 | 0.200 | 2000 | 257 | 7.1E+12 | 14,661 |
| Lotion | | | | | | | | |
| NB-201 | BAK/P407 | 1/24 | 80 | 0.214 | 2140 | 237 | 7.2E+13 | 127,189 |
| Cream | | | | | | | | |

Several of the nanoemulsion formulations described and characterized herein were oil-in-water (o/w) emulsion with a mean droplet diameter ranging from 180 to 260 nm. Benzalkonium chloride (BAC) and Cetylpyridinium chloride (CPC) are cationic surfactants that both reside at the interface between the oil and water phases. The hydrophobic tail of the surfactant distributes in the oil core and its polar cationic head group resides in the water phase. The corresponding placebo formulations without the cationic surfactant ranged in particle size from 360-490 nm. Thus, removing the cationic surfactant affected the particle size of the droplets.

Benzalkonium chloride is used as a preservative in pharmaceuticals and personal care products such as eye, ear and nasal drops. The greatest biocidal activity is associated with the C12 dodecyl and C14 myristyl alkyl derivatives. The mechanism of bactericidal/microbicidal action is thought to cause dissociation of cellular membrane lipid bilayers, which compromises cellular permeability controls and induces leakage of cellular contents.

Cetylpyridinium chloride (CPC) is a cationic quaternary ammonium compound in some types of mouthwashes, toothpastes, lozenges, throat sprays, breath sprays, and nasal sprays. It is an antiseptic that kills bacteria and other microorganisms.

From data accumulated during development of embodiments of the invention, the BAC formulation (NB-201) outperformed the CPC based (NB-402) formulation with respect to killing bacteria. While an understanding of a mechanism is not necessary to practice the present invention, and while the invention is not limited to any particular mechanism, in some embodiments, this is attributed to the varying chain lengths of BAC. BAC has 4 chain lengths and the length of each chain affects bacterium differently. The percentage of chain lengths used was: C12 (5%); C14 (60%); C16 (30%); C18 (5%). For example, C12 was best for killing fungi; C14 for gram (+); C16 for gram (−). CPC has a chain length of C16 (100%) in comparison. Also, the amount of cationic BAC used was a total of 0.2% for BAC, in comparison to 0.1% for CPC. The size of the droplets was similar for both formulations containing BAC or CPC. Thus, as described herein, removing the cationic surfactant from the composition had a significant effect on the killing of bacteria.

### Example 3

### Antimicrobial Activity of Nanoemulsion Formulations

Experiments were performed in order to test the microbicidal activity of nanoemulsion formulations described herein against a wide range of bacteria.

TABLE 17
Comparison of the Composition of the Liquid and
Cream Formulations of NB-201 containing BAK

| | | | NB-201 | |
|---|---|---|---|---|
| | | NB-201 Lotion | Cream | |
| | | Theoretical | Theoretical | NB-201 Lotion |
| Excipients | Function | (% w/w) | (% w/w) | Placebo |
| Purified Water | Aqueous Diluent | 90.027 | 34.924 | 90.227 |
| (USP) | | | | |
| Soybean Oil (USP) | Hydrophobic oil | 6.279 | 50.232 | 6.279 |
| Edetate Disodium | Preservative | 1.894 | 1.894 | 1.894 |
| Dihydrate (USP) | | | | |
| Glycerol | Organic solvent | 1.008 | — | 1.008 |
| Ethanol | Organic solvent | — | 8.00 | — |
| Tween 20 (NF) | Emulsifying | 0.592 | — | 0.592 |
| | agent | | | |
| Poloxamer 407 (NF) | Emulsifying | — | 4.736 | — |
| | agent | | | |
| Benzalkonium | Emulsifying | 0.200 | 0.2136 | — |
| Chloride (USP) | agent, | | | |
| | preservative and | | | |
| | active | | | |

| | | | |
|---|---|---|---|
| Total | 100.00% | 100.00% | 100% |

TABLE 18
Comparison of Lotion and Cream Formulations of NB-201 and NB-402:Surfactant
Blend Ratio, Concentration of Cationic Surfactant and Particle Size.

| | | Cationic | | | | | |
|---|---|---|---|---|---|---|---|
| | | Surfactant/ | | | | [Cationic | Mean |
| | | Non-ionic | Surfactant | | % Cationic | surfactant] | Particle |
| Dosage Form | LOT # | surfactant | Blend Ratio | % NE | Surfactant | (μg/mL) | Size (nm) |
| NB-201 | | | | | | | |
| NB-201 Lotion | BX2g-315x30 | BAK/Tween 20 | 1/3 | 10 | 0.200 | 2000 | 257 |
| Lotion (NB-201 | BX0g-315x31 | Tween 20 | 0/6 | 10 | 0 | 0 | 483 |
| Placebo) | | | | | | | |
| NB-201 Cream | CX2e-195x55 | BAK/P407 | 1/24 | 80 | 0.214 | 2140 | 237 |

TABLE 19
Comparison of liquid and cream Formulations ofNB-201:Surfactant blend ratio,
concentration of cationic surfactant, particle size and number of droplets/mL

| | | Cationic | | | | | | # of Droplets | Total |
|---|---|---|---|---|---|---|---|---|---|
| | | Surfactant/ | | | | [Cationic | Mean | (Based on Mean | Surface |
| Dosage | | Non-ionic | Surfactant | | % Cationic | surfactant] | Particle | Particle size & | area in |
| Form | LOT # | surfactant | Blend Ratio | % NE | Surfactant | (μg/mL) | Size (nm) | % NE) | 1 ml |
| NB-201 Lotion | BX2g-315x30 | BAK/Tween 20 | 1/3 | 10 | 0.200 | 2000 | 257 | 7.1E+12 | 14,661 |
| NB-201 Cream | CX2e-195x55 | BAK/P407 | 1/24 | 80 | 0.214 | 2140 | 237 | 7.2E+13 | 127,189 |

The MICs of NB-201 lotion, cream and control were determined by using a modification of the Clinical and Laboratory Standards Institute (CLSI)-approved microtiter serial dilution method (Clinical and Laboratory Standards Institute. 2006. Methods for dilution antimicrobial susceptibility tests for bacteria that grow aerobically. Approved standard M7-A7, 7th ed. Clinical and Laboratory Standards Institute, Wayne, Pa.). The formulations were diluted to a concentration of 2 mg/ml (of CPC) in MH broth supplemented with 7% NaCl and 20 mM EDTA. Serial twofold dilutions of this preparation were made in unsupplemented MH broth and aliquoted into 96-well flat-bottom microtiter plates (100 μl/well). Bacteria from overnight growth on MH agar were suspended in MH broth to a 0.5 McFarland turbidity standard (absorbance of 0.08 to 0.13 at 625 nm), further diluted 1:100 in MH broth, and added (5 μl/well) to the formulations serial dilution wells. Appropriate controls, including wells with bacteria but no formulation and wells with formulations dilutions but no bacteria, were included on each plate. Microtiter plates were shaken briefly, and 1 μl was removed from wells containing bacteria but no NB-401, diluted in 1 ml of MH broth, plated onto MH agar (100 μl), and incubated for 24 to 48 h at 37° C. to confirm that initial inoculums were ≥10⁵ CFU. Microtiter plates were then incubated at 37° C. without shaking. To determine minimal bactericidal concentrations (MBCs), 10 μl was removed from each well after overnight growth, spread onto MH agar, and incubated at 37° C. Colonies were enumerated 24 h later, and MBCs were recorded as the formulation concentrations with a 3-log decrease in CFU/ml compared to the initial inoculums. Because the formulations are opaque, 10 μl of resazurin (R&D Systems, Minneapolis, Minn.) was added to each well, and microtiter plates were shaken briefly, covered with foil, and incubated at 37° C. without shaking. Resazurin, a nonfluorescing blue dye, is reduced to resorufin, a fluorescing pink dye, in the presence of actively metabolizing cells. Therefore, MICs were recorded the next day as the lowest concentrations of formulations in which the wells remained blue. MIC results were further quantified by measuring the fluorescence generated by the reduction product resorufin on a spectrofluorometer at 560 nm excitation/590 nm emission. The change in metabolic activity for treated bacteria was calculated as follows: (fluorescence of the visual MIC well—fluorescence of the well with the equivalent concentration of the formulations without bacteria)/(fluorescence of the well containing bacteria but no formulation—fluorescence of the well containing medium only)×100 (See, e.g., Taneja and Tyagi. 2007. J. Antimicrob. Chemother. 60:288-293.).

TABLE 20
Range of MICs for two NB-201 BAC nanoemulsion formulations

| | | | | | Staphylococcus |
|---|---|---|---|---|---|
| | Acinetobacter | Klesiella | Enterococcus | | aureus, |
| | baumanii | pneumonia | spp., vancomycin | Pseudomonas | methicillin |
| NB-201 | (3) | (5) | resistant (5) | aeruginosa (5) | resistant (3) |
| NB-201 Cream | 1:4 | 1:1-1:8 | 1:16 | 1:1-1:4 | 1:256-1:512 |
| NB-201 Lotion | 1:2-1:4 | >1:1-1:8 | 1:16 | >1:1-1:8 | 1:256-1:512 |
| NB-201 | 1:8-1:16 | <1:1-1:2 | 1:1-1:16 | 1:1 | 1:16 |
| Vehicle | | | | | |
| (Control) | | | | | |

Table 20 summarizes the antimicrobial activity of BAC/P407 against 21 strains representing five species.

### Example 4

### Bacterial Wound Infection and Partial-Thickness Burn Injury Studies in Rats

Materials and Methods.

Reagents. Nanoemulsions described in Example 1 above (NB-201, NB-402 and NB-402 placebo; Table 10) were manufactured by and obtained from NANOBIO Corporation (Ann Arbor, Mich.). Two different cationic surfactants were used in the rat burn model. Benzalkonium chloride (BAC) was the cationic surfactant incorporated into the NB-201 formulation with Tween 20 as the nonionic surfactant (See Example 1). Cetylpyridium chloride (CPC) was the cationic surfactant incorporated into the NB-402 formulation with Poloxamer 407 (P407) as the nonionic surfactant (See Example 1). The nanoemulsion vehicle (NE vehicle) was prepared in a similar fashion, without incorporation of any cationic surfactant and P407 as the nonionic surfactant. The surfactants, both cationic and nonionic, reside at the interface between the oil and water phases. The hydrophobic tail of the surfactant distributes in the oil core and its polar head group resides in the water phase. Unless otherwise indicated, all other reagents were purchased from SIGMA-ALDRICH (St. Louis, Mo.).

Animals. Male specific pathogen-free Sprague-Dawley rats (Harlan, Indianapolis, Ind.) weighing approximately 250 to 300 g were used. The experiment was performed in accordance with the National Institutes of Health (NIH) guidelines for care and use of animals. Approval for the experimental protocol was obtained from the University of Michigan Animal Care and Use Committee.

Burn model. Rats were anesthetized with a 40 mg/kg intraperitoneal (ip) injection of sodium pentobarbital (Nembutal; ABBOTT Laboratories, North Chicago, Ill.). Dorsal hair was closely clipped and then removed using depilatory cream (NAIR; Church & Dwight Inc., Princeton, N.J.). Partial-thickness scald burn injury of 20% of the total body surface area was achieved by placing the exposed skin of the rat in a 60° C. water bath for 25 seconds. An occlusive dressing of sterile TELFA (Kendall Co., Mansfield, Mass.) and TEGADERM HP (3M HealthCare, St Paul, Minn.) was applied to prevent wound contamination. During experiments, each rat was singly housed and received 0.01 mg/kg buprenorphine subcutaneously at the time of burn and at 8 h, 16 h and 24 h for post-burn for pain control.

Local wound treatment. Each experimental group underwent burn followed by bacterial innoculation. At 6 hours after burn injury, animals were anesthetized with inhaled isoflurane. The occlusive dressing and TELFA was removed. NB-201, NB-402, vehicle control or sterile saline was applied in a uniform fashion to the burn wound surface using a spray bottle. The burn wound was then redressed with TELFA and a TEGADERM occlusive dressing. This treatment and dressing change was repeated at 14 and 22 hours after burn injury (Figure Model 1). In a separate set of experiments, we studied burn wound healing. Experimental groups consisted of burn wound only without bacterial innoculation. +saline, burn+vehicle controle, burn+NB-201, and burn+NB-402. The occlusive dressing and Telfa were changed at 8 h, 16 h, 24 h, 36 h and 48 h (Figure Model 2). Pictures were taken at the time of each dressing change.

Tissue harvest. At 30 or 72 hours after thermal injury, the animals were euthanized, and skin tissue samples were harvested employing standard sterile techniques. Skin samples were used immediately or frozen in liquid nitrogen.

Quantitation of bacterial wound infection. A 100 mg piece of excised skin tissue was mechanically homogenized in 1 mL of 0.9% NaCl. This homogenate was then further diluted with 9 mL of sterile saline. Serial dilutions were performed, and skin homogenates were plated in triplicate on blood agar plates (Becton Dickinson). Culture plates were incubated for 24 hours at 37° C., and CFUs counted.

Quantitation of soluble mediators by ELISA. A 100 mg piece of excised skin tissue was mechanically homogenized in 1 mL of 0.9% NaCl containing 0.01% of Triton X (Roche) and complete protease inhibitors cocktail (Complete X, ROCHE, Indianapolis, Ind.). This homogenate was then centrifuged at 3000 g for 5 minutes at +4° C. and used for ELISA. Rat cytokines and chemokines were measured by sandwich enzyme-linked immunosorbent assay (ELISA) using DUOSETS from R&D Systems Inc. (Minneapolis, Minn.). Rat myeloperoxidase ELISA kit was from Hycult biotech (Plymouth Meeting, PA). For rat MPO assay, skin tissue was homogenized in 1 mL of 0.9% NaCl containing complete protease inhibitors cocktail (ROCHE).

Histology. Fresh 4 mm full thickness skin tissue biopsies were fixed in 10% buffered formalin and embedded in paraffin. Sections 4 μm thick were sliced and affixed to slides, deparaffinized, and stained with hematoxylin and eosin to assess morphological changes. To evaluate neutrophil infiltration into the burn wound we counted neutrophils within a 1 mm xlmm microscope grid at high power (40× magnification) for six fields per slide of full thickness skin and an average value of cells/mm3 was determined for each burn wound sample. Skin histology samples were scored for burn injury by an independent and blinded pathologist using the following system: distribution of cellular infiltrate (0=none, 1=focal, 2=multifocal, 3=locally extensive, 4=multifocal and locally extensive, 5=diffuse), inflammation severity (0=none, 1=mild, 2=moderate, 3=severe), infiltrate type (0=none, 1=acute, 2=subacute, 3=chronic), necrosis (0=none, 1=minimal, 2=moderate, 3=severe). A final score was computed by summing the scores in each subdivision.

Statistical methods. All statistical analysis and graph(s) were performed using GRAPHPAD Prism software (version 5.0; GRAPHPAD Software, La Jolla, Calif.). Results are presented as mean values±the SEM unless otherwise noted. Continuous variables were analyzed using 1 way ANOVA and Newman-Keuls multiple comparison. Statistical significance was defined as a P value<0.05.

Results.

Nanoemulsion Treatment Reduces Dermal P. aeruginosa Infection

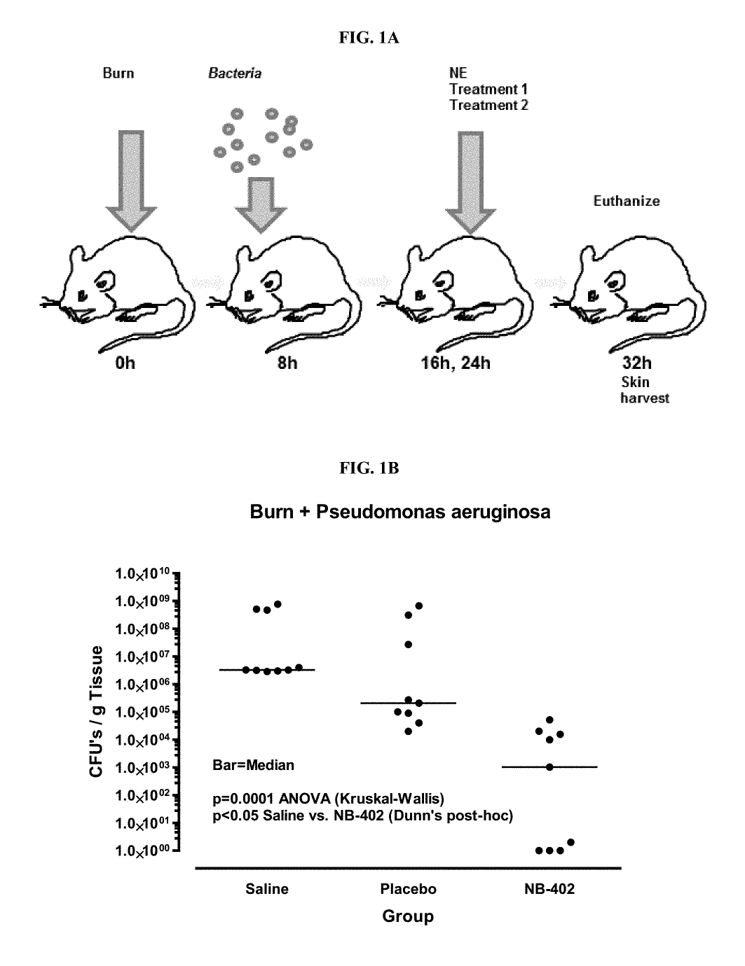

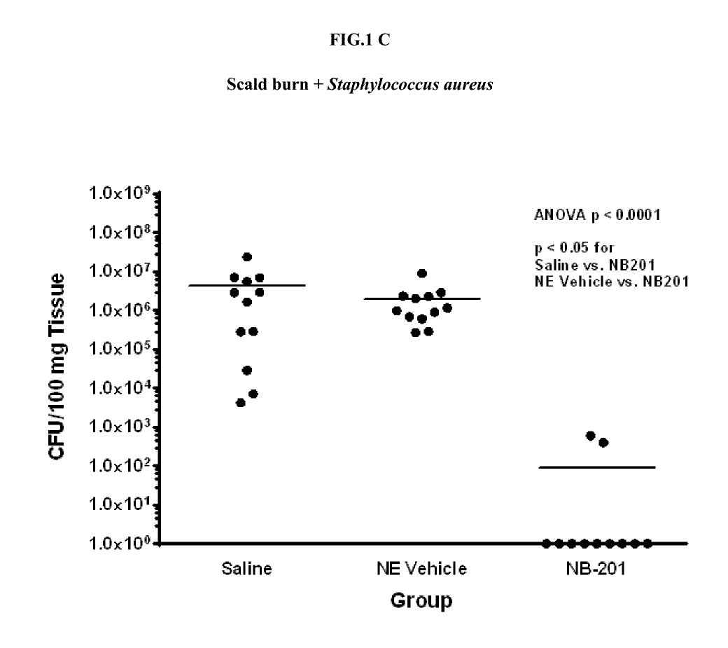

As shown in FIG. 1, topical application of NB-402 inhibited Pseudomonas aeruginosa growth in burn wounds. Spray application of nanoemulsions resulted in profound suppression of bacteria load. There was minimal pathogen growth in all NB-201 treated animals. A majority of the control (9/9) and vehicle (7/9) animals with burn injury had evidence of wound infection based on a positive quantitative wound culture with significantly more bacteria present in the wound than those animals treated with NB-402. In the clinical setting, a quantitative culture is considered to be positive when growth of more than 1×10⁵ of organisms per gram of wound tissue is documented.

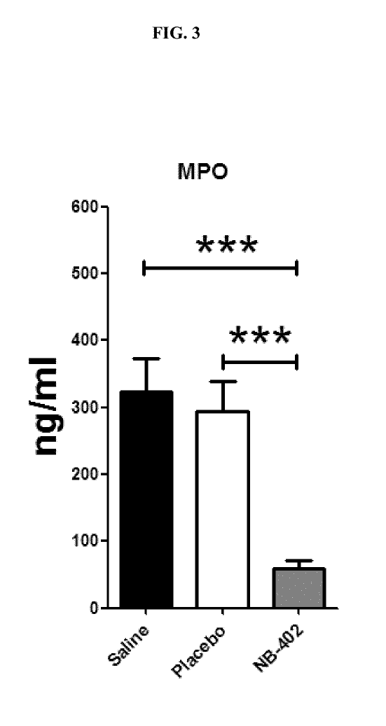

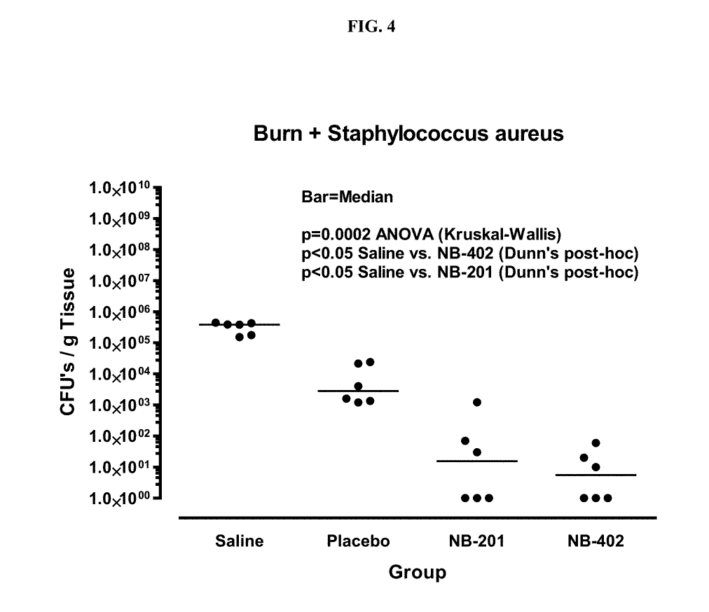

NB-402 treatment after partial thickness burn injury and Pseudomonas aeruginosa infection decreased production of dermal proinflammatory cytokines (See FIG. 2). Groups were burn+bacteria+saline, burn+bacteria+NB-402 placebo or burn+bacteria+NB-402. * P<0.05, the one-way ANOVA with Tukey's multiple comparison test. NB-402 treatment after partial thickness burn injury and Pseudomonas aeruginosa infection decreased dermal neutrophils sequestration as evidenced by myeloperoxidase assay. N=9 rats per group. * P<0.05, one-way ANOVA with Tukey's multiple comparison test (See FIG. 3).

Quantitative wound culture results for Staphylococcus aureus is shown in FIG. 4. The scatter plot represents cultured CFUs for each individual animal. The median value for each group is plotted as a horizontal line. There was minimal pathogen growth in all NB-201 or NB-402 treated rats. p<0.0002, Kruskal-Wallis test, p<0.05 for saline vs. NB-201 and saline vs. NB-402, Dunn's multiple comparison test.

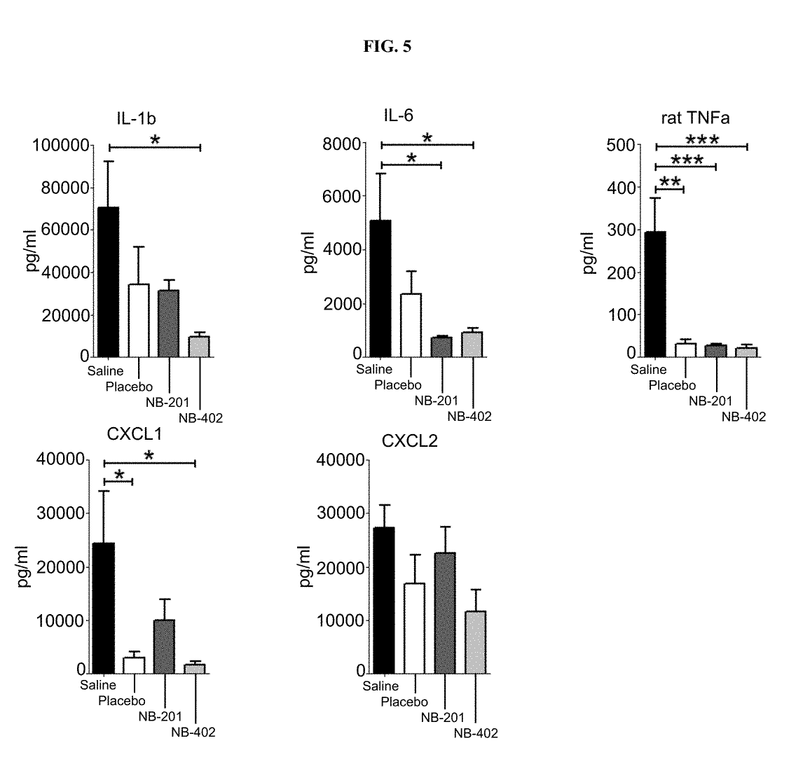

NB-201 and NB-402 treatment after partial thickness burn injury and Staphylococcus aureus infection inhibited production of dermal proinflammatory cytokines (See FIG. 5). Groups were burn+bacteria+saline, burn+bacteria+NB-402 placebo or burn+bacteria+NB-201 and burn+bacteria+NB-402. * P<0.05, one-way ANOVA with Tukey's multiple comparison test.

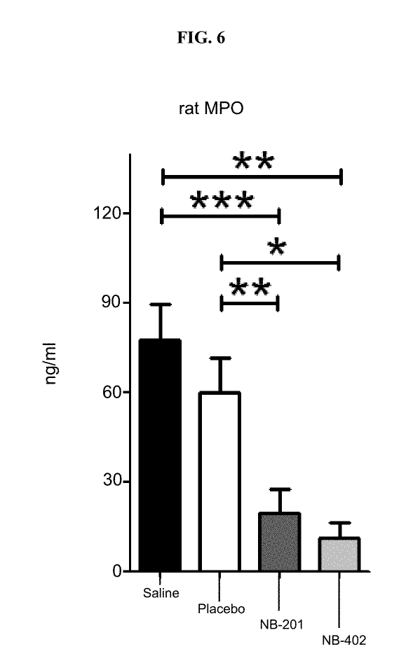

NB-201 and NB-402 treatment after partial thickness burn injury and Staphylococcus aureus infection decreased dermal neutrophil sequestration as evidenced by myeloperoxidase assay (See FIG. 6) *P<0.05, one-way ANOVA with Tukey's multiple comparison test.

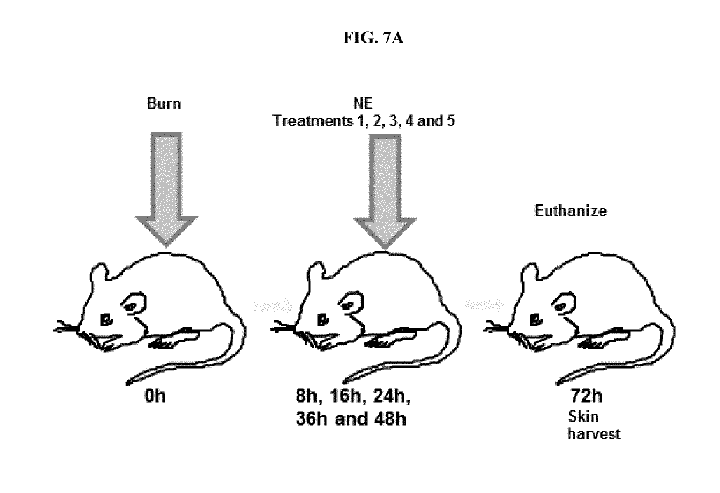

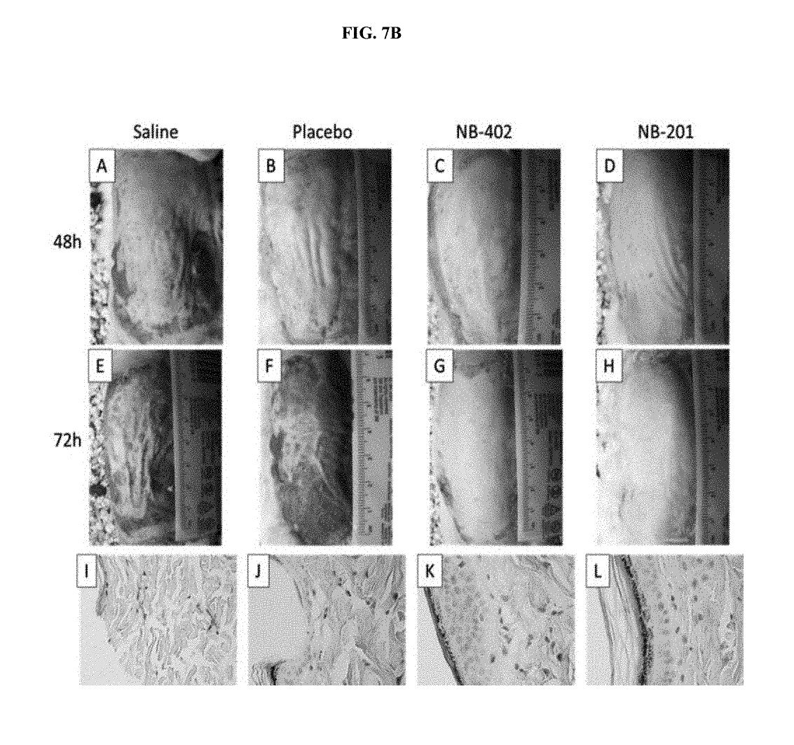

FIG. 7 shows photographic (A-H) and cross-sectional histology (I-L) analysis of burn skin, in absence of infection, after treatment with saline, placebo or NB-201 or NB-402. Photographic analysis (A-H) of Saline (A and E) and Placebo (B and F) treated rats demonstrate accentuated fibrosis and granulation tissue formation. Nanoemulsions treatment significantly reduced burn wound progression in NB-402 (C and G) and NB-201 (D and H) treated rats.

Histological analysis (FIG. 7, I-L), hematoxylin and eosin stain, original magnification ×60, revealed loss of epidermis in Saline (I) and Placebo (J) treated groups and intact epidermis in NB-402 (K) and NB-201(L) treated groups. Groups were Saline (saline treated), Placebo (NB-402 placebo treated), NB-201 (NB-201 treated) and NB-402 (NB-402 treated). Rats that received nanoemulsions (NB-201 or NB-402) experienced less discomfort compared to saline or placebo groups. Saline and placebo treated groups demonstrated significant scar formation compared to almost no scars on nanoemulsions treated rats. The NB-201 or NB-402 treated skin demonstrated no signs of fibrosis formation and appeared just like normal uninjured skin at the time of harvest at 72 hours post burn.

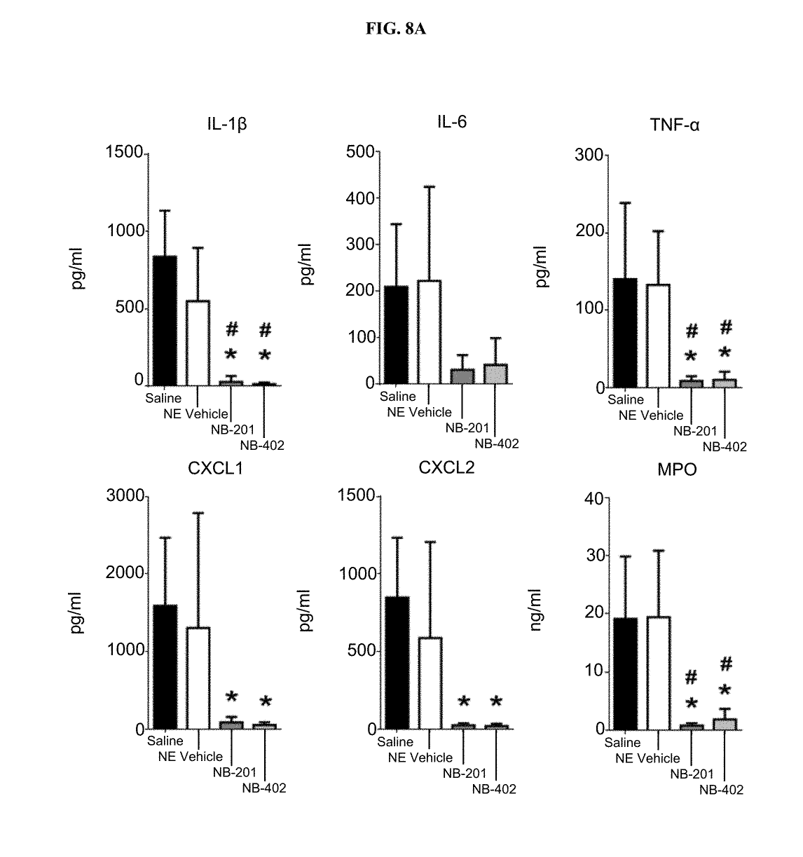

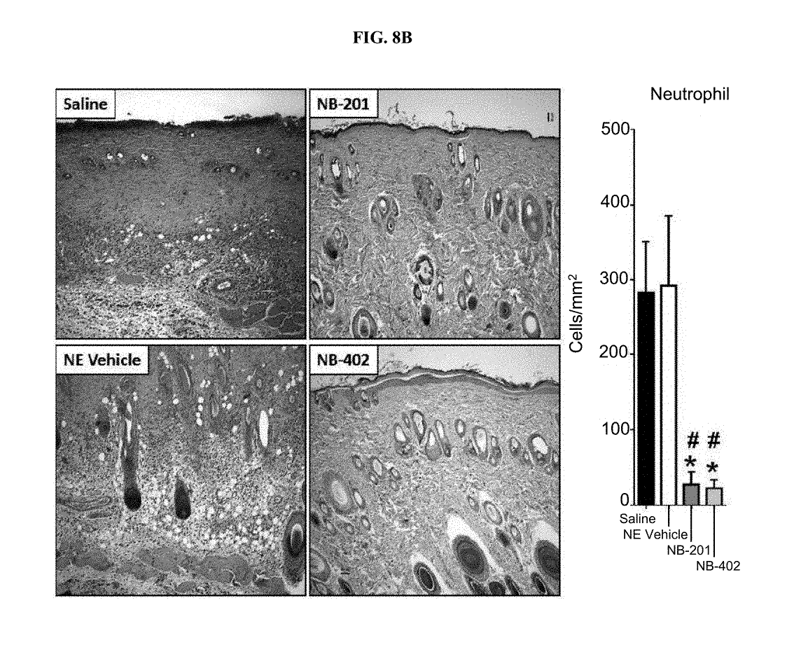

Levels of dermal cytokines measured in skin homogenates were lower in the non-infected burn wounds as compared to those in burn wounds inoculated with bacteria (FIG. 8A). Despite this, significantly decreased levels of IL-1β and TNF-α were observed in burned skin for both NB-201 and NB-402 treated animals compared to the saline and NE vehicle groups. Chemokine levels (CXCL1 and CXCL2) were reduced for NB-201 and NB-402 treated animals compared to the saline group. Once again, NB-201 and NB-402 treatment significantly diminished myeloperoxidase levels as compared to controls. Histopathologic counting of neutrophils present in skin samples demonstrated a reduced infiltration of neutrophils into the burned skin treated with NB-201 and NB-402 vs. the saline or NE vehicle treated animals (FIG. 8B). Accordingly, in some embodiments, the invention provides that treatment with NB-201 or NB-402 or other formulation described herein may be utilized to lessen dermal neutrophil recruitment and sequestration into the burn wound.

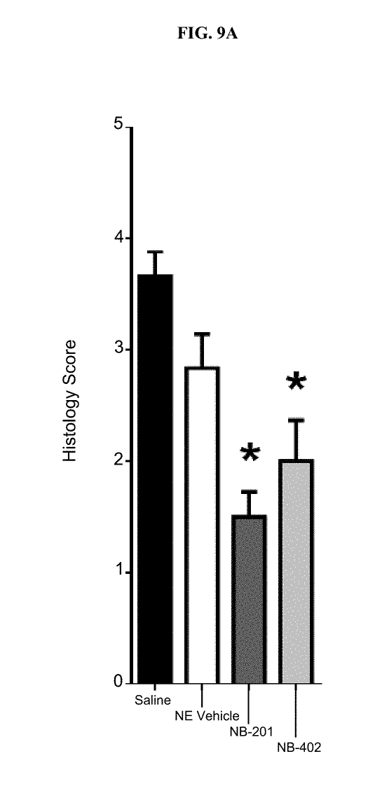

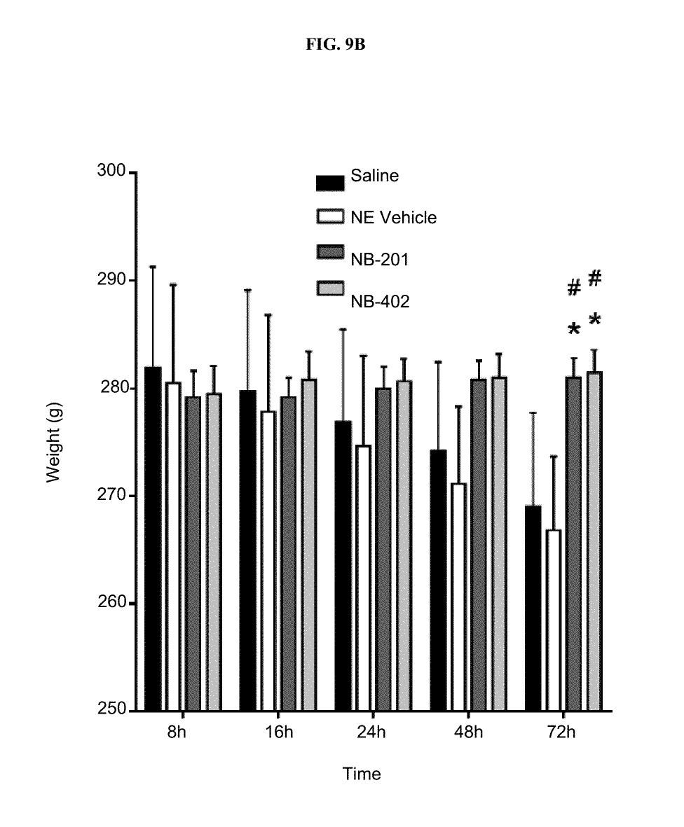

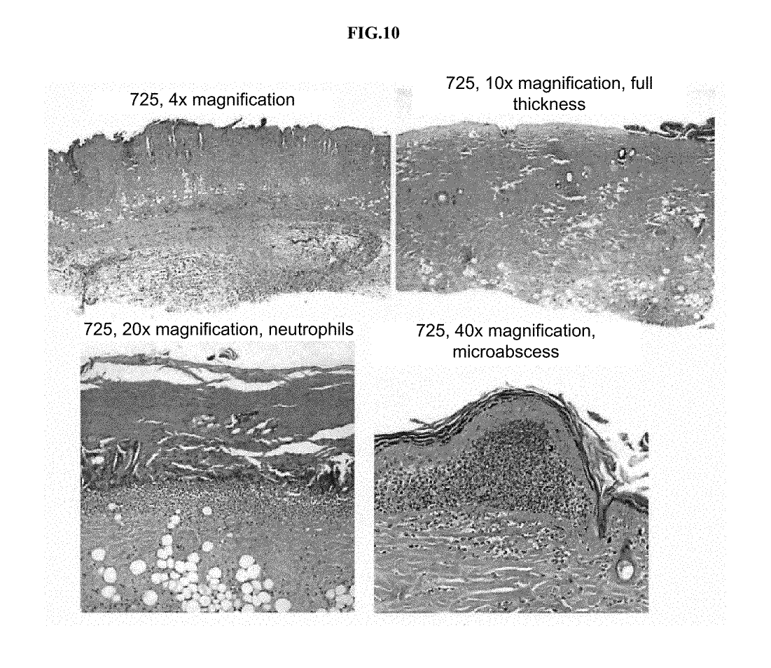

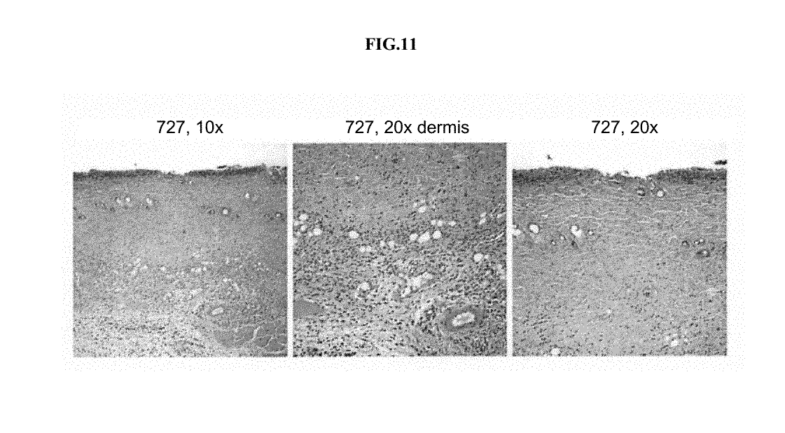

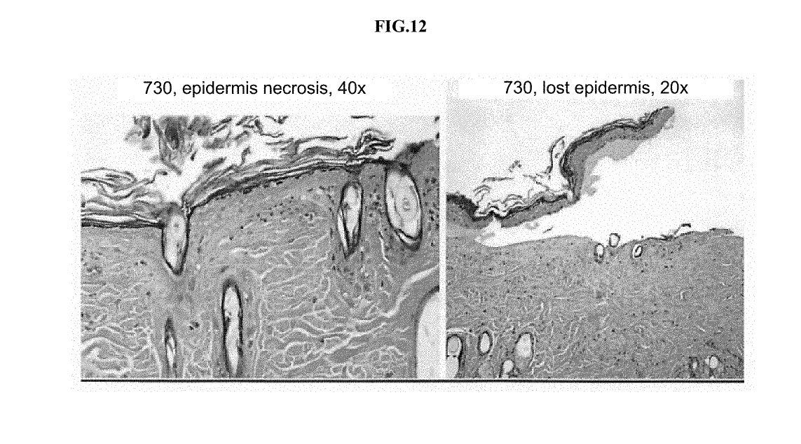

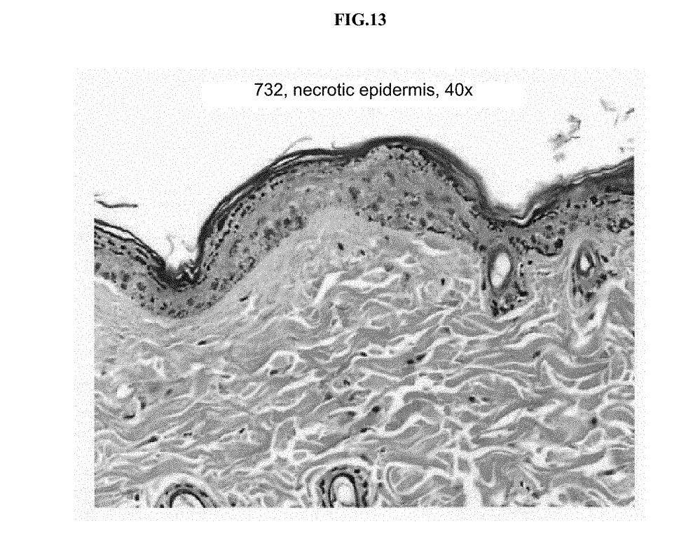

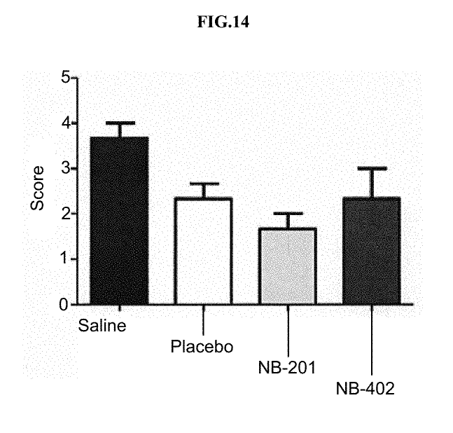

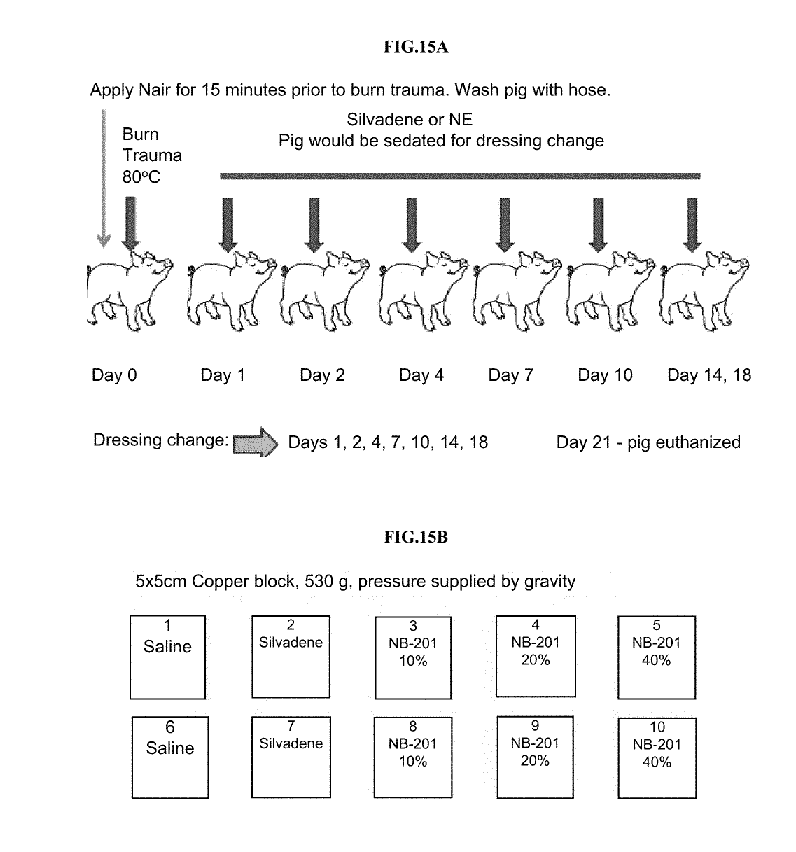

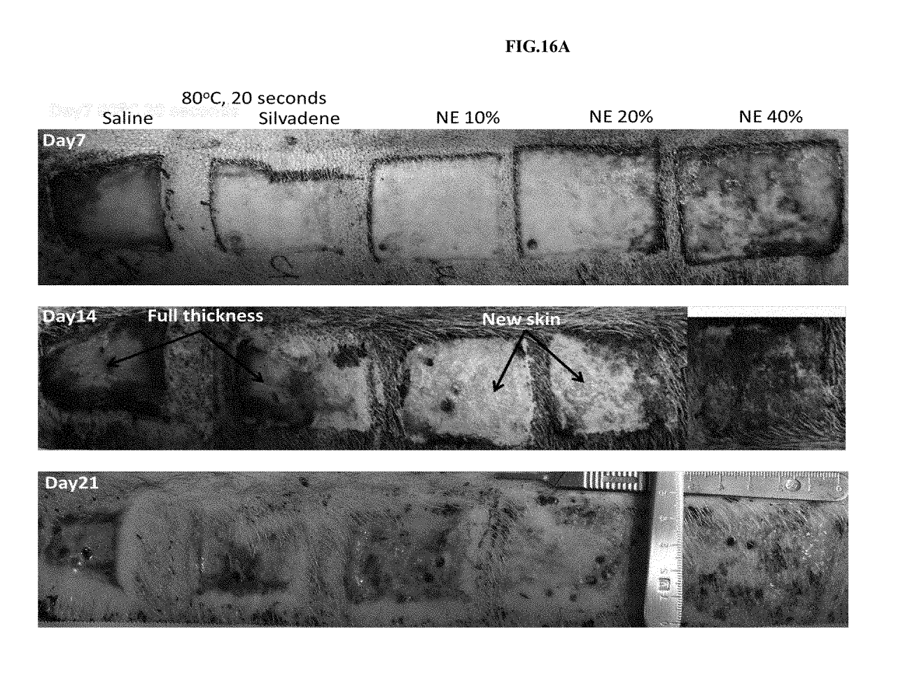

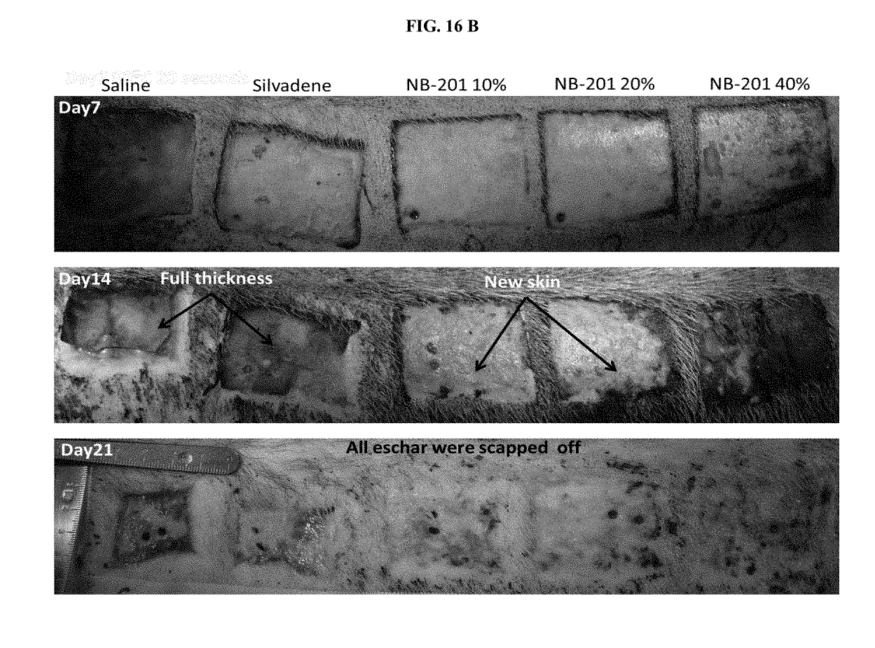

Evaluation of skin samples with a histopathology scoring system revealed significantly less burn injury at 72 hours after treatment with NB-201 or NB-402 compared to saline treated controls (See FIG. 9A). The NB-201 or NB-402 treated rats also maintained their pre-burn injury measured body weight, whereas the saline or NE vehicle treated animals lost body mass over the 72 hours of treatment (See FIG. 9B). Accordingly, in some embodiments, the invention provides nanoemulsion formulations and methods of using the same to reduce burn wound progression (e.g., of burn wound depth (e.g., as evidenced by elimination of histologic changes that occur in control and/or non-treated subjects)). Although an understanding of a mechanism is not necessary to practice the present invention, and while the present invention is not limited to any particular mechanism, in one embodiment, the invention provides that the mechanism of the anti-inflammatory effect of nanoemulsion formulations of the invention are distinct and separate from its antimicrobial activity (e.g., since anti-inflammatory effects were observed in sterile burn wounds).

### Example 5

### Porcine Burn Wound Progression and Healing Experiments

Materials and Methods.

Animal Ethics and Care. All experiments were performed in accordance with the National Institutes of Health (NIH) Guide for the Care and Use of Laboratory Animals. All animal work was reviewed and approved by the University of Michigan Committee on the Use and Care of Animals (UCUCA). Four commercially obtained Yorkshire-Landrace swine weighing between 20 and 30 kg (Michigan State University Swine Teaching and Resource Center; Lansing, Mich.) were acclimated for at least seven days before the start of the experiments. Animals were housed individually in enriched cages with water provided ad libitum. Animals were fed a standard porcine diet (Lab Diet 5801; PMI Nutrition, IN) in accordance with University of Michigan Unit for Laboratory Animal Medicine guidelines.

Anesthesia Induction. Animals were fasted overnight prior to anesthesia. Anesthesia was induced with intramuscular (IM) injection of 2.5-3 mg/kg tiletamine/zolazepam (Telazol; Zoetis Inc, Kalamazoo, Mich.) and 2.2 mg/kg xylazine and was maintained with isoflurane administered via face mask. Animals were placed in sternal recumbency for the duration of the procedure. Heart rate, respiratory rate, rectal temperature and venous oxygen saturation were monitored at regular intervals. Additional heat support was provided as necessary with a circulating water blanket.

Post-procedure analgesia was provided with injectable buprenorphine and a Butrans transdermal system (Purdue Pharma L.P., Stamford, Conn.) for systemic delivery of 5 mcg per hour buprenorphine for 7 days. A single loading dose of 0.01 mg/kg buprenorphine was administered intramuscularly immediately following burn trauma and again on days 7, 10, 14 and 18 to control post punch biopsy pain. A Butrans transdermal patch system was applied following the loading dose of injectable buprenorphine and was maintained for one week. Swine were monitored twice daily for evidence of pain. If necessary, additional analgesics were administered under veterinary supervision.

Swine Burn Model. Dorsal hair was removed using depilatory cream (Nair; Church & Dwight Inc., Princenton, N.J.) and any remaining hair was clipped. The skin was prepped with chlorhexidine scrub. A square 5×5 cm copper block (wt 530 g) with an attached positioning rod was pre-heated in an 80° C. water bath for 30 minutes prior to application to the skin. The block was applied to 10 paralumbar sites for a duration of 20 or 30 seconds per site. The block was returned to temperature in the water bath between burns. Pressure was supplied by gravity.

An occlusive dressing of sterile TELFA (Kendall Co., Mansfield, Mass.) and TEGADERM HP (3M HealthCare, St Paul, Minn.) was applied to prevent wound contamination. A clean laparotomy pad was placed over the TEGADERM to minimize adhesion of the top dressing to TEGADERM. The thorax was covered with self-adherent wrap (MEDICHOISE, Buford, Ga.) and the ends secured with heavyweight stretch tape (BSN medical, Inc., Charlotte, N.C.). A cloth jacket (MWI Veterinary Supply Inc., Rochester Hills, Mich.) was placed over the dressing to prevent fecal contamination.

Topical Burn Wound Treatment and Evaluation. Swine were anesthetized during each dressing change. The burn sites were treated with saline, SILVADENE or NB-201 (10%, 20% or 40% Table 21). Nanoemulsion formulation NB-201 was obtained from NanoBio Corporation (Ann Arbor, Mich.). 5 ml of NB-201 was applied in a uniform fashion to the burn wound surface using a spray bottle (Mistette MK 140-T; MeadWestvaco Calmar GmbH, Germany, from a 6 mL U-Save Type 1 glass vial (Neville & More, W)). For the NB-201 or saline treated groups, a 6×6 cm TELFA square was soaked with 5 ml of NB-201 or saline. For the SILVADENE treated wounds, SILVADENE cream (0.8 ml/Telfa) was applied to a 6×6 cm TELFA square. TEGADERM was then applied over TELFA squares. Dressing changes were performed on days 1, 2, 4, 7, 10, 14 and 18 after burn injury. The 4 mm full thickness skin tissue punch biopsies were performed on days 4, 7, 10, 14, 18, and 21 after burn injury. Digital pictures were taken at the time of each dressing change to monitor healing progression. Animals were euthanized at 21 days post burn.

TABLE 21
Composition of NB-201 (BAC/Tween 20)

| | | 10% NB-201 | 20% NB-201 | 40% NB-201 |
|---|---|---|---|---|
| | | Lotion | Lotion | Lotion |
| | | (BAC/Tween20) | (BAC/Tween20) | (BAC/Tween20) |
| | | 1:3 | 1:3 | 1:3 |
| Excipients | Function | (% w/w) | (% w/w) | (% w/w) |
| Purified | Aqueous | 91.921 | 81.948 | 65.790 |
| Water (USP) | Diluent | | | |
| Soybean Oil | Hydrophobic oil | 6.279 | 12.558 | 25.116 |
| (USP) | | | | |
| Edetate | Preservative | 1.894* | 1.894* | 1.894* |
| Disodium | | | | |
| Dihydrate | | | | |
| (USP) | | | | |
| Glycerol (NF) | Organic solvent | 1.008 | 2.016 | 4.032 |
| Tween 20 | Emulsifying | 0.592 | 1.184 | 2.368 |
| (NF) | agent | | | |
| Benzalkonium | Emulsifying | 0.200 | 0.400 | 0.800 |
| Chloride | agent, | | | |
| (USP) | preservative | | | |

| | | | |
|---|---|---|---|
| Total | 100.00% | 100.00% | 100.00% |

*50 mM EDTA

Nanoemulsion Formulation. The nanoemulsions (NB-201) were prepared by emulsification of a cationic surfactant, a nonionic surfactant, ethanol, a chelating agent, soybean oil, and water Benzalkonium chloride (BAC) was the cationic surfactant incorporated into the NB-201 formulation with Tween 20 as the nonionic surfactant (See Table 21). These formulations are composed of pharmaceutically approved ingredients that are included on the Food and Drug Administration (FDA) Inactive Ingredient List for Approved Drug benzalkonium chloride (BAC), the cationic surfactant incorporated in NB-201 is composed of various carbon chain lengths as following: 60% of C14, 30% of C16, 5% of C12, 5% of C18. The final concentrations of BAC in the compositions are as follows: 10% NB-201 is 0.2% BAC, 20% NB-201 is 0.4% BAC and 40% NB-201 is 0.8% BAC. The surfactants, both cationic and nonionic, reside at the interface between the oil and water phases. The hydrophobic tail of the surfactant distributes in the oil core and its polar head group resides in the water phase.

Mean particle size (Z-average) and polydispersity index (PdI) were determined for each nanoemulsion formulation. The particle size and PdI of the sample was measured by photon correlation spectroscopy using a Malvern Zetasizer Nano ZS90 (Malvern Instruments, Worcestershire, UK). All size measurements were carried out at 25° C. using disposable methyl methacrylate cells after appropriate dilution with 0.22 μm filtered deionized distilled water.

Tissue Harvest. At the selected time point after thermal injury the animals were euthanized and skin tissue samples were harvested using 4 mm full thickness punch biopsies and sterile technique. Skin samples were used immediately or frozen in liquid nitrogen.

Quantitation of Bacterial Wound Infection. A 4 mm full thickness punch biopsies were mechanically homogenized in 1 mL of sterile saline solution. This homogenate was then further diluted with 9 mL of sterile saline solution. Serial dilutions were performed, and skin homogenates were plated in triplicate on blood agar plates (Becton Dickinson). Culture plates were incubated for 24 hours at 37° C. and CFUs were counted.

Quantitation of Soluble Mediators by ELISA. 4 mm full thickness skin tissue biopsies were mechanically homogenized in 1 mL of sterile Phosphate Buffered Saline (1×), pH 7.4, containing 0.01% (w/v) Triton X (Roche) and complete protease inhibitors cocktail (Complete X, Roche, Indianapolis, Ind.). This homogenate was then centrifuged at 3000 g for 5 minutes at 4° C. and used for sandwich enzyme-linked immunosorbent assay (ELISA). Pig interleukin 1-beta (IL-1β), interleukin 6 (IL-6), interleukin 8 (IL-8), tumor necrosis factor alpha (TNF-α), and interferon gamma (IFN-γ) were measured by ELISA using DuoSets from R&D Systems Inc. (Minneapolis, Minn.). Pig myeloperoxidase ELISA kit was from TZS ELISA (Ellison Park, Mass.). Results were expressed as picograms per milliliter (pg/mL).

Histology. Fresh 4 mm full thickness skin tissue punch biopsies were fixed in 10% buffered formalin and embedded in paraffin. Sections 4 μm thick were sliced and affixed to slides, deparaffinized and stained with hematoxylin and eosin to assess morphological changes. Skin histology samples were scored by two independent and double-blinded veterinary pathologists using the following scoring system: epidermis (0=complete, 1=acute necrosis, 2=separation, 3=absent), dermal necrosis, necrotic inflammation, immature granulation tissue (0=none, 1=≤100 μm, 2=100-300 μm, 3=300-500 μm, 4=≥500 μm), perivascular inflammation (0=none, 1=mild multifocal, 2=moderate multufocal, 3=mild diffuse, 4=moderate diffuse, 5=severe), deep granulation tissue (0=none, 1=≤500 μm, 2=500-1000 μm, 3=1000-3000 μm, 4=3000-5000 μm, 5=≥5000 μm). A final score was computed by summing the scores in each category.

Statistical Methods. All statistical analysis and graph(s) were performed using GRAPHPAD Prism software, version 5.0 (GRAPHPAD Software, La Jolla, Calif.). Results are presented as mean values±the standard error of the mean (SEM) unless otherwise noted. Continuous variables were analyzed using a 1 way analysis of variance (ANOVA) and Newman-Keuls multiple comparison. Statistical significance was defined as a P value<0.05.

Results.

NB-201 limits burn wound progression in a sterile partial thickness wounds.

As described above, ten sites on the back of each pig were utilized as a scald burn site. A 5×5 cm copper block weighing 530 g was heated to 80° C. in a water bath and then applied to the burn site for 20 or 30 seconds using the weight of the block to provide consistent pressure across sites. This was repeated for each of the 10 predetermined sites on the pig. This regimen has been shown to result in full thickness injury and heals with significant scarring and wound contracture at sites treated with saline (See. Singer et al., J Burn Care Res 2011; 32:638-646).

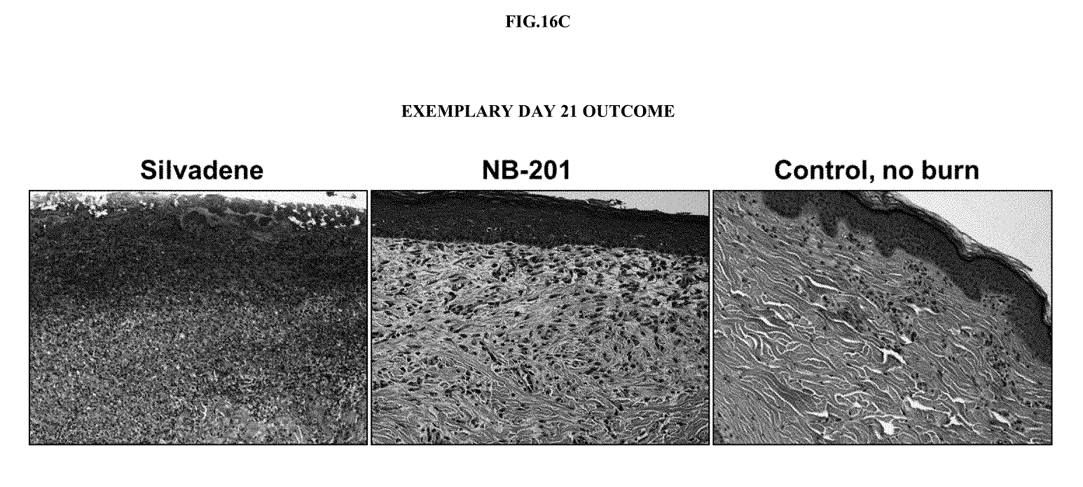

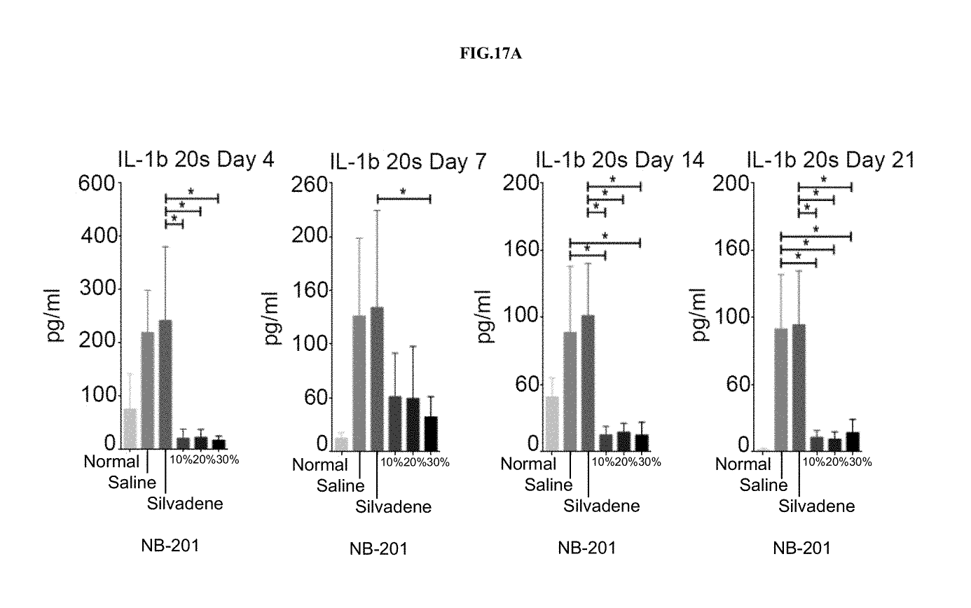

Pigs received a topical application of saline, silvadene and NB-201 immediately following partial thickness burn trauma. Dressing change were performed at days 1, 2, 4, 7, 10, 14 and 18. Digital photographs were taken to document macroscopic healing. The burn wounds treated with saline or silvadene progressed to full thickness burns by day 7, as confirmed by histopathologic evaluation, with heavy crust formation by day 14. The NB-201 treated burns had no evidence of progression toward full thickness burns (See FIGS. 16A and 16B). Macroscopic healing was achieved by day 21 post burn (see FIGS. 16A and 16B). Treatment with saline or silvadene was associated with healing by fibrosis, wound contracture and delayed healing in the wound center. In all wounds treated with NB-201 complete healing with new skin formation without scar tissue formation or skin contraction was achieved by the day 21 post burn. NB-201 treated wounds were grossly and histologically healed on the day 21 (SEE FIG. 16C). Silvadene treated wounds were not healed by day 21 and significant leukocytic infiltration was noted. Macroscopic and histopathologic appearance of wounds induced by exposure to 80° C. heated copper bar for 20 seconds or 30 seconds was not different. Both time settings resulted in very similar partial thickness wounds which progressed to full thickness wounds by day 7 unless NB-201 treated. Accordingly, in some embodiments, nanoemulsions of the invention is utilized to prevent burn wound progression (e.g., from partial thickness to full thickness wound). In other embodimens, nanoemulsion of the invention is utilized to stimulate, promote and/or generate re-epithelialization (e.g., complete re-epithelialization) of a burn wound and/or to prevent scarring resulting from a burn trauma (e.g., that is not prevented using conventional treatment (e.g., SILVADENE). While an understanding of a mechanism is not necessary to practice the present invention, and while the present invention is not limited to any particular mechanism, in some embodiments, nanoemulsion formulations of the invention significantly suppress neutrophil activity after burn injury (e.g., compared to controls (e.g., saline and SILVADENE treated wounds)) and/or significantly suppress inflammation after burn injury (e.g., compared to controls (e.g., saline and SILVADENE treated wounds)).

NB-201 suppresses production of inflammatory mediators in wounds.

IL-1 signalling is an essential mediator of postoperative incisional pain (See, e.g., Wolf et al., BrainBehav Immun 2008; 22:1072-7) and inflammatory hyperalgesia (See, e.g., Binshtok et al., J. Neurosci 2008; 28:14062-73) and can also contribute to the development of chronic pain syndrome (See, e.g., Review by Wolf et al, Pharmacol Ther 2006; 112:116-38). In-vitro stimulation of primary human keratinocytes with IL-1 resulted in production of large amounts of CXC chemokines (e.g., GRO-a and IL-8) production.

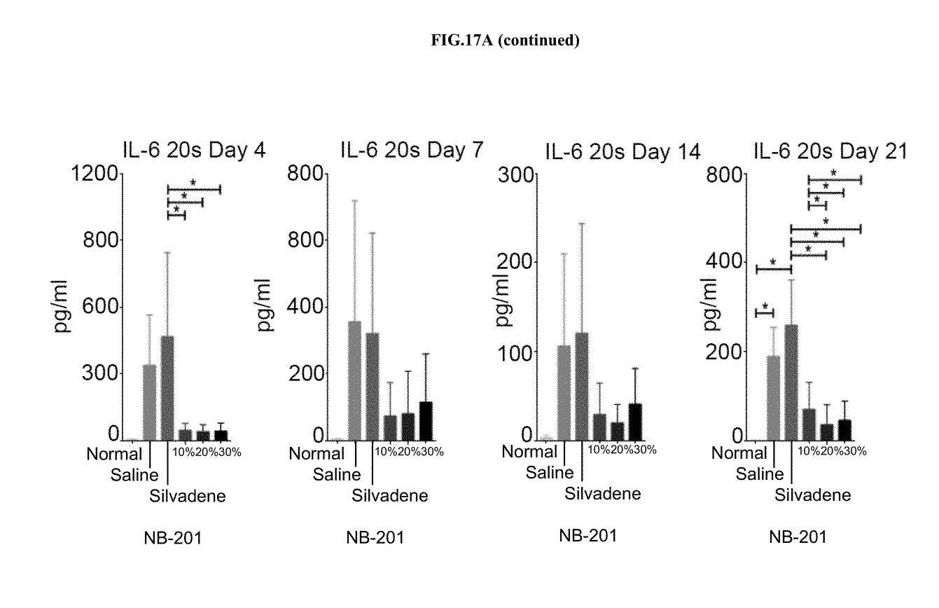

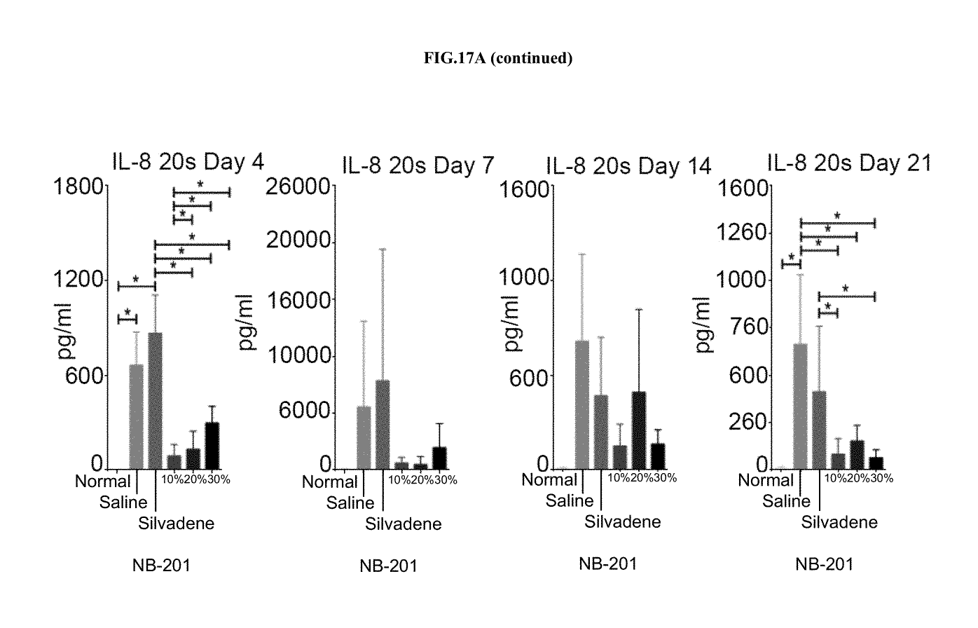

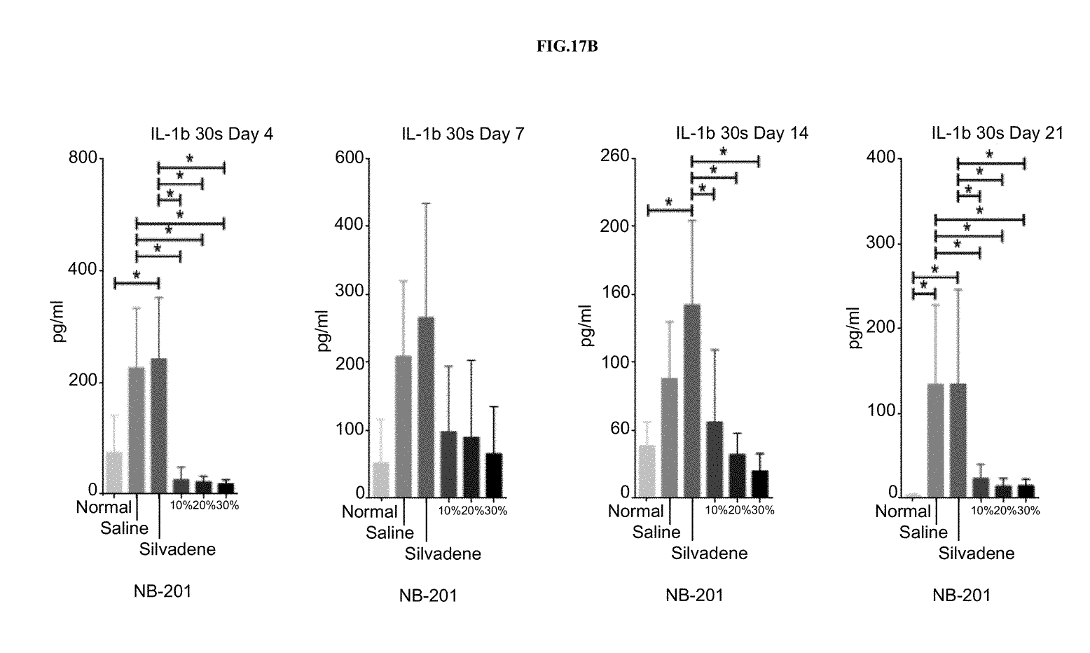

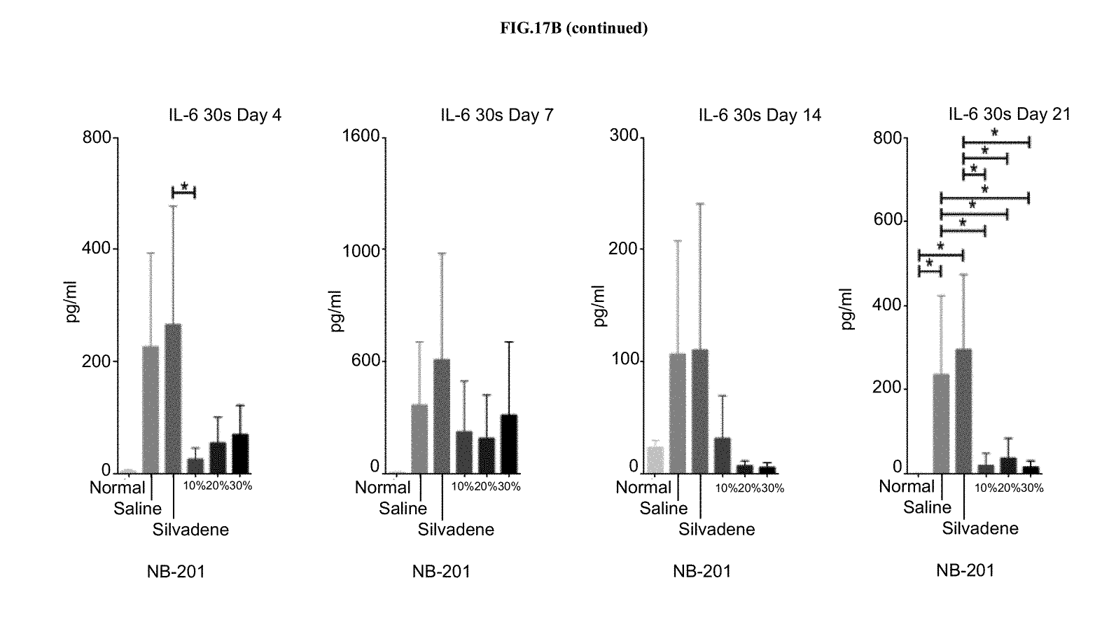

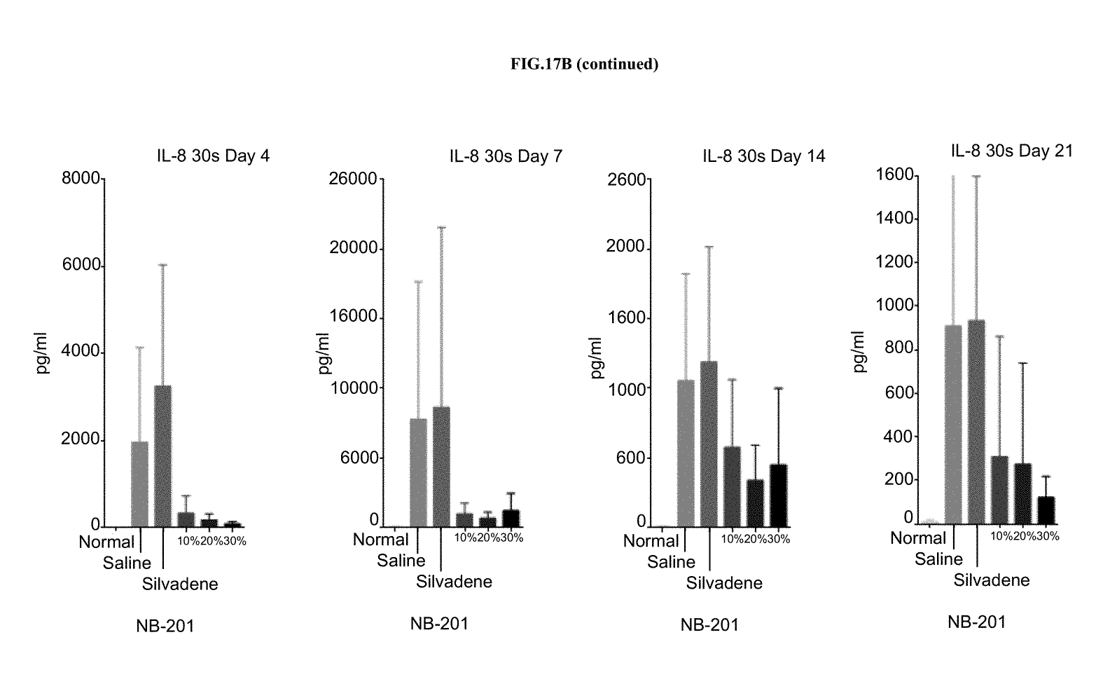

Soluble mediator production was quantified from punch biopsies obtained from wounds on days 4, 7, 10, 14, 18 and 21. In most groups, significant changes were found at days 4, 7, 14 and 21. Application of NB-201 at days 0, 1, 2 and 4 significantly reduced wound levels of IL-1β, IL-6 and IL-8 compared to silvadene treated control. The level of IL-1β compared to silvadene was suppressed on day 4 with 10%, 20% and 40% NB-201 by 12 fold, 11 fold and 14.6 fold (240.7±139.0, silvadene vs 19.7±17.3, 21.7±14.8 and 16.4±7.6, p<0.0001) in the partial thickness wounds created by 80° C. heated blocks and applied to the skin for 20 seconds (See FIG. 17A). The intense phase of NB-201 application within first week after burn trauma was followed by every 3-4 days dressing changes, leading to minor increase in IL-lb production: only silvadene vs 40% NB-201 difference was statistically significant (132.4±90.2 vs 32.4±18.8, 4.1 fold decrease, p=0.004) on day 7 (See FIG. 17A). Continued inflammation was still present in silvadene and saline treated wounds on the day 21 while NB-201 treated wounds were healed and very low production of IL-1β was found: silvadene vs 10%, 20% and 40% NB-201 was 94.6±39.9 vs 10.6±5.3, 9.4±5.5 and 14.2±9.9 correspondingly with 8.9, 10.1 and 6.7 fold suppression, p<0.0001. The changed in the levels of IL-6 and IL-8 production were similar to the trend described for the IL-1β, reaching significant difference between control(s) and NB-201 at days 4 (p=0.001, IL-6 and p<0.0001, IL-8) and 21 (p<0.0001, both IL-6 and IL-8, See FIG. 17A). Very similar trend of wound-associated soluble mediators production was found within partial thickness wounds created by 80° C. heated blocks and applied to the skin for 30 seconds (See FIG. 17B). The IL-1β production was significantly suppressed by NB-201 treatment at days 4 (p<0.0001), 14 (p=0.0003) and 21 (p<0.0001) compared to silvadene treated control (See FIG. 17B). Production of IL-6 and IL-8 was reduced as well by NB-201 application and was significant between all groups on the day 21 in case of IL-6 (p<0.0001, FIG. 17B).

NB-201 suppresses the growth of pathogenic organisms in burn wounds.

Significant bacterial contamination of both saline and silvadene treated burns was noted by days 18-21 post burn (See FIG. 18). Isolates included Staphylococcus aureus, coagulase negative Staphylococcus spp., enteric gram negative rods (not Pseudomonas spp. and non-pathogenic Corynebacterium spp. Bacteria were not cultured from NB-201 treated burns at any time point.

Histopathologic Examination.

Wound histopathology was independently examined by blinded veterinary pathologists. Parameters of epidermis, presence of dermal necrosis and necrotic inflammation, perivascular inflammation, superficial dermal inflammation and immature and deep granulation tissue were analyzed. Total histopathologic score was calculated by summing all above parameters. Biopsies were collected at days 4, 7, 10, 14, 18 and 21.

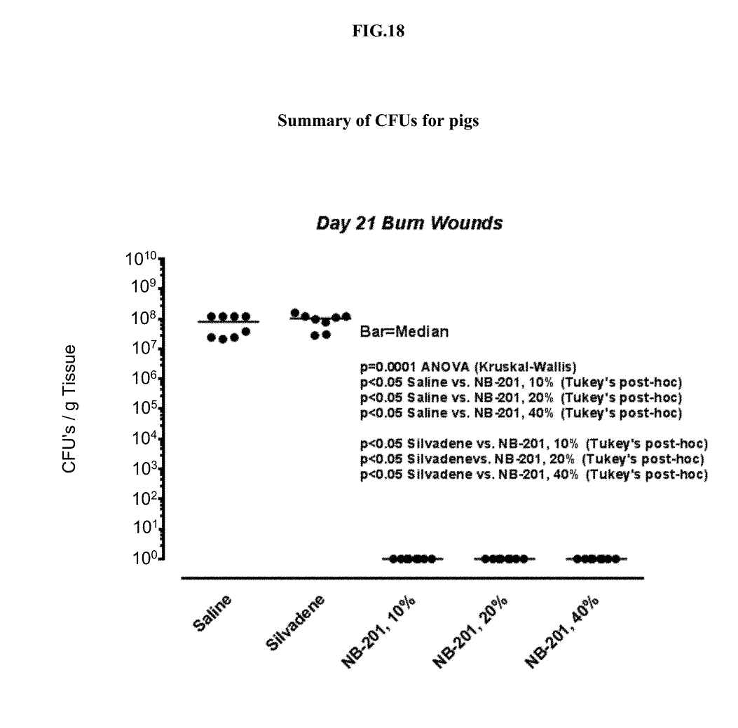

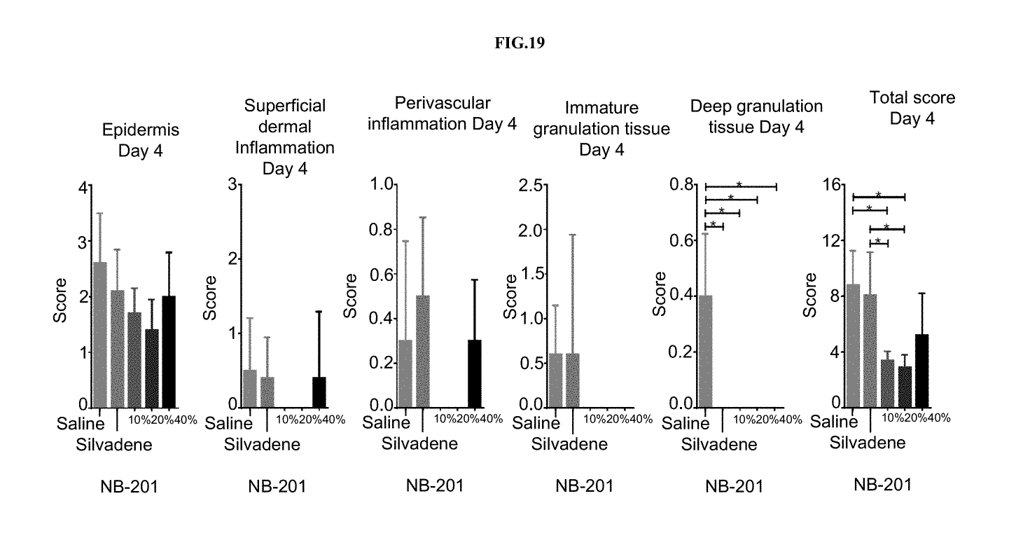

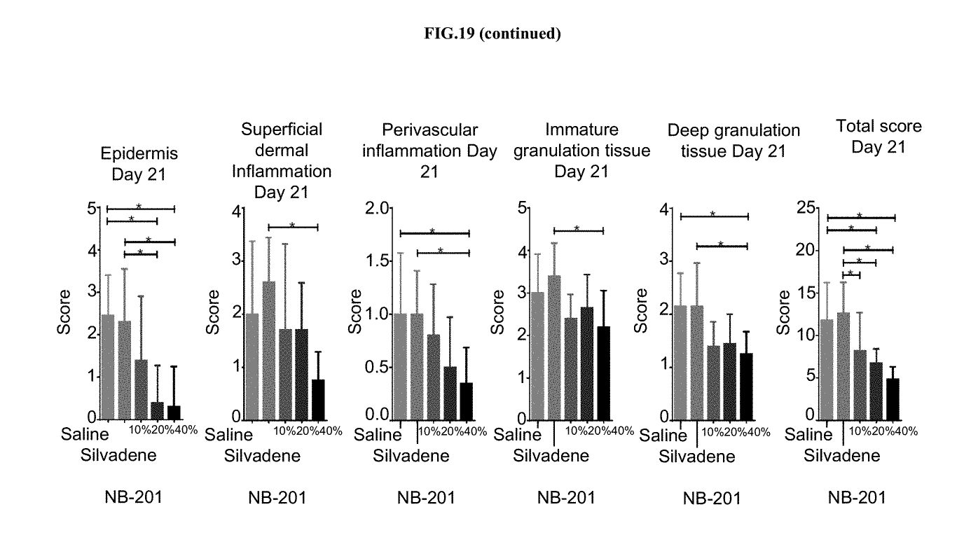

NB-201 limited damage to epidermis over the entire course of the study, reaching significance over saline treated control at day 21 (NB-201 20% and 40% vs saline, 0.4±0.8 and 0.3±0.9 vs 2.4±0.9, p<0.0001; See FIG. 19). On the day 21, superficial dermal inflammation was suppressed by 40% NB-201 compared to silvadene treated wounds (0.7±0.5 vs 2.6±0.8, p=0.01).

Necrosis was suppressed by application of 20% NB-201 compared to silvadene treated wounds on day 10 (0.5±0.7 vs 2.8±1.1, p=0.04). Dermal necrosis was evident on day 4 and no difference between control and NB-201 treated wounds was noted until day 10. At day 10 post burn, dermal necrosis was reduced by all NB-201 formulations and was not evident on day 21 (score 0) at the sites treated with 20% and 40% NB-201.

Formation of immature granulation tissue was not significantly different between groups until day 21 (Silvadene vs NB-201 40%, 3.4±0.7 vs 2.2±0.8, p=0.01). Deep granulation tissue formation was significantly reduced by 10, 20 and 40% NB-201 compared to saline control on day 4 (0±0, 0±0 and 0±0 vs 0.4±0.2, p<0.0001; See FIG. 19). On day 18, deep granulation tissue formation was significantly reduced by 20 and 40% NB-201 when compared to saline control (0.4±0.2 and 0.4±0.5 vs 3.2±1.5, p=0.005). On day 21, formation of deep granulation tissue was significantly reduced by application of 40% NB-201 compared to saline or silvadene controls (1.4±0.5 vs 2.1±0.6 or 2.1±0.8, p=0.001; See FIG. 19).

The total histopathologic scores were significantly different on 1) day 4 between saline or silvadene and 10% or 20% NB-201 (8.8±2.4 or 8.1±3 vs 3.4±0.7 or 2.9±0.9, p=0.001; See FIG. 19); 2) day 7 between saline treated control and 10% or 20% NB-201 treated wounds (12.8±1.5 vs 5±3.2 or 6.7±4, p<0.0001); 3) day 10 between silvadene and 20% NB-201 (16.9±1.5 vs 6.7±2.6, p=0.01); 4) day 18 between saline and 40% NB-201 (18.8±6.9 vs 4.7±1.9, p=0.01); 5) day 21 between saline or silvadene and 20% or 40% NB-201 (11.8±4.4 or 12.6±3.6 vs 6.7±1.6 or 4.8±1.4, p<0.0001; See FIG. 19).

NB-201 inhibits neutrophilic sequestration (MPO assay).

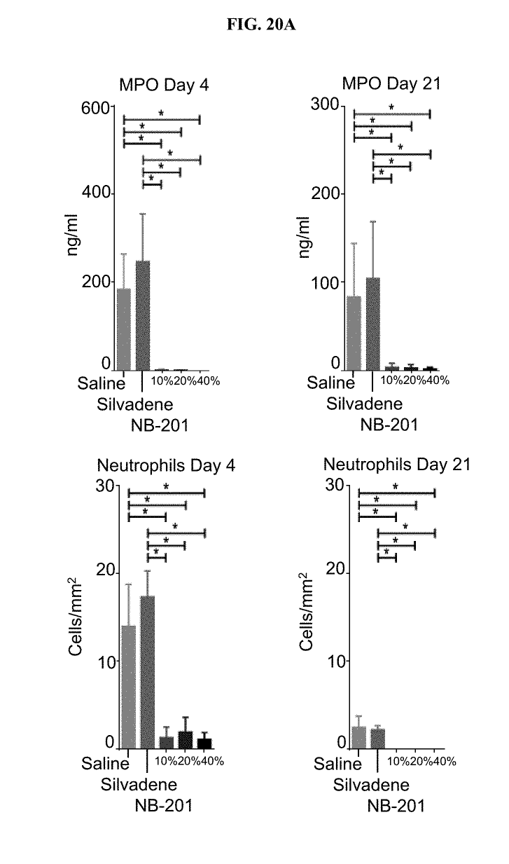

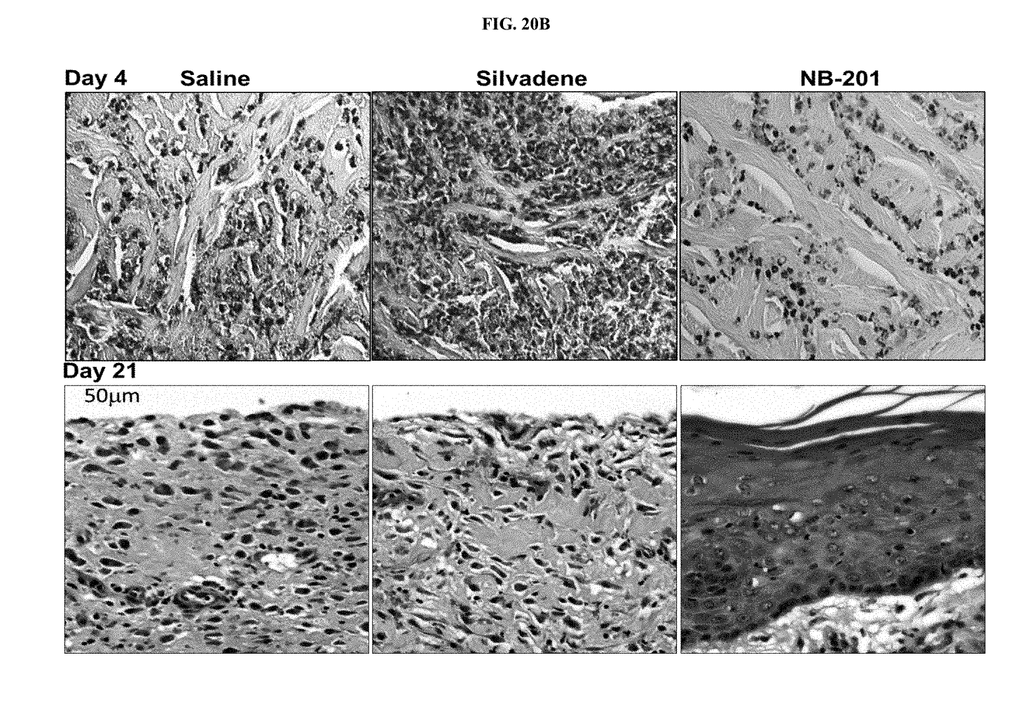

Neutrophilic sequestration associated with burn wound inflammation was significantly reduced by treatment with NB-201 compared to silvadene and saline controls on days 4 (p<0.0001) and 21 (p<0.0001, See FIG. 20A). Comparison of histopathologic scores of neutrophilic infiltration demonstrated reduced numbers of neutrophils in burned skin treated with NB-201 vs. the saline control or silvadene (See FIGS. 20A and 20B).

NB-201 preserves hair follicles and facilitates hair re-growth following burns.

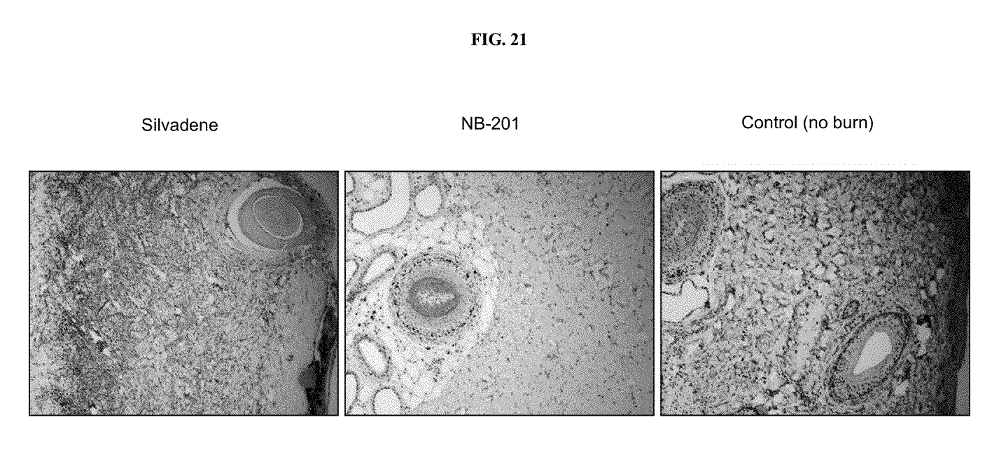

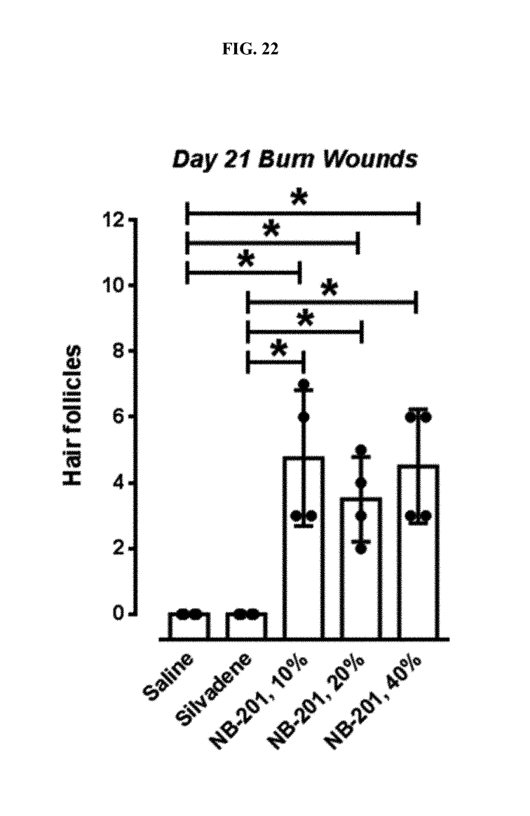

Following burn wounds, necrosis of hair follicle after silvadene treatment was observed, whereas, surprisingly, proliferation of hair follicle cells was observed with NB-201 treatment (See FIG. 21). Proliferation of hair follicle cells in burns treated with NB-201 was very similar to normal hair follicle homeostasis depicted within normal, not burned skin (See FIG. 21). The number of hair follicles per histological sample were counted at day 21 post burn. Samples from Silvadene or saline treated wounds revealed no hair follicles whereas NB-201 treated sites demonstrated 3-6 hair follicles per slide (See FIG. 22).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1.-25. (canceled)

26. A method of increasing skin regeneration within a partial thickness burn wound comprising administering a therapeutically effective amount of a nanoemulsion to the partial thickness burn wound.

27. The method of claim 26, wherein administering the nanoemulsion to the partial thickness burn wound preserves epithelial cells that line the shaft of each hair follicle within the burn wound.

28. The method of claim 27, wherein the epithelial cells within the burn wound participate in re-epithelialization of the wound.

29. The method of claim 26, wherein the nanoemulsion enhances proliferation of undamaged epithelial cells that line the shaft of each hair follicle within the burn wound.

30. The method of claim 26, wherein the nanoemulsion suppresses neutrophil sequestration and/or activity.

31. The method of claim 26, wherein the administration results in skin regeneration and wound healing within three weeks of burn wound injury.

32. The method of claim 26, wherein the nanoemulsion reduces IL-10 expression within the burn wound.

33. The method of claim 26, wherein the nanoemulsion reduces, attenuates and/or prevents bacterial growth at the burn wound site.

34. The method of claim 26, wherein the nanoemulsion reduces, attenuates and/or prevents growth of Staphylococcus aureus at the burn wound site.

35. The method of claim 26, wherein the nanoemulsion reduces, attenuates and/or prevents growth of coagulase negative Staphylococcus spp. at the burn wound site.

36. A method of treating a burn wound comprising administering a therapeutically effective amount of a composition comprising a nanoemulsion to the burn wound whereby the administration prevents ischemic necrosis and protein denaturation within the burn wound.

37. A method of preserving and/or restoring hair follicle cells within a burn wound comprising administering a composition comprising a nanoemulsion to the burn wound.

38. The method of claim 37, wherein the nanoemulsion stimulates proliferation of hair follicle cells within the burn wound.

39. The method of claim 37, wherein the nanoemulsion prevents necrosis of hair follicle cells within the burn wound.
